SEQUENCE LISTING (58 sequences)

SEQ ID NO: 1 - protein, 563 aa, Bos taurus
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 2 - protein, 563 aa, Bos taurus; identical to SEQ ID NO: 1

SEQ ID NO: 3 - protein, 563 aa, Canis familiaris
MRLRTCLLGLLALCVASKCSYSPEPDQQRTLPPGWVSLGRVDSEEELSLTFALRQQNVERLSKLVQAVSDPGSPHYGKYLTLEDVAELVRPSPLTFRTVQKWLSAAGARNCHSVTTQDFLTCWLSVRQAELLLSGAEFHRYVGGPTEIHVIRSLRPYQLPKALAPHVDFVGGLHRFPPTSSLRQRPEPQVSGTVGLHLGVTPSVIRQRYNLTAQDVGSGTTNNSQACAQFLEQYFHASDLAEFMRLFGGNFAHQASVARVVGQQGRGRAGIEASLDVEYLMSAGANISTWVYSSPGRHESQEPFLQWLLLLSNESALPHVHTVSYGDDEDSLSSAYIQRVNTEFMKAAARGLTLLFASGDSGAGCWSVSRRHQFRPSFPASSPYVTTVGGTSFQNPFRVTTEIVDYISGGGFSNVFPQPSYQEEAVVQFLSSSPHLPPSSYFNASGRAYPDVAALSDGYWVVSNSVPIPWVSGTSASTPVFGGILSLINEHRLLSGLPPLGFLNPRLYQQRGAGLFDVTRGCHESCLNEEVQGQGFCSGPGWDPVTGWGTPNFPALLKALIKP

SEQ ID NO: 4 - protein, 563 aa, Canis familiaris; identical to SEQ ID NO: 3

SEQ ID NO: 5 - protein, 563 aa, Homo sapiens; identical to SEQ ID NO: 1

SEQ ID NO: 6 - protein, 563 aa, Homo sapiens [sequence omitted]

SEQ ID NO: 7 - protein, 563 aa, Homo sapiens; identical to SEQ ID NO: 1

SEQ ID NO: 8 - protein, 320 aa, Homo sapiens
MRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 9 - protein, 556 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPTSQLC

SEQ ID NO: 10 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVNTQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 11 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHEFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 12 - protein, 563 aa, Macaca fascicularis
MGLQACLLGLFALILSGKCSYSPEPDQRRTLPPGWVSLGRADPEEELSLTFALRQQNLERLSELVQAVSDPNSPQYGKYLTLENVADLVRPSPLTLHMVQKWLLAAGAQKCHSVITQDFLTCWLSIRQAELLLPGAEFHHYVGGPTETHVVRSPRPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVTRVVGQQGRGRAGIEASLDVQYLMSAGANISTWVYSSPGRHEGQEPFLQWLMLLSNESALPHVHTVSYGDEEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVAKFLSSSPHLPPSGYFNASGRAYPDVAALSDGYWVVSNRVPIPWVSGTSASTPVFGGLLSLINEHRILSGRPPLGFLNPRLYQQHGAGLFDVTHGCHASCLDEEVEGQGFCSGPGWDPVTGWGTPNFPALLKTLLNP

SEQ ID NO: 13 - protein, 563 aa, Macaca fascicularis
MGLQACLLGLFALILSGKCSYSPEPDQRRTLPPGWVSLGRADPEEELSLTFALRQQNLERLSELVQAVSDPNSPQYGKYLTLENVADLVRPSPLTLHMVQKWLLAAGAQKCHSVITQDFLTCWLSIRQAELLLPGAEFHHYVGGPTETHVVRSPRPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVTRVVGQQGRGRAGIEASLDVQYLMSAGANISTWVYSSPGRHEGQEPFLQWLMLLSNESALPHVHTVSYGDEEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEVVAKFLSSSPHLPPSGYFNASGRAYPDVAALSDGYWVVNNRVPIPWVSGTSASTPVFGGLLSLINEHRILSGRPPLGFLNPRLYQQHGAGLFDVTHGCHASCLDEEVEGQGFCSGPGWDPVTGWGTPNFPALLKTLLNP

SEQ ID NO: 14 - protein, 572 aa, Macaca fascicularis
MTADLRKGRMGLQACLLGLFALILSGKCSYSPEPDQRRTLPPGWVSLGRADPEEELSLTFALRQQNLERLSELVQAVSDPNSPQYGKYLTLENVADLVRPSPLTLHMVQKWLLAAGAQKCHSVITQDFLTCWLSIRQAELLLPGAEFHHYVGGPTETHVVRSPRPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVTRVVGQQGRGRAGIEASLDVQYLMSAGANISTWVYSSPGRHEGQEPFLQWLMLLSNESALPHVHTVSYGDEEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVAKFLSSSPHLPPSGYFNASGRAYPDVAALSDGYWVVSNRVPIPWVSGTSASTPVFGGLLSLINEHRILSGRPPLGFLNPRLYQQHGAGLFDVTHGCHASCLDEEVEGQGFCSGPGWDPVTGWGTPNFPALLKTLLNP

SEQ ID NO: 15 - protein, 562 aa, Mus musculus
MGLQARLLGLLALVIAGKCTYNPEPDQRWMLPPGWVSLGRVDPEEELSLTFALKQRNLERLSELVQAVSDPSSPQYGKYLTLEDVAELVQPSPLTLLTVQKWLSAAGARNCDSVTTQDFLTCWLSVRQAELLLPGAEFHRYVGGPTKTHVIRSPHPYQLPQALAPHVDFVGGLHRFPPSSPRQRPEPQQVGTVSLHLGVTPSVLRQRYNLTAKDVGSGTTNNSQACAQFLEQYFHNSDLTEFMRLFGGSFTHQASVAKVVGKQGRGRAGIEASLDVEYLMSAGANISTWVYSSPGRHEAQEPFLQWLLLLSNESSLPHVHTVSYGDDEDSLSSIYIQRVNTEFMKAAARGLTLLFASGDTGAGCWSVSGRHKFRPSFPASSPYVTTVGGTSFKNPFLITDEVVDYISGGGFSNVFPRPPYQEEAVAQFLKSSSHLPPSSYFNASGRAYPDVAALSDGYWVVSNMVPIPWVSGTSASTPVFGGILSLINEHRILNGRPPLGFLNPRLYQQHGTGLFDVTHGCHESCLNEEVEGQGFCSGPGWDPVTGWGTPNFPALLKTLLNP

SEQ ID NO: 16 - protein, 538 aa, Mus musculus
MLPPGWVSLGRVDPEEELSLTFALKQRNLERLSELVQAVSDPSSPQYGKYLTLEDVAELVQPSPLTLLTVQKWLSAAGARNCDSVTTQDFLTCWLSVRQAELLLPGAEFHRYVGGPTKTHVIRSPHPYQLPQALAPHVDFVGGLHRFPPSSPRQRPEPQQVGTVSLHLGVTPSVLRQRYNLTAKDVGSGTTNNSQACAQFLEQYFHNSDLTEFMRLFGGSFTHQASVAKVVGKQGRGRAGIEASLDVEYLMSAGANISTWVYSSPGRHEAQEPFLQWLLLLSNESSLPHVHTVSYGDDEDSLSSIYIQRVNTEFMKAAARGLTLLFASGDTGAGCWSVSGRHKFRPSFPASSPYVTTVGGTSFKNPFLITDEVVDYISGGGFSNVFPRPPYQEEAVAQFLKSSSHLPPSSYFNASGRAYPDVAALSDGYWVVSNMVPIPWVSGTSASTPVFGGILSLINEHRILNGRPPLGFLNPRLYQQHGTGLFDVTHGCHESCLNEEVEGQGFCSGPGWDPVTGWGTPNFPALLKTLLNLDPFVP

SEQ ID NO: 17 - protein, 562 aa, Mus musculus; identical to SEQ ID NO: 15

SEQ ID NO: 18 - protein, 562 aa, Mus musculus; identical to SEQ ID NO: 15

SEQ ID NO: 19 - protein, 562 aa, Mus musculus; identical to SEQ ID NO: 15

SEQ ID NO: 20 - protein, 562 aa, Mus musculus; identical to SEQ ID NO: 15

SEQ ID NO: 21 - protein, 562 aa, Mus musculus; identical to SEQ ID NO: 15

SEQ ID NO: 22 - protein, 562 aa, Mus musculus; identical to SEQ ID NO: 15

SEQ ID NO: 23 - protein, 563 aa, Pan troglodytes
MGLQACLLGLFALILSGKCSYSPEPDQRRTLPPGWVSLGRADPEEELSLTFALRQQNVERLSELVQAVSDPSSPQYGKYLTLENVADLVRPSPLTLRTVQKWLLAAGARKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 24 - protein, 563 aa, Pan troglodytes; identical to SEQ ID NO: 23

SEQ ID NO: 25 - protein, 564 aa, Pongo pygmaeus
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQHPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDEEDSLSSAYIQRVNTELMKAAARGLTLLFASGDSGAGCWSVSGRHQFRPTFPASSPYVTTVGGTSFLEPFLTTNEIVDYISGGGFSNVFPRPSYQEEAVTKFLSSSPHLPPSSYFNASGRAYPDVAALSDGYWVVSNRVPIPWVSGTSASTPVFGGGILSLINEHRILSGRPPLGFLNPRLYQQHGAGLFDVTHGCHESCLDEEVEGQGFCSGPGWDPVTGWGTPNFPALPKTLLNP

SEQ ID NO: 26 - protein, 564 aa, Pongo pygmaeus; identical to SEQ ID NO: 25

SEQ ID NO: 27 - protein, 563 aa, Pongo pygmaeus
MGLQARFLGLLALVIAGKCTHSPEPDQRWMLPPGWVSLGRVDPEEELSLTFALKQQNLDRLSELVQAVSDPSSPRYGKYLTLEDVAELVQPSPLTLRTVQKWLLAAGARDCHSVTTQDFLTCWLSVRQAELLLPGAEFHRYVGGPAKTHIIRSPHPYQLPQALAPHVDLVAGLHRFPPLSSPRQRPEPQGVGPVGLHLGVTPSVLRQRYNLTARDVGSGTTNNSQACAQFLEQYFHNSDLTEFMRLFGSSFAHQASVARVVGKQGRGRAGIEASLDVEYLMSAGANISTWVYSSPGRHEAQEPFLQWLLLLSNESSLPHVHTVSYGDDEDSLSSVYIQRVNTEFMKAAARGLTLLFASGDTGAGCWSVSGRHKFRPSFPASSPYVTTVGGTSFKNPFLVTNEVVDYISGGGFSNVFPQPSYQEEAVAQFLKSSSHLPPSSYFNASGRAYPDVAALSDGYWVVSNMVPIPWVSGTSASTPVFGGILSLINEHRLLNGRPPLGFLNPRLYQQHGAGLFDVTHGCHESCLNEEVEGQGFCSGPGWDPVTGWGTPNFPALLKTLLNP

SEQ ID NO: 28 - protein, 563 aa, Rattus norvegicus; identical to SEQ ID NO: 27

SEQ ID NO: 29 - protein, 563 aa, Macaca fascicularis
MGLQGCLLGLFALILSGKCSYSPEPDQRRTLPPGWVSLGRADPEEELSLTFALRQQNLERLSELVQAVSDPNSPQYGKYLTLENVADLVRPSPLTLHMVQKWLLAAGAQKCHSVITQDFLTCWLSIRQAELLLPGAEFHHYVGGPTETHVVRSPRPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVTRVVGQQGRGRAGIEASLDVQYLMSAGANISTWVYSSPGRHEGQEPFLQWLMLLSNESALPHVHTVSYGDEEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVAKFLSSSPHLPPSGYFNASGRAYPDVAALSDGYWVVSNRVPIPWVSGTSASTPVFGGLLSLINEHRILSGRPPLGFLNPRLYQQHGAGLFDVTHGCHASCLDEEVEGQGFCSGPGWDPVTGWGTPNFPALLKTLLNP

SEQ ID NO: 30 - protein, 563 aa, Macaca fascicularis; identical to SEQ ID NO: 29

SEQ ID NO: 31 - protein, 514 aa, Saimiri boliviensis
  misc_feature (188)..(191): Xaa can be any naturally occurring amino acid
LILSGKCSYSPEPDQQRTLPPGWVSLGRADPEEELSLTFALRQQNLERLSELVQAVSDPNSPQYGKYLTLDHVADLVRPSSLTLHTVQKWLLAAGARNCHSVITQDFLTCWLSIRQAELLLPGAEFHHYVGGPAETHVVRSPNPYQLPQALAPHVDFVGGLHRFPPTSFLRQRPEPQMAGTVGMHLGXXXXVIRKRYNLTAQXXGSGTSNNSQACAQFLEQYFRDSDLAEFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAATRGLTLLFASGDSGAGCWSVSGRHQFRPSFPASSPYVTTVGGTSFQNPFRITSEIVDYISGGGFSNVFPRPSYQEEAVAKFLNSGPHLPPSSYFNASGRAYPDVAALSDGYWVVSNSVPIPWVSGTSASTPVFGGLLSLINEHRILSGRPPLGFLNPRLYQQHGAGLFDVTHGCHESC

SEQ ID NO: 32 - protein, 514 aa, Saimiri boliviensis; identical to SEQ ID NO: 31
  misc_feature (188)..(191): Xaa can be any naturally occurring amino acid

SEQ ID NO: 33 - protein, 320 aa, Mus musculus
MRLFGGSFTHQASVAKVVGKQGRGRAGIEASLDVEYLMSAGANISTWVYSSPGRHEAQEPFLQWLLLLSNESSLPHVHTVSYGDDEDSLSSIYIQRVNTEFMKAAARGLTLLFASGDTGAGCWSVSGRHKFRPSFPASSPYVTTVGGTSFKNPFLITDEVVDYISGGGFSNVFPRPPYQEEAVAQFLKSSSHLPPSSYFNASGRAYPDVAALSDGYWVVSNMVPIPWVSGTSASTPVFGGILSLINEHRILNGRPPLGFLNPRLYQQHGTGLFDVTHGCHESCLNEEVEGQGFCSGPGWDPVTGWGTPNFPALLKTLLNP

SEQ ID NO: 34 - protein, 320 aa, Mus musculus; identical to SEQ ID NO: 33

SEQ ID NO: 35 - DNA, 1689 bp, Bos taurus
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgctgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccc

SEQ ID NO: 36 - DNA, 5483 bp, Canis familiaris
cgcgtggtgtggcggtggaacatagggttcatgtgatctgtcacatgacagtggatctccagaaaggcaaaatgagactccgaacctggtgagaaatgagggggctccgggaggaggggattatgtttgttggagggtggggatacactggcagtccggctcccgctgaaacccgcctgctgcctctgcagcctcctagggctccttgctctctgtgtcgccagcaaatgcagttacagcccggagccagaccagcagcggacgtgagttgactcagcacagactgcctgccccctcccctgggccctgtcctgtgcccactgccttggctccagcctccttcaccccattaccagctcctggtaccccctccgtagtcttattcccaggttcccaggctcagtctccaagcattcacctcctgtgagtcatatctgcctctgggatcctaccccaggtctcagcccctgatcctgaaccttccatgactgatacttcccatcccccccactaacaaaggccatgttcctgacctctgaccctgcaggctgcccccaggctgggtgtccctgggccgtgtagattctgaggaagagctgagtctcacctttgccctgagacagcagaacgtggaaagattgtccaagctggtacaggctgtgtcggatcctggctctcctcattatggtgcctatcgggagaaggactggggctgggatgtgggatgcagtggagggactcagggctgggctgagtgtgggatggtgcacataatgtcagggtcatgctcttcgtgcctggggaggcggtgatatagggaagttgaatagcagtggttagtggctctcgttgaagtttctgaatttaaaaaaaagtgaactagaaataaactgtccaaaattcactcaaattcttttccccaaaacttttcacctgtgtttatcaggctagtaacctgaaagttaggagcccaggctgtgtgatcaaatctgaattcaaattctgactctactccttatcagctaccagacagatggagaagtggtagtgtcgagtacagacaggtgctcatcagctcaattccttcgccaccctcagtccaacccacgcccatgtcctcctccagtcagactctagggctgtcatttccagttctgaagctctgaaagagcctttggagtcatccactctttaccattgcttgttaccgccaactcatttgtttattcttgcatccagactgctgcccaggcccctcaggggtccgtagtctcatctgctccacctcactccccaccctgctccctactctactccaaaacagtgttctgctccagtggtcatgctggacagtgtccctccctggcacgaagcccttcatggttggggggggggggagtctcagattcaccccggagctgtgtgcatggcactgtgcaggaaagtagtgactcactgtcatgtttaggttaaaatcaacaagtatgaaaactggaactgaattagatgtcatcttataattggttctgaaagttttgaaaacttttactggaaataaacttaatatggaaaattttaatttcttaacttcatgtgttgtttatgatcatgaaaatacacgctttgatgcagtatttgcaatgttctggcctttaaggtaaatcggtatttctggccatggcattccaagtctcctcagccttttctttccctggcccccaaatgtagcttgcacctgcatagcctgcctttttgctagaaagttcccacatatcccttgagaccctgttccaggttctctgggaagtcccaatgtctcctggttgaggtccaaaatgggcaacctggaaaagcaagatcctctactgaatccctgcaggaaaatacctgaccctagaggatgtggctgaactggtccggccatcaccactgaccttccgcacagtccaaaaatggctctcagcagctggagcccggaactgccactcggtgaccacacaagactttctgacttgctggctgagtgtccggtgagagggagtgattgccccatggagggtaccaatcatcccatcagggagacaagtcactccaatgggagatggctgatagctggcgggagcttgtgggtgagagctaacacacggggagacgaggatgagcagtgatgctcaggtggcctcagctctggtctgtactgctctctgacctctgttctctgatctctgacctccagacaggcagaactgctcctctctggggctgagtttcatcgctatgtggggggacctacagagatccatgttataaggtccctacgtccataccagctcccgaaggccttggcccctcatgtggactttggtaacaaccaatggggttggtgagggttgaagcagacagagtcaggggctggaaaaatttaggtgccgtggggggcttggggtgggatcaatggtaaagtgggaattggagtctaaaagatctcctcctcaagcctgacctctccccacagtgggcgggctgcaccgcttcccccccacatcatccctgaggcaacgccctgagccacaagtgtcagggactgttggcctgcacctgggggtgaccccatctgtgatccgtcagcgatacaacttgacagcacaagatgtgggctctggcacaaccaacaacagccaagcctgtgcccaggtgagccaggcagagaattccagggtcctaacaccctacatgtctccccaggttgggcttgctctctgactcctcccctgggttctcaccctctaactggaacaacatcccctgccctgactttggggctgcatcctctgacccatgatatctgctctgattccttttgtagttcctggagcagtatttccatgcatcagacctggctgaattcatgcgcctctttggtgggaactttgcacaccaggcatcggtagcccgtgtagttggacagcagggccggggccgggctgggatcgaggccagtctagatgtggagtacctgatgagtgccggtgccaacatctccacctgggtctacagtagccctggtactgccaagggggactggtgggtggggaaaggggtggtggtgagtggtgatcactgctcccgcaagggaatcccatgagctagacagagatcctgacaacgtccttgtgttccttcccttcccctaaatccaggccggcatgagtcacaggagcccttcctgcagtggctcctgctgctcagtaatgagtcagccctgccacatgtgcacactgtgagctatggagatgacgaggactccctcagcagcgcctacatccagcgggtcaacactgagttcatgaaggcagccgctcggggtctgaccctgctctttgcctcaggtgacctcccactctaaacttagacccttctggaatccaggcattttgaccccacagagacccttgtgatctctgacttataatctgaactcaccagggacccggatctgacctctaaactctgagctcttgcagcaatgactgtttaatttcttctctgacatccgaacccacatctcaagctctgacccattgatctgaatgctaaacttgctctttctctcaggtgacagtggggctgggtgttggtctgtctctagaagacaccagttccgtcccagcttccctgcctccaggtaagcactccagcccacctcttacctgtgaccgccaatctttcatctatgacctcatgatctggaacctttgccttgaccccaccaggtgctcctgtagcttaagacctcagtttaactggctctggttgctgcattcccctcttcttcctataaatctaggtcccatggcctctaagtagaacaaagttcccagtattcctcttccttagttcccaggcatcagagtagaagatttggtgaatgttctatagagaaaagtatgcactgtcacctcaggcccacgccttgagggcttcgtcttccccatgtacctgcttccccacagagtccagcccctctagcaagacctagcccatactcccccatctccacatattttggaggtccccttgagaccaccacaatctgaatgctgttttctgctctcagcccctatgtcaccacggtgggaggcacatccttccagaatccatttcgagtcacaactgagattgttgactatatcagtggtggcggcttcagcaatgtgttcccacagccttcataccaggtatgtatgtgtatttatggggatagatggtaggagggatcctcagttaagcagaccatactcactcaacaacacctttcaggaggaagctgtggtccagttcctgagctccagtccccacctaccaccatctagttatttcaatgccagtggccgtgcctatccagacgtggctgcactctccgatggctactgggtggtcagtaacagcgtgcccattccatgggtgtctggcacctcggtaagaatcagcctggctccaaaccccctctcaggaactatcctcatcctctactaagaccttgcacccagaacccctgcctcctctaagacctctgatccttttaaacatccgtccctgtccctgaggggagcctgagtggctcagcggtttagcgccaccttcggcccaggttgtgatcctagaggcctgggatcccacgtcgggctccctgcatgaagcctgcttctccctctgcctgtctctgcctctctctctctctcactctgygtctttcatgaataaataaaatcttaaaaaaaaatccatcccccaaatccaatcactttgaacccctaacctctacttacactcttactcttgcaggcttctactccagtgtttggtgggatcctatccctgataaatgaacatagactcctcagtggcctccctcctcttggctttctcaacccaaggctctaccagcagcgtggggcaggactctttgatgtgagtatggaagggaaaggaaggtgtggacattttcatgaatatggggagtgcaaacatgaacttgaggggagttctgatggggtcctgaaatatgaatacagggggctttagtctgtaagtactggtacagacaggcagccccagatctgatgcccaccttctccctaggtaacccgtggctgccatgaatcctgtctgaatgaagaggtgcagggtcaggggttctgctctggccctggctgggatcctgtgacaggctggggaacacccaacttcccagctctgctgaaggcactaatcaaaccttgaccctttcctgcctggacactgaactggtcccctgccccatagctggtggcttggtctctaattctgcactgtcggaaggcctgttgaaccctcaaccatagactgcaacagacagcttatttccctaaccctgaaatgctgtgagctgacttgactcctaaccctaccctgctccatcatgctcaggtcttcctactccg

SEQ ID NO: 37 - DNA, 3487 bp, Homo sapiens
cgcggaagggcagaatgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccattttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgctgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctgaccctttcctatcaggagagatggcttgtcccctgccctgaagctggcagttcagtcccttattctgccctgttggaagccctgctgaaccctcaactattgactgctgcagacagcttatctccctaaccctgaaatgctgtgagcttgacttgactcccaaccctaccatgctccatcatactcaggtctccctactcctgccttagattcctcaataagatgctgtaactagcattttttgaatgcctctccctccgcatctcatctttctcttttcaatcaggcttttccaaagggttgtatacagactctgtgcactatttcacttgatattcattccccaattcactgcaaggagacctctactgtcaccgtttactctttcctaccctgacatccagaaacaatggcctccagtgcatacttctcaatctttgctttatggcctttccatcatagttgcccactccctctccttacttagcttccaggtcttaacttctctgactactcttgtcttcctctctcatcaatttctgcttcttcatggaatgctgaccttcattgctccatttgtagatttttgctcttctcagtttactcattgtcccctggaacaaatcactgacatctacaaccattaccatctcactaaataagactttctatccaataatgattgatacctcaaatgtaagatgcgtgatactcaacatttcatcgtccaccttcccaaccccaaacaattccatctcgtttcttcttggtaaatgatgctatgctttttccaaccaagccagaaacctgtgtcatcttttcaccccaccttcaatcaacaagtcctcaatcaacaagtcctactgactgcacatcttaaatatatctttatcagtccacaagtccttccaattatatttcccaagtatatctagaacttatccacttatatccccactgctactaccttagtttagggctatattctcttgaaaaaaagtgtccttacttcctgccaatccccaagtcatcttccagagtaaaatgcaaatcccatcaggccacttggatgaaaacccttcaaggattactggatagaattcaggctttcccctccagcccccaatcatagctcacaaaccttccttgctatttgttcttaagtaaaaaatcatttttcctcctccctccccaaaccccaaggaactctcactcttgctcaagctgttccgtccccttaccacccctgatacaactgccaggttaatttccagaattcttgcaagactcagttcagaagtcaccttctttcgtgaatgttttgattccctgaggctactttattttggtatggctgaaaaatcctagattttctaaacaaaacctgtttgaatcttggttctgatatggactaggagagagactgggtcaagtaagcttatctccctgaggctgtttcctcgtctgttaagtgtgaatatcaatacctgcctttcataatcaccagggaataaagtggaataatgttgataacagtgcttggcacctggaagtaggtggcagatgttaacgcccttcctcccttgcactgcgccccctgtgcctacctctagcattgtaacgaccacatagtattgaaatggccagtttacttgtctgccttcctttccaagaccgttggtgcctagaggactagaatcgtgtcctatttaactttgtgttcccaggtcctagctcaggagttggcaaataagaattaaatgtctgctacaccgaaacaaa

SEQ ID NO: 38 - DNA, 7947 bp, Homo sapiens
ggatccacgcttagtgggaaactggagggatcaatggacagagagaagtcagattaataggagtgaggaaaaacaatggcttgaaaaaacagaaggttatagtctaagaaagcattctataattataatttcaaaggtagagcagttttgagagatagcaagcccaatgcagccatgggactggtttgcttaagtgaagtgaaggtaatgctcactgggtttaatgagatgaagaaatggaaggttagggtgttgaatgggtcttttacctagatattttgatcatctaggtcaaatgatcgcaggagttgggatggacagaaaggtgaagtgccataatcttcagtgtgggaatataatctataggttaatggaagccagtggcctgtaggacaacagcaggagtagagggatctatctaaaaagtattgaggtcagaacaagggatttcacatgagtattggattaatggttttggaagttaaaatgacactgctgacaccatcttccatatggatttttggggggaaaaaaaggaagactggaagggaattgaatcctcagggttttaactctcattgaaaatcctcttgtaatgaccccagttggaagtagtaaactcagacctccttgtgggctttgagttaccattagaagaagcaaaggtccgtttccattttatccacctgagcccactaaacaagatgcaagattcctccatggcccttagaacaaagttgatttccttgaaaaccttgggaacccataataaacaggaccccagctgattgtagatctcctgggaagctacttaaaactggaaactgatttgaataaaaggatcctggtcttgcaaaagctttactctgttcataaatgtagtcgtaaaatgaagacaaaattgataatatggggacataaaggataaaagaggaaaaaaattttttcactgcaatctttcgaggcagatcacttttatataagtctccatattgtaaatgcagaaacctaggctctgagaggttattttgctgatgtcatacagctaggaagaaccccagtctgtgggtctctacagcccactcacttgtggtgattcccaggggaccaggctcaggacgcaggtggggagccctgggcttactcagtgcttgactggccagaggggagaatccgggtggcggccccacccttgcctagcatttgggaccacccatgcaagggaggagccagtaccgtcactagttactaggcagaggggtagtggtggtggaatatagagctcatgtgatccgtcacatgacagcagatccgcggaagggcagaatgggactccaagcctggtgagaaattgagagggctcgggagaaagggatcacgttggagggagcacacattgggagggtgggaacacaaggacaacgtagctcccactgaaacccacctgctgcccctacagcctcctagggctctttgccctcatcctctctggcaaatgcagttacagcccggagcccgaccagcggaggacgtgagttgacttagcacagactgcccccttccccataccctgttctgcctcccactgtcctggtcctagcttctctcacccccgtgccagctcctagtacgcactccatagcttcatctctatgttcctagcctcagtccccttcattcacctcatgtgatctgtatctgctcctaggatcctccccaaggtctcagctcctaatctggaaccttccatgaccaatattttccatctccaccctaaccaaagccatgtccctgacccctgaccctacaggctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggtgccttttgggactgaggacaggatgtgggatgcggtggagggacacagggctgggttgggcatggaatggcgatcatgtcagagcctgccaagacacttgtgttcctcaggctagaaacctaaaaggggatgtggttcagtatacaggctttatgatcaaatacgaactcaaattctgactctgctacttactagctaccaggcatctagtacaacttacgttctttcccctaccctcaaatccattctaatttcctcttttgttcacatactaaggctgccagttctaactcccaaagagcctttggattaatctccttcttgccgtcttttgttacgaccaactccgttatttattcccactcctggactactgcccagctccccagctgatctgcagtctcctccctccaccccacccctcactgtactcccccactctgcttcaaaacagtctctccaacagtcaaaatggattggtctctctgtactcctacagcaggggagtgtgtgcttgtgaactgcagaggctggggacggggcagtgtgacatagtgtgcatggcaggaaacagtgactcaccattcgtgttaagcttaaaatcaaaaagtatgcaaatttgagtaaactgaataactggtgccgaaagatttgaaaacatttaccggacataagcttaaaattaaaatggaaaatttatatgtattaactcattcattttcatgatcatggcaatacatgctgtggtgcgacatttgcaacttactggcctttagggtaaatccatattattggccatggcatttcaagtctctccagccttttcttctctgcttcccaagtacacctacacctgcacacatagccctcctttaccctgttccaggctttgggaagtcctgatgtctcatagttgaggtccaaaagggggagtttgggaaagcaatgaatgagggcaagtgcctcttctgaatccctgcaggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccggtgagaggaaatgattgctccatggagggcaccagtcatcccatcagtgagatggatgggagggagttgagagcttgctggggcttgtgggtgggagctaatgcatggggagacagtgactgactgcccagggatgctcagaggtagcttcttctgttccgttttgagctttctgacctctgttctctgacctccagacaagcagagctgctgctccctggggctgagtttcatcactatgtgggaggacctacggaaacccatgttgtaaggtccccacatccctaccagcttccacaggccttggccccccatgtggactttggtaacacctatggggtgaatgggggatggggcacacagatccaggggctgaggaagtttagatgccattggggactgggggtggggtggttgtaaggtgggcattacagtctataagatctcctcaagcctgacttctccctacagtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccaggtgagccaagcaaagagccccagggtcctcatagcctccccacagtgtcctcaattccttaccaccctgggactcaccctcggacccacgatctctgctctgactccctccatagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggtactaccaagaggactggacagtggggaagggggtgggagatgggtgttgatccctgctccctcaagggaatgctataagctggagagagatcctgacaacccccagtgactatctttgtgcccatccctcaaaaaaaaaaaaaaaaaaatccaggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacctcctaccctaaacttagacaacgcttacacctctgcacgcctgggtgctttgactccacagtgatccctgagcctggtctctgactcataatctgacactcagaccttccagtagggaccactgacctgacctctacactctgacctcctacagtaacaaatttcccctctgacatccgaacccacatactaagccctaaccaattaatatgaatgctacacttggtctctctcaggtgacagtggggccgggtgttggtctgtctctggaagacaccagttccgccctaccttccctgcctccaggtaagtactctagcctaccactcaggtataaccaccacctttcacttgtgatctcatgatgtagaacctttgtcttgaccccaccatgtgctcctgtggttcagccttaagctttgcctgccctggttgctgtactcctgtctcttcttcctgcaggtcccaggccccaaatctcttgtgtgggatacagggtcatagctgttccttttcgtcagttcccaggcatttgagtggaagatttggtgggtgttctgtacagaaaagtgtgcacagtcacctcaggccatgccttgaaggctcaaaatctcttagtcaatcccatatacatgcttccccacagagtctagttcctccagcaagacctgggctatactcacccctccccacatatcttggaggtccccttgggtcccctactatccaaatgctgtcttctcccctcagcccctatgtcaccacagtgggaggcacatccttccaggaacctttcctcatcacaaatgaaattgttgactatatcagtggtggtggcttcagcaatgtgttcccacggccttcataccaggtacgtgtgtttgtgtggatggatgcagggtaagagtgaggatgggggatcctcagttcagctgactgctgggcaggccacatgccaatactcactcaaaaatgcctttcaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggtgagaatcagcccatctccaaactctcactcaggaactacccttaccccctaacaccttgaacaccttgcacctagaacccctgactccttagagatgtctgatactttaaagcatcactcccaaaaagtccaatcactcagaacccctgaccttacttgcaccttcactcttgtaggcctctactccagtgtttggggggatcctatccttgatcaatgagcacaggatccttagtggccgcccccctcttggctttctcaacccaaggctctaccagcagcatggggcaggactctttgatgtaagtatggaagggaagggtgtggacgttttcaaacaactatggggagtgctaagggggacttgggggcagttagggtggtgtggaatagcctttgaaatgtgagtacagggtgaggagatatactctttaagtactggtactagtaggcccagatctgatgccagcctcctccctaggtaacccgtggctgccatgagtcctgtctggatgaagaggtagagggccagggtttctgctctggtcctggctgggatcctgtaacaggctggggaacacccaacttcccagctttgctgaagactctactcaacccctgaccctttcctatcaggagagatggcttgtcccctgccctgaagctggcagttcagtcccttattctgccctgttggaagccctgctgaaccctcaactattgactgctgcagacagcttatctccctaaccctgaaatgcggtgagcttgacttgactcccaaccctaccatgctccatcatactcaggtctccctactcctgccttagattcctcaataagatgctgtaactagcattttttgaatgcctctccctccgcatctcatctttctcttttcaatcaggcttttccaaagggttgtatacagactctgtgcactatttcacttgatattcattccccaattcactgcaaggagacctctactgtcaccgtttactctttcctaccctgacatccagaaacaatggcctccagtgcatacttctcaatctttgctttatggcctttccatcatagttgcccactccctctccttacttagcttccaggtcttaacttctctgactactcttgtcttcctctctcatcaatttctgcttcttcatggaatgctgaccttcattgctccatttgtagatttttgctcttctcagtttactcattgtcccctggaacaaatcactgacatctacaaccattaccatctcactaaataagactttctatccaataatgattgatacctcaaatgtaagatgcgtgatactcaacatttcatcgtccaccttcccaaccccaaacaattccatctcgtttcttcttggtaaatgatgctatgctttttccaaccaagccagaaacctgtgtcatcttttcaccccaccttcaatcaacaagtcctcaatcaacaagtcctactgactgcacatcttaaatatatctttatcagtccacaagtccttccaattatatttcccaagtatatctagaacttatccacttatatccccactgctactaccttagtttagggctatattctcttgaaaaaaagtgtccttacttcctgccaatccccaagtcatcttccagagtaaaatgcaaatcccatcaggccacttggatgaaaacccttcaaggattactggatagaattcaggctttcccctccagcccccaatcatagctcacaaaccttccttgctatttgttcttaagtaaaaaatcatttttcctcctccctccccaaaccccaaggaactctcactcttgctcaagctgttccgtccccttaccacccctgatacaactgccaggttaatttccagaattcttgcaagactcagttcagaagtcaccttctttcgtgaatgttttgattccctgaggctactttattttggtatggctgaaaaatcctagattttctaaacaaaacctgtttgaatcttggttctgatatggactaggagagagactgggtcaagtaagcttatctccctgaggctgtttcctcgtctgttaagtgtgaatatcaatacctgcctttcataatcaccagggaataaagtggaataatgttgataacagtgcttggcacctggaagtaggtggcagatgttaacgcccttcctcccttgcactgcgccccctgtgcctacctctagcattgtaacgaccacatagtattgaaatggccagtttacttgtctgccttcctttccaagaccgttggtgcctagaggactagaatcgtgtcctatttaactttgtgttcccaggtcctagctcaggagttggcaaataagaattaaatgtctgctacaccgaaacaaa

SEQ ID NO: 39 - DNA, 3536 bp, Homo sapiens
ggatcacgttggagggagcacacattgggagggtgggaacacaaggacaacgtagctcccactgaaacccacctgctgcccctacagcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctgaccctttcctatcaggagagatggcttgtcccctgccctgaagctggcagttcagtcccttattctgccctgttggaagccctgctgaaccctcaactattgactgctgcagacagcttatctccctaaccctgaaatgcggtgagcttgacttgactcccaaccctaccatgctccatcatactcaggtctccctactcctgccttagattcctcaataagatgctgtaactagcattttttgaatgcctctccctccgcatctcatctttctcttttcaatcaggcttttccaaagggttgtatacagactctgtgcactatttcacttgatattcattccccaattcactgcaaggagacctctactgtcaccgtttactctttcctaccctgacatccagaaacaatggcctccagtgcatacttctcaatctttgctttatggcctttccatcatagttgcccactccctctccttacttagcttccaggtcttaacttctctgactactcttgtcttcctctctcatcaatttctgcttcttcatggaatgctgaccttcattgctccatttgtagatttttgctcttctcagtttactcattgtcccctggaacaaatcactgacatctacaaccattaccatctcactaaataagactttctatccaataatgattgatacctcaaatgtaagatgcgtgatactcaacatttcatcgtccaccttcccaaccccaaacaattccatctcgtttcttcttggtaaatgatgctatgctttttccaaccaagccagaaacctgtgtcatcttttcaccccaccttcaatcaacaagtcctcaatcaacaagtcctactgactgcacatcttaaatatatctttatcagtccacaagtccttccaattatatttcccaagtatatctagaacttatccacttatatccccactgctactaccttagtttagggctatattctcttgaaaaaaagtgtccttacttcctgccaatccccaagtcatcttccagagtaaaatgcaaatcccatcaggccacttggatgaaaacccttcaaggattactggatagaattcaggctttcccctccagcccccaatcatagctcacaaaccttccttgctatttgttcttaagtaaaaaatcatttttcctcctccctccccaaaccccaaggaactctcactcttgctcaagctgttccgtccccttaccacccctgatacaactgccaggttaatttccagaattcttgcaagactcagttcagaagtcaccttctttcgtgaatgttttgattccctgaggctactttattttggtatggctgaaaaatcctagattttctaaacaaaacctgtttgaatcttggttctgatatggactaggagagagactgggtcaagtaagcttatctccctgaggctgtttcctcgtctgttaagtgtgaatatcaatacctgcctttcataatcaccagggaataaagtggaataatgttgataacagtgcttggcacctggaagtaggtggcagatgttaacgcccttcctcccttgcattgcgccccctgtgcctacctctagcattgtaacgaccacatagtattgaaatggccagtttacttgtctgccttcctttccaagaccgttggtgcctagaggactagaatcgtgtcctatttaactttgtgttcccaggtcctagctcaggagttggcaaataagaattaaatgtctgctacaccg

SEQ ID NO: 40 - DNA, 2427 bp, Homo sapiens
cccacgcgtccgcggacgcgtgggaagggcagaatgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggtctctttgatgtaacccgtggctgccatgagtcctgtctggatgaagaggtagagggccagggtttctgctctggtcctggctgggatcctgtaacaggctggggaacaccaacttcccagctttgctgaagactctactcaacccctgaccctttcctatcaggagagatggcttgtcccctgccctgaagctggcagttcagtcccttattctgccctgttggaagccctgctgaaccctcaactattgactgctgcagacagcttatctccctaaccctgaaatgctgtgagcttgacttgactcccaaccctaccatgctccatcatactcaggtctccctactcctgccttagattcctcaataagatgctgtaactagcattttttgaatgcctctccctccgcatctcatctttctcttttcaatcaggcttttccaaagggttgtatacagactctgtgcactatttcacttgatattcattccccaattcactgcaaggagacctctactgtcaccgtttactctttcctaccctgacatccagaaacaatggcctccagtgcatacttctcaatctttgctttatggcctttccatcatagttgcccactccctctccttacttagcttccaggtcttaacttctctgactactcttgtcttcctctctcatcaatttctgcttcttcatggaatgctgaccttcattgctccatttgtagatttttgctcttctcagtttactcattgtcccctggaacaaatcactgacatctacaaccattaccatctcactaaataagactttctatccaataatgattgatacctcaaatgtaaaaaa

SEQ ID NO: 41 - DNA, 2035 bp, Homo sapiens
agcagatccgcggaagggcagaatgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgaacacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctgaccctttcctatcaggagagatggcttgtcccctgccctgaagctggcagttcagtcccttattctgccctgttggaagccctgctgaaccctcaactattgactgctgcagacagcttatctccctaaccctgaaatgctgtgagcttgacttgactcccaaccctaccatgctccatcatactcaggtctccctactcctgccttagattcctcaataagatgctgtaactagcattttttgaatgcctctccctccgcatctcatctttctcttttcaatcaggcttttccaaagggtaaaaaaaaaaaaaaaaaaaaa

SEQ ID NO: 42 - DNA, 2514 bp, Homo sapiens
cggaagggcagaatgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacacgagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctgaccctttcctatcaggagagatggcttgtcccctgccctgaagctggcagttcagtcccttattctgccctgttggaagccctgctgaaccctcaactattgactgctgcagacagcttatctccctaaccctgaaatgctgtgagcttgacttgactcccaaccctaccatgctccatcatactcaggtctccctactcctgccttagattcctcaataagatgctgtaactagcattttttgaatgcctctccctccgcatctcatctttctcttttcaatcaggcttttccaaagggttgtatacagactctgtgcactatttcacttgatattcattccccaattcactgcaaggagacctctactgtcaccgtttactctttcctaccctgacatccagaaacaatggcctccagtgcatacttctcaatctttgctttatggcctttccatcatagttgcccactccctctccttacttagcttccaggtcttaacttctctgactactcttgtcttcctctctcatcaatttctgcttcttcatggaatgctgaccttcattgctccatttgtagatttttgctcttctcagtttactcattgtcccctggaacaaatcactgacatctacaaccattaccatctcactaaataagactttctatccaataatgattgatacctcaaatgtaagatgcgtgatactcaacatttcatcgtccaccttcccaaccccaaacaattccatctcgtttcttcttggtaaatgatgctatgctttttccaaccaaaaaaaaaaaaaaa

SEQ ID NO: 43 - DNA, 2479 bp, Macaca fascicularis
taccacatgacagcagatctgcggaagggcagaatgggactccaagcctgcctcctagggctctttgccctcatcctctctggcaaatgcagttacagcccggagcccgaccagcggagaacgctgcccccaggctgggtgtccttgggccgtgcggaccctgaggaagagctgagtctcacctttgccttgagacagcagaacctggaaagactctcggagctggtgcaggctgtgtcggatcccaactctcctcaatacggaaaatacttgaccctagagaatgtggctgatctggtgaggccatccccactgaccctccacatggtgcaaaaatggctcttggcagccggagcccagaagtgccattctgtgatcacacaggactttctgacttgctggctgagcatccggcaagcagagctgctactccctggggctgagtttcatcactatgtgggaggacctacagagacccatgttgtaaggtccccacgtccctaccagcttccacaggccttggccccccatgtggactttgtggggggactgcaccgttttcccccaacatcatccctgaggcaacgtcctgagccgcaggtgacagggactgtaggcctgcatttgggggtgaccccctctgtgatccgtaagcgatataacttgacctcacaagacgtgggctctggcaccagcaataacagccaagcctgtgcccagttcctggagcagtatttccatgactcagacctggctcagttcatgcgcctcttcggtggcaactttgcacatcaggcatcagtaacccgtgtggttggacaacagggccggggccgggccgggattgaggccagtctagatgtgcagtacctgatgagtgctggcgccaacatctccacctgggtctacagtagccctggccggcatgagggacaggagcccttcctgcagtggctcatgctgctcagtaacgagtcagccctgccacatgtgcatactgtgagctatggagatgaggaggactccctcagcagtgcctacatccagcgggtcaacacggagctcatgaaggctgctgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagttgtagccaagttcctgagctctagcccccaccttccaccatccggttacttcaatgccagtggccgtgcctatccagatgtggctgcactttctgatggctactgggtggtcaacaacagagtgcccattccatgggtgtctggaacctcggcctctactccagtgtttggggggctcctatccttgatcaatgagcacagaatcctcagtggccgcccccctcttggctttctcaacccaaggctctaccagcagcatggagcaggactctttgatgtaacccatggctgccatgcatcctgtctggatgaagaggtggagggccagggtttctgctctggccctggctgggatcctgtaacaggctggggaacacccaacttcccagctttgctgaagactctactcaacccctgacattttcctatcaggagagatggcttgtcccctgccctgaagctggcagttcagtcccttattctgccctattggaagccctgctgaaccctcaactattgactgctgcagacagcttatctgcctaaccctgaaatgctgtgagcttgacttgactcccaaccctaccatgctccattatactcaggtctccctactcctgccttagattcctcaataagatgctataactagcatttttttgaatgcatctccctctgcatctcatctttctcttttcaatcaggcttttccaaagggttgtgtacagactctgcactatttcacttcatattcactgcccaattcactgcaaggagacctctactctcaccttttaccttttcctaccctgacatccagaaacaatggcctgcagtgcatacttctcaatctttgctttacggcctttaaatcatagttgcccactccctctccttacttagcttccatggcttaacttctctgaccactcttgtcttcctccctcatcagcttctgctacttcatggaatgctgaccttcattgctccatttgtaggtttttgctcttctcagtttactcattgtcccctggaacaaatcactgacatctacaaccattaccatctcactaaataagactttctatccaatagtgattgatacctcaaatgtaagatgtgtgatattcgaacatttcatcttccaccttcccaaacccgaaacaagtcca

SEQ ID NO: 44 - DNA, 2539 bp, Macaca fascicularis
acatgacagcagatctgcggaagggcagaatgggactccaagcctgcctcctagggctctttgccctcatcctctctggcaaatgcagttacagcccggagcccgaccagcggagaacgctgcccccaggctgggtgtccttgggccgtgcggaccctgaggaagagctgagtctcacctttgccttgagacagcagaacctggaaagactctcggagctggtgcaggctgtgtcggatcccaactctcctcaatacggaaaatacttgaccctagagaatgtggctgatctggtgaggccatccccactgaccctccacatggtgcaaaaatggctcttggcagccggagcccagaagtgccattctgtgatcacacaggactttctgacttgctggctgagcatccggcaagcagagctgctactccctggggctgagtttcatcactatgtgggaggacctacagagacccatgttgtaaggtccccacgtccctaccagcttccacaggccttggccccccatgtggactttgtggggggactgcaccgttttcccccaacatcatccctgaggcaacgtcctgagccgcaggtgacagggactgtaggcctgcatttgggggtgaccccctctgtgatccgtaagcgatataacttaacctcacaagacgtgggctctggcaccagcaataacagccaagcctgtgcccagttcctggagcagtatttccatgactcagacctggctcagttcatgcgcctcttcggtggcaactttgcacatcaggcatcagtaacccgtgtggttggacaacagggccggggccgggccgggattgaggccagtctagatgtgcagtacctgatgagtgctggcgccaacatctccacctgggtctacagtagccctggccggcatgagggacaggagcccttcctgcagtggctcatgctgctcagtaacgagtcagccctgccacatgtgcatactgtgagctatggagatgaggaggactccctcagcagtgcctacatccagcgggtcaacacggagctcatgaaggctgctgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtagccaagttcctgagctctagcccccaccttccaccatccggttacttcaatgccagtggccgtgcctatccagatgtggctgcactttctgatggctactgggtggtcagcaacagagtgcccattccatgggtgtccggaacctcggcctctactccagtgtttggggggctcctatccttgatcaatgagcacagaatcctcagtggccgcccccctcttggctttctcaacccaaggctctaccagcagcatggggcaggactctttgatgtaacccatggctgccatgcatcctgtctggatgaagaggtggagggccagggtttctgctctggccctggctgggatcctgtaacaggctggggaacacccaacttcccagctttgctgaagactctactcaacccctgacattttcctatcaggagagatggcttgtcccctgccctgaagctggcagttcagtcccttattctgccctattggaagccctgctgaaccctcaactactgactgctgcagacagcttatctgcctaaccctgaaatgctgtgagcttgacttgactcccaaccctaccatgctccattatactcaggtctccctactcctgccttagattcctcaataagatgctataactagcatttttttgaatgcatctccctctgcatctcatctttctcttttcaatcaggcttttccaaagggttgtgtacagactctgcactatttcacttcatattcactgcccaattcactgcaaggagacctctactctcaccttttaccttttcctaccctgacatccagaaacaatggcctgcagtgcatacttctcaatctttgctttatggcctttccatcatagttgcccactccctctccttacttagcttccatggcttaacttctctgaccactcttgtcttcctccctcatcagcttctgctacttcatggaatgctgaccttcattgctccatttgtaggtttttgctcttctcagtttactcattgtcccctggaacaaatcactgacatctacaaccattaccatctcactaaataagactttctatccaatagtgattgatacctcaaatgtaagatgtgtgatattcaacatttcatcttccaccttcccaaacccaaacaattccatctctggtttcttcttggtaaatgatgctatgctttttccaaccaaaaaaaaaaaaaaaaaaaaaa

SEQ ID NO: 45 - DNA, 1755 bp, Mus musculus
gaattcggatccatgtgggactccaagcccgcctcctagggctccttgctctcgtcatcgccggcaaatgcacttacaaccctgagccggaccagcggtggatgctgcctccaggctgggtgtccctgggccgcgtggatcccgaggaagagctgagtctcacttttgcgctgaaacagcggaacctggaaagactctcggagctggtgcaggctgtgtcggatcctagctctcctcaatatggaaagtacctaaccctggaggatgtagctgagctggttcaaccatcacccctgaccctcctcactgtccaaaagtggctctcagcagctggagcccggaactgcgattcagtgaccacccaggactttctgacttgctggctgagtgtccgacaggctgagctgctgctcccaggagctgagtttcatcgctatgtagggggacctacaaagacccatgttataaggtccccacatccctaccagcttccccaggccttggcccctcatgtggattttgtgggggggctgcaccgtttccccccttcatctccaagacaacgtccagaaccacaacaggtaggaactgttagcctgcacttgggagtgactccgtctgtgctccgtcagcgatacaacctgacagccaaagatgtgggctcaggcaccaccaacaatagccaggcctgtgcccagttcctggaacagtacttccataactcggatctgactgagttcatgcgcctattcggtggcagttttacacaccaggcctcagtagcaaaagttgttggaaagcaagggcgaggccgagctgggatcgaggccagtctagatgtggaatacctgatgagtgctggtgccaatatctccacttgggtctacagtagccctggccgccatgaggcacaggagcccttcttacaatggctcctgcttcttagcaatgagtcatctttgccacatgtacatactgtgagttacggagacgatgaagactccctcagcagcatctacatccagagagtcaacactgagttcatgaaggctgctgctcggggtctcaccctcctttttgcctcaggtgacactggagctgggtgttggtctgtctccggaagacacaagttccgccctagcttccctgcttccagcccctatgttactacagttggaggaacctccttcaagaatcctttcctcatcacagatgaagtagttgactatatcagtggtggaggcttcagcaatgttttcccacggcctccctaccaggaggaagcagtggcccagttcttgaaatccagctctcatctaccaccatccagttacttcaatgctagtggccgtgcctacccagatgttgccgcactatctgatggctactgggtggtcagcaacatggtccccattccatgggtatctggaacctcggcctctactccagtgtttgggggaattttatccttgataaatgagcacagaatcctcaatggccgccctcctcttggctttctcaaccccaggctctatcagcagcatgggacaggactctttgatgtaacccacggctgccatgagtcctgtctgaatgaagaagtggagggtcagggtttctgctctggtcctggctgggatcctgtgacaggttggggaacacccaacttcccagccctactgaagaccctgctcaaccttgaccctttcgtgccatgacgagaaagcagaactgttccctgtactaaaagggaag

SEQ ID NO: 46 - DNA, 6108 bp, Mus musculus
aaatgggactccaagcccggtgagaaactgaggtggggagggtagctaagggggattacattagagggattacatgttgtagaaggagtacaatgagggttcggctcctgatgaaacccacctgctgtccctgcagcctcctagggctccttgctctcgtcatcgccggcaaatgcacttacaaccctgagccggaccagcggtggatgtgagttgatttagtactacaggtgcctgtctctgcagacgcttggtcctagctttcctcatcctcaactgctcctagtacccactctgtagccttattcgacaccccctcggcttaatctctggtcatttatctcctatgatccttatctgcatctaggattcgacccaaagtctcagttcttaatcccaaactttccatgatctggatttctcgttatggcctaatcaaatcatgtccctgaccctgtaggctgcctccaggctgggtgtccctgggccgcgtggatcccgaggaagagctgagtctcacttttgcgctgaaacagcggaacctggaaagactctcggagctggtgcaggctgtgtcggatcctagctctcctcaatatggtgcctatcagttggggcaggaagtgggaggcaggaccaggcaggcactggtggtgcagatcatgctagggccatgtttctggtatgacagacaaatgacaataactgccatattcatacacacacacacacacacacacacacacacacacacacaccaaaatttccacctgagttcttcagtcttaaaactaagaacaaatctaagcctagtagcatgaggtaataatcctagctacttggactaaaacaggacgattttgaatccaaggcatgcctgggcagtgtagcaatattgtcaacattaaaaccaattttttttagaggcctagctagctcattgatagggtgggtgcttacggagcatatgaaaaaccctaggttcaagtgccaggaagatggtgggtgtggggtagctagctgtgtttaagaacacatgattcataatcagatctagattgaaatgttcacttaccctaccagctctcaaatggaggactagcacttagtgtgtgtgcatgatcaagtcatggaccttaccccactctcatagccactgccacaccctcctctgttgctagttcaagtcctaaattgtctttggattaatccactctggccattcctcttgctaccaactcagttgtttattctctttctggattactgcccagtacccggactggtttgaagcttcctctccttcaccccactccccagaacagtcccttctctattctagagtattctctatggtgctcaggctggagtgaatccttcccctgcttaaagtcctgtatggtgaggcaagggtgtgtgtgtgtgtgtgtgtgtgtagaatggaggcgagaggaggggggggcatccttagagcacccaagaagctcctcatatgcaggaaagaatgactcaccgttatgttgtttaaaatcaacaaggatgaagagctgagctaaattagaaggttctgtataactggctccaaaagtttggggaaatctttttgaagataagcttaaaatgggatattgaaagtttactaacgtccttgagtttttgtgaccatggtaacacatgctgtgcatttttcctggccatgacgtttcatgtctctcgagccttttcttccctttgactctcactatgcctagccctacacagtcctcctcgggaagttttgatgtcctctgattgaggtccagacagacaacccaggaaagcctcaaatgaggacgagagcctcttctgaatccctgcaggaaagtacctaaccctggaggatgtagctgagctggttcaaccatcacccctgaccctcctcactgtccaaaagtggctctcagcagctggagcccggaactgcgattcagtgaccacccaggactttctgacttgctggctgagtgtccggtgagaagtaatgatttccccatagaatccattgtccctacaaggagacaaaataccccaattgggggagtttaaagcgtgctgggagcctgtgggtataggtaatgcatacggaaatgatggcaggctgctaagtggggctcaggccaactcttctactccttcccatggtttctgttttcttacttccagacaggctgagctgctgctcccaggagctgagtttcatcgctatgtagggggacctacaaagacccatgttataaggtccccacatccctaccagcttccccaggccttggcccctcatgtggattttggtaaacccaatggagttggtggaagttgtgggagggaggtctacaggctgaagaattttagatgccaaaagaggcatagaatctttccagtagagaagtgggtggtagtgtctgtaagacctccttaagcctgacccctttccacagtgggggggctgcaccgtttccccccttcatctccaagacaacgtccagaaccacaacaggtaggaactgttagcctgcacttgggagtgactccgtctgtgctccgtcagcgatacaacctgacagccaaagatgtgggctcaggcaccaccaacaatagccaggcctgtgcccaggtgagccatgtaaagccctgcggtcatcacaacctcctcaagatatctttagtgcctcactaccctgggcctctctctgatccacaattcctgacccggatgtttccacagttcctggaacagtacttccataactcggatctgactgagttcatgcgcctattcggtggcagttttacacaccaggcctcagtagcaaaagttgttggaaagcaagggcgaggccgagctgggatcgaggccagtctagatgtggaatacctgatgagtgctggtgccaatatctccacttgggtctacagtagccctggtattgctaagagaattagttgggggatgggaaaatgggttggagtagacttttggtctctgcttcatttcatcaaggggatgccatgggctgaagggagattctagcaaccatccaatggccattcatatcccttcttttaaaacaattcaggccgccatgaggcacaggagcccttcttacaatggctcctgcttcttagcaatgagtcatctttgccacatgtacatactgtgagttacggagacgatgaagactccctcagcagcatctacatccagagagtcaacactgagttcatgaaggctgctgctcggggtctcaccctcctttttgcctcaggtaaccttctaccataaatttaagacttcccacctacccaagcggcagactttatcccaaccgacccttcagcctggtttctgactcataataggaactcagagccttaacagggtctgctgatttgacctgagtactctgaggtggcatatacagttcttctctggtatatagaagcctacaccccaagttcttcacaactaatctgaatacttactatgtctttcccaggtgacactggagctgggtgttggtctgtctccggaagacacaagttccgccctagcttccctgcttccaggtaagtaccccactctttcacttgtgacagaggccaccaggagctgctgtggctcagccttcagcattacctgttgtgtttgctgtgctcccttttcttcctgcgtatcccaggctgcgagagcagattatggcgtttcttttccttagttgctagggtttgttgcttgtttttcatgtagaaaagtatatacaattaactccagccatgtcttgagagctcccaatctatcaataaactctgtatacaggcttctatagtcttactcccttttccagtaagacccagaccattcccacccacctccacacatcttggaggtcacccattgtcttagtggggtttttattgtattgctgcgatgaaacaccgtgacaaaaaaacaaaacaaaacaaaacaaaacaaaaacagttgaggaggaaagggtttatttggcttacacttccagatcacatccgtcactgaaggaaatcaggacagaaactcaagcagggctggaacctggaagcagaagctgatgaagaggccagggaatggtgctgcttactggcttgcttcccatggcttgttcagcctgccatcttatagaacccacgaccatcagcccagggatgccaccctccacaatgggctgggccctccctcattgatcactaattgagaaaatgtcctacagctggatctcatggaggcatttccttaactgagcttcctttgtctctgatgactcttgtatcaagttgacaacacaaaactaaccagcaagtacattcactatctgaatactgtcttctcctcagcccctatgttactacagttggaggaacctccttcaagaatcctttcctcatcacagatgaagtagttgactatatcagtggtggaggcttcagcaatgttttcccacggcctccctaccaggtttgtggatattcctgtggatatctggaggttgaaggtgatgggtggggctcagtcctgcagcttgctgagcaggctgctggccaatactcatactcagaaatgtccttcaggaggaagcagtggcccagttcttgaaatccagctctcatctaccaccatccagttacttcaatgctagtggccgtgcctacccagatgttgccgcactatctgatggctactgggtggtcagcaacatggtccccattccatgggtatctggaacctcggtaagaatcagctctgctctaaacgctctactcaggaactaccctcgctcctccacctacacaatctaaacgctctactcaggaactaccctcgctcctccacctacacaatctaaacgctctactcaggaactaccctcgctcctccacctacacaatctaaacgctctactcaggaactaccctcgctcctccacctacacaatcttgaacccagaacccccgactccttggagactcctgatcattgcaagcatcatcccttagaagtccaatccctctaaaaccctaacctattcttgcatcttcatcttgcaggcctctactccagtgtttgggggaattttatccttgataaatgagcacagaatcctcaatggccgccctcctcttggctttctcaaccccaggctctatcagcagcatgggacaggactctttgatgtgagtattggaggaaagagtgtggatgttgtcataggatatgagaagggctctggtgaacttcggcattttcactatctatgattgcctctgtgatatgactataaataaggtgcagtctaggagctggtaccagcaggcccagacctgatgccatcatctcctcccaggtaacccacggctgccatgagtcctgtctgaatgaagaagtggagggtcagggtttctgctctggtcctggctgggatcctgtgacaggttggggaacacccaacttcccagccctactgaagaccctgctcaacccttgaccctttcgtgccatgacgagaaagcagaactgttccctgtactaaaagggaaggctcagtttcttgttattcctcgatagaagccctgctgaactcctgttgcctgctgcagatagcttctccctaaccctcagatgctgtgaacaggactcaactctcaatcctactgtgtgccatcaaactcaggtctccaaacttctacttcaagatcctcaacaagatgctataaccagcatattttgtctcaccccaaccccatctctccttcctctttccagcttgagatgtgaaagcagggcaagaaggttcagtcttccattactgacactagcaggtccacccaacgcttaccacctctgcactgaccgtacactctatttctcttcgggtttgcttttccgttcactgaagtgagacctttgactaatcgttttgtctttcttctctcggcactgaagtacaatggtctccccaatgttttatccagttatacccttttcagtgtttgttttatgggttttcttatttaagaacaggttgtcaaaaaaccatt

SEQ ID NO: 47 - DNA, 2270 bp, Mus musculus
atgactacagatcagcctgaaagccaaaatgggactccaagcccgcctcctagggctccttgctctcgtcatcgccggcaaatgcacttacaaccctgagccggaccagcggtggatgctgcctccaggctgggtgtccctgggccgcgtggatcccgaggaagagctgagtctcacttttgcgctgaaacagcggaacctggaaagactctcggagctggtgcaggctgtgtcggatcctagctctcctcaatatggaaagtacctaaccctggaggatgtagctgagctggttcaaccatcacccctgaccctcctcactgtccaaaagtggctctcagcagctggagcccggaactgcgattcagtgaccacccaggactttctgacttgctggctgagtgtccgacaggctgagctgctgctcccaggagctgagtttcatcgctatgtagggggacctacaaagacccatgttataaggtccccacatccctaccagcttccccaggccttggcccctcatgtggattttgtgggggggctgcaccgtttccccccttcatctccaagacaacgtccagaaccacaacaggtaggaactgttagcctgcacttgggagtgactccgtctgtgctccgtcagcgatacaacctgacagccaaagatgtgggctcaggcaccaccaacaatagccaggcctgtgcccagttcctggaacagtacttccataactcggatctgactgagttcatgcgcctattcggtggcagttttacacaccaggcctcagtagcaaaagttgttggaaagcaagggcgaggccgagctgggatcgaggccagtctagatgtggaatacctgatgagtgctggtgccaatatctccacttgggtctacagtagccctggccgccatgaggcacaggagcccttcttacaatggctcctgcttcttagcaatgagtcatctttgccacatgtacatactgtgagttacggagacgatgaagactccctcagcagcatctacatccagagagtcaacactgagttcatgaaggctgctgctcggggtctcaccctcctttttgcctcaggtgacactggagctgggtgttggtctgtctccggaagacacaagttccgccctagcttccctgcttccagcccctatgttactacagttggaggaacctccttcaagaatcctttcctcatcacagatgaagtagttgactatatcagtggtggaggcttcagcaatgttttcccacggcctccctaccaggaggaagcagtggcccagttcttgaaatccagctctcatctaccaccatccagttacttcaatgctagtggccgtgcctacccagatgttgccgcactatctgatggctactgggtggtcagcaacatggtccccattccatgggtatctggaacctcggcctctactccagtgtttgggggaattttatccttgataaatgagcacagaatcctcaatggccgccctcctcttggctttctcaaccccaggctctatcagcagcatgggacaggactctttgatgtaacccacggctgccatgagtcctgtctgaatgaagaagtggagggtcagggtttctgctctggtcctggctgggatcctgtgacaggttggggaacacccaacttcccagccctactgaagaccctgctcaacccttgaccctttcgtgccatgacgagaaagcagaactgttccctgtactaaaagggaaggctcagtttcttgttattcctcgatagaagccctgctgaactcctgttgcctgctgcagatagcttctccctaaccctcagatgctgtgaacaggactcaactctcaatcctactgtgtgccatcaaactcaggtctccaaacttctacttcaagatcctcaacaagatgctataaccagcatattttgtctcaccccaaccccatctctccttcctctttccagcttgagatgtgaaagcagggcaagaaggttcagtcttccattactgacactagcaggtccacccaacgcttaccacctctgcactgaccgtacactctatttctcttcgggtttgcttttccgttcactgaagtgagacctttgactaatcgttttgtctttcttctctcggcactgaagtacaatggtctccccaatgttttatccagttatacccttttcagtgtttgttttatgggttttcttatttaagaacaggttgtcaaaaaaccatt

SEQ ID NO: 48 - DNA, 2281 bp, Mus musculus
ggacttatcacatgactacagatcagcctgaaagccaaaatgggactccaagcccgcctcctagggctccttgctctcgtcatcgccggcaaatgcacttacaaccctgagccggaccagcggtggatgctgcctccaggctgggtgtccctgggccgcgtggatcccgaggaagagctgagtctcacttttgcgctgaaacagcggaacctggaaagactctcggagctggtgcaggctgtgtcggatcctagctctcctcaatatggaaagtacctaaccctggaggatgtagctgagctggttcaaccatcacccctgaccctcctcactgtccaaaagtggctctcagcagctggagcccggaactgcgattcagtgaccacccaggactttctgacttgctggctgagtgtccgacaggctgagctgctgctcccaggagctgagtttcatcgctatgtagggggacctacaaagacccatgttataaggtccccacatccctaccagcttccccaggccttggcccctcatgtggattttgtgggggggctgcaccgtttccccccttcatctccaagacaacgtccagaaccacaacaggtaggaactgttagcctgcacttgggagtgactccgtctgtgctccgtcagcgatacaacctgacagccaaagatgtgggctcaggcaccaccaacaatagccaggcctgtgcccagttcctggaacagtacttccataactcggatctgactgagttcatgcgcctattcggtggcagttttacacaccaggcctcagtagcaaaagttgttggaaagcaagggcgaggccgagctgggatcgaggccagtctagatgtggaatacctgatgagtgctggtgccaatatctccacttgggtctacagtagccctggccgccatgaggcacaggagcccttcttacaatggctcctgcttcttagcaatgagtcatctttgccacatgtacatactgtgagttacggagacgatgaagactccctcagcagcatctacatccagagagtcaacactgagttcatgaaggctgctgctcggggtctcaccctcctttttgcctcaggtgacactggagctgggtgttggtctgtctccggaagacacaagttccgccctagcttccctgcttccagcccctatgttactacagttggaggaacctccttcaagaatcctttcctcatcacagatgaagtagttgactatatcagtggtggaggcttcagcaatgttttcccacggcctccctaccaggaggaagcagtggcccagttcttgaaatccagctctcatctaccaccatccagttacttcaatgctagtggccgtgcctacccagatgttgccgcactatctgatggctactgggtggtcagcaacatggtccccattccatgggtatctggaacctcggcctctactccagtgtttgggggaattttatccttgataaatgagcacagaatcctcaatggccgccctcctcttggctttctcaaccccaggctctatcagcagcatgggacaggactctttgatgtaacccacggctgccatgagtcctgtctgaatgaagaagtggagggtcagggtttctgctctggtcctggctgggatcctgtgacaggttggggaacacccaacttcccagccctactgaagaccctgctcaacccttgaccctttcgtgccatgacgagaaagcagaactgttccctgtactaaaagggaaggctcagtttcttgttattcctcgatagaagccctgctgaactcctgttgcctgctgcagatagcttctccctaaccctcagatgctgtgaacaggactcaactctcaatcctactgtgtgccatcaaactcaggtctccaaacttctacttcaagatcctcaacaagatgctataaccagcatattttgtctcaccccaaccccatctctccttcctctttccagcttgagatgtgaaagcagggcaagaaggttcagtcttccattactgacactagcaggtccacccaacgcttaccacctctgcactgaccgtacactctatttctcttcgggtttgcttttccgttcactgaagtgagacctttgactaatcgttttgtctttcttctctcggcactgaagtacaatggtctccccaatgttttatccagttatacccttttcagtgtttgttttatgggttttcttatttaagaacaggttgtcaaaaaaccatt

SEQ ID NO: 49 - DNA, 3494 bp, Mus Musculus
gagatcagcctgaaagccaaaatgggactccaagcccgcctcctagggctccttgctctcgtcatcgccggcaaatgcacttacaaccctgagccggaccagcggtggatgctgcctccaggctgggtgtccctgggccgcgtggatcccgaggaagagctgagtctcacttttgcgctgaaacagcggaacctggaaagactctcggagctggtgcaggctgtgtcggatcctagctctcctcaatatggaaagtacctaaccctggaggatgtagctgagctggttcaaccatcacccctgaccctcctcactgtccaaaagtggctctcagcagctggagcccggaactgcgattcagtgaccacccaggactttctgacttgctggctgagtgtccgacaggctgagctgctgctcccaggagctgagtttcatcgctatgtagggggacctacaaagacccatgttataaggtccccacatccctaccagcttccccaggccttggcccctcatgtggattttgtgggggggctgcaccgtttccccccttcatctccaagacaacgtccagaaccacaacaggtaggaactgttagcctgcacttgggagtgactccgtctgtgctccgtcagcgatacaacctgacagccaaagatgtgggctcaggcaccaccaacaatagccaggcctgtgcccagttcctggaacagtacttccataactcggatctgactgagttcatgcgcctattcggtggcagttttacacaccaggcctcagtagcaaaagttgttggaaagcaagggcgaggccgagctgggatcgaggccagtctagatgtggaatacctgatgagtgctggtgccaatatctccacttgggtctacagtagccctggccgccatgaggcacaggagcccttcttacaatggctcctgcttcttagcaatgagtcatctttgccacatgtacatactgtgagttacggagacgatgaagactccctcagcagcatctacatccagagagtcaacactgagttcatgaaggctgctgctcggggtctcaccctcctttttgcctcaggtgacactggagctgggtgttggtctgtctccggaagacacaagttccgccctagcttccctgcttccagcccctatgttactacagttggaggaacctccttcaagaatcctttcctcatcacagatgaagtagttgactatatcagtggtggaggcttcagcaatgttttcccacggcctccctaccaggaggaagcagtggcccagttcttgaaatccagctctcatctaccaccatccagttacttcaatgctagtggccgtgcctacccagatgttgccgcactatctgatggctactgggtggtcagcaacatggtccccattccatgggtatctggaacctcggcctctactccagtgtttgggggaattttatccttgataaatgagcacagaatcctcaatggccgccctcctcttggctttctcaaccccaggctctatcagcagcatgggacaggactctttgatgtaacccacggctgccatgagtcctgtctgaatgaagaagtggagggtcagggtttctgctctggtcctggctgggatcctgtgacaggttggggaacacccaacttcccagccctattgaagaccctgctcaacccttgaccctttcgtgccatgacgagaaagcagaactgttccctgtactaaaagggaaggctcagtttcttgttattcctcgatagaagccctgctgaactcctgttgcctgctgcagatagcttctccctaaccctcagatgctgtgaacaggactcaactctcaatcctactgtgtgccatcaaactcaggtctccaaacttctacttcaagatcctcaacaagatgctataaccagcatattttgtctcaccccaaccccatctctccttcctctttccagcttgagatgtgaaagcagggcaagaaggttcagtcttccattactggcactagcaggtccacccaacgcttaccacctctgcactgaccgtacactctatttctcttcgggtttgcttttccgttcactgaagtgagacctttgactaatcgttttgtctttcttctctcggcactgaagtacaatggtctccccaatgttttatccagttatacccttttcagtgtttgttttatgggttttcttatttaagaacaggttgtcaaaaaaccattaaaaaaaaaaagaaaaagaacacattggctgcaatctatttaactatataactattctaaggaaagttaaaaattgaaaacttaaaatgtttgaaatgttctcattggcaaaattcctcaacaaaataaataggtcattacaaattttgctttaaatttttgcttgagtgatttttttttttgtaaagtgttttaaaatttacattttattttccccttctcagctacaccaaagcaacattcaggttttaatttcaactgtcagacattacaaacatctagcttctgtgaacctgggtgtttgttctttccatcagtttcccatttatcctttttcctcagtgacccccctctgaccacactaatcagcccccttttctgctgactccagggatgctgtgttcattgttccattcttagtttcttgctcttatcattttattatcatcccttgacagatcactgacatctatacccacagtgagtgatacctcaaatgtgagatacctcgttacttcatctccctccagcccagacctaaactatttcatctcttaatctatgatataatgcctctttcaaacaagccagaaacctataactcttaactctcatctttttcacctccttactaacttcaaaaagttttcttgactgcctctcaagtatatctttatcggctagtgtggtgatgcacacctttaatcccagcactcaggagacagaggccaggcaagtctctgagtttgaggccatcctggtctatataaagagttccagacaagccaggcctacatagtcagaccatgtctcaaaaatacacatatgtgcacacacacacatgcacaaaatactgcattatctttgagcccatgttcttttttcttctaaactgcaggaagcacttggggagaggaaaggcttccgaagcccctcatcttccaaaccaagcagtctatgtatttgtgcaaacccttcaaagattactgggtaaaagtcagagacattgaaacttgccttcaaaatcgggaaataaacattgctaatgtctcacacttggaattaagcaatgaatgttagtttccctttcttctttgcactgcacctacatgtagctgggaaacaatcatacagtattgaaattgtcagtttgtttgccttccttttccagacagtcggtgctagtgaactagaatctgattcttaactttgtattcctaggtcccagccc

SEQ ID NO: 50 - DNA, 3319 bp, Mus musculus
gcgcttgaaagccaaaatgggactccaagcccgcctcctagggctccttgctctcgtcatcgccggcaaatgcacttacaaccctgagccggaccagcggtggatgctgcctccaggctgggtgtccctgggccgcgtggatcccgaggaagagctgagtctcacttttgcgctgaaacagcggaacctggaaagactctcggagctggtgcaggctgtgtcggatcctagctctcctcaatatggaaagtacctaaccctggaggatgtagctgagctggttcaaccatcacccctgaccctcctcactgtccaaaagtggctctcagcagctggagcccggaactgcgattcagtgaccacccaggactttctgacttgctggctgagtgtccgacaggctgagctgctgctcccaggagctgagtttcatcgctatgtagggggacctacaaagacccatgttataaggtccccacatccctaccagcttccccaggccttggcccctcatgtggattttgtgggggggctgcaccgtttccccccttcatctccaagacaacgtccagaaccacaacaggtaggaactgttagcctgcacttgggagtgactccgtctgtgctccgtcagcgatacaacctgacagccaaagatgtgggctcaggcaccaccaacaatagccaggcctgtgcccagttcctggaacagtacttccataactcggatctgactgagttcatgcgcctattcggtggcagttttacacaccaggcctcagtagcaaaagttgttggaaagcaagggcgaggccgagctgggatcgaggccagtctagatgtggaatacctgatgagtgctggtgccaatatctccacttgggtctacagtagccctggccgccatgaggcacaggagcccttcttacaatggctcctgcttcttagcaatgagtcatctttgccacatgtacatactgtgagttacggagacgatgaagactccctcagcagcatctacatccagagagtcaacactgagttcatgaaggctgctgctcggggtctcaccctcctttttgcctcaggtgacactggagctgggtgttggtctgtctccggaagacacaagttccgccctagcttccctgcttccagcccctatgttactacagttggaggaacctccttcaagaatcctttcctcatcacagatgaagtagttgactatatcagtggtggaggcttcagcaatgttttcccacggcctccctaccaggaggaagcagtggcccagttcttgaaatccagctctcatctaccaccatccagttacttcaatgctagtggccgtgcctacccagatgttgccgcactatctgatggctactgggtggtcagcaacatggtccccattccatgggtatctggaacctcggcctctactccagtgtttgggggaattttatccttgataaatgagcacagaatcctcaatggccgccctcctcttggctttctcaaccccaggctctatcagcagcatgggacaggactctttgatgtaacccacggctgccatgagtcctgtctgaatgaagaagtggagggtcagggtttctgctctggtcctggctgggatcctgtgacaggttggggaacacccaacttcccagccctactgaagaccctgctcaacccttgaccctttcgtgccatgacgagaaagcagaactgttccctgtactaaaagggaaggctcagtttcttgttattcctcgatagaagccctgctgaactcctgttgcctgctgcagatagcttctccctaaccctcagatgctgtgaacaggactcaactctcaatcctactgtgtgccatcaaactcaggtctccaaacttctacttcaagatcctcaacaagatgctataaccagcatattttgtctcaccccaaccccatctctccttcctctttccagcttgagatgtgaaagcagggcaagaaggttcagtcttccattactgacactagcaggtccacccaacgcttaccacctctgcactgaccgtacactctatttctcttcgggtttgcttttccgttcactgaagtgagacctttgactaatcgttttgtctttcttctctcggcactgaagtacaatggtctccccaatgttttatccagttatacccttttcagtgtttgttttatgggttttcttatttaagaacaggttgtcaaaaaaccattaaaaaaaaaaaagaaaaagaacacattggctgcaatctatttaactatataactattctaaggaaagttaaaaattgaaaacttaaaatgtttgaaatgttctcattggcaaaattcctcaacaaaataaataggtcattacaaattttgctttaaatttttgcttgagtgatttttttttttgtaaagtgttttaaaatttacattttattttccccttctcagctacaccaaagcaacattcaggttttaatttcaactgtcagacattacaaacatctagcttctgtgaacctgggtgtttgttctttccatcagtttcccatttatcctttttcctcagtgacccccctctgaccacactaatcagcccccttttctgctgactccagggatgctgtgttcattgttccattcttagtttcttgctcttatcattttattatcatcccttgacagatcactgacatctatacccacagtgagtgatacctcaaatgtgagatacctcgttacttcatctccctccagcccagacctaaactatttcatctcttaatctatgatataatgcctctttcaaacaagccagaaacctataactcttaactctcatctttttcacctccttactaacttcaaaaagttttcttgactgcctctcaagtatatctttatcggctagtgtggtgatgcacacctttaatcccagcactcaggagacagaggccaggcaagtctctgagtttgaggccatcctggtctatataaagagttccagacaagccaggcctacatagtcagaccatgtctcaaaaatacacatatgtgcacacacacacatgcacaaaatactgcattatctttgagcccatgttcttttttcttctaaactgcaggaagcacttggggagaggaaaggcttccgaagcccctcatcttccaaaccaagcagtctatgtatttgtgcaaacccttcaaagattactgggtaaaagtcagagacattgaaacttgccttcaaaatcgggaaataaacattgctaatgtctcacacttgg

SEQ ID NO: 51 - DNA, 2279 bp, Mus musculus
cggacgcgtggggaaagccaaaatgggactccaagcccgcctcctagggctccttgctctcgtcatcgccggcaaatgcacttacaaccctgagccggaccagcggtggatgctgcctccaggctgggtgtccctgggccgcgtggatcccgaggaagagctgagtctcacttttgcgctgaaacagcggaacctggaaagactctcggagctggtgcaggctgtgtcggatcctagctctcctcaatatggaaagtacctaaccctggaggatgtagctgagctggttcaaccatcacccctgaccctcctcactgtccaaaagtggctctcagcagctggagcccggaactgcgattcagtgaccacccaggactttctgacttgctggctgagtgtccgacaggctgagctgctgctcccaggagctgagtttcatcgctatgtagggggacctacaaagacccatgttataaggtccccacatccctaccagcttccccaggccttggcccctcatgtggattttgtgggggggctgcaccgtttccccccttcatctccaagacaacgtccagaaccacaacaggtaggaactgttagcctgcacttgggagtgactccgtctgtgctccgtcagcgatacaacctgacagccaaagatgtgggctcaggcaccaccaacaatagccaggcctgtgcccagttcctggaacagtacttccataactcggatctgactgagttcatgcgcctattcggtggcagttttacacaccaggcctcagtagcaaaagttgttggaaagcaagggcgaggccgagctgggatcgaggccagtctagatgtggaatacctgatgagtgctggtgccaatatctccacttgggtctacagtagccctggccgccatgaggcacaggagcccttcttacaatggctcctgcttcttagcaatgagtcatctttgccacatgtacatactgtgagttacggagacgatgaagactccctcagcagcatctacatccagagagtcaacactgagttcatgaaggctgctgctcggggtctcaccctcctttttgcctcaggtgacactggagctgggtgttggtctgtctccggaagacacaagttccgccctagcttccctgcttccagcccctatgttactacagttggaggaacctccttcaagaatcctttcctcatcacagatgaagtagttgactatatcagtggtggaggcttcagcaatgttttcccacggcctccctaccaggaggaagcagtggcccagttcttgaaatccagctctcatctaccaccatccagttacttcaatgctagtggccgtgcctacccagatgttgccgcactatctgatggctactgggtggtcagcaacatggtccccattccatgggtatctggaacctcggcctctactccagtgtttgggggaattttatccttgataaatgagcacagaatcctcaatggccgccctcctcttggctttctcaaccccaggctctatcagcagcatgggacaggactctttgatgtaacccacggctgccatgagtcctgtctgaatgaagaagtggagggtcagggtttctgctctggtcctggctgggatcctgtgacaggttggggaacacccaacttcccagccctactgaagaccctgctcaacccttgaccctttcgtgccatgacgagaaagcagaactgttccctgtactaaaagggaaggctcagtttcttgttattcctcgatagaagccctgctgaactcctgttgcctgctgcagatagcttctccctaaccctcagatgctgtgaacaggactcaactctcaatcctactgtgtgccatcaaactcaggtctccaaacttctacttcaagatcctcaacaagatgctataaccagcatattttgtctcaccccaaccccatctctccttcctctttccagcttgagatgtgaaagcagggcaagaaggttcagtcttccattactgacactagcaggtccacccaacgcttaccacctctgcactgaccgtacactctatttctcttcgggtttgcttttccgttcactgaagtgagacctttgactaatcgttttgtctttcttctctcggcactgaagtacaatggtctccccaatgttttatccagttatacccttttcagtgtttgttttatgggttttcttatttaagaacaggttgtcaaaaaaccattaaaaaaaaaaaaaaa

SEQ ID NO: 52 - DNA, 1692 bp, Pan troglodytes
atgggactccaagcctgcctcctagggctctttgccctcatcctctctggcaaatgcagctacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccgcacggtgcaaaaatggctcttggcagccggagcccggaagtgccattctgtgatcacacaggactttctgacttgctggctgagcatccgacaagcagagctgctgctccctggggctgagtttcatcactatgtgggaggacctacggagacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgaatcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaaccaagttcctgagctctagcccccacctgccaccatccagttacttcaatgccagtggccgtgcctacccagatgtggctgcactttccgatggctactgggtggtcagcaacagagtgcccattccatgggtgtccggaacctcggcctctactccagtgtttggggggatcctatccttgatcaatgagcacaggatccttagtggccgccctcctcttggctttctcaacccaaggctctaccagcagcatggggcaggactctttgatgtaacccgtggctgccatgagtcctgtctggatgaagaggtagagggccagggtttctgctctggccctggctgggatcctgtaacaggctggggaacacccaacttcccagctttgctgaagactctactcaacccctga

SEQ ID NO: 53 - DNA, 3484 bp, Pongo pygmaeus
gggaagggcagaatgggactccaagcctgcctcctagggctctttgccctcatcctatctggcaaatgcagttacagcccggagcctgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacagagacccatgttgtaaggtccccacatccctaccagcttccacaggccttggccccccatgtggactttgtggggggactgcaccgttttcccccaacatcatccctgaggcaacatcctgagccgcaggtgacagggactgtaggcctgcatctgggggtgaccccctctgtgatccgtaagcgatataacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaacgagtcagccctgccacatgtgcatactgtgagctatggagatgaagaggactccctcagcagcgcctacatccagcgggtcaacactgagctaatgaaggctgccgctcggggtctcaccctgctcttcgcctcaggtgacagtggggccgggtgttggtctgtctctggaagacaccagttccgccctaccttccctgcctccagcccctatgtcaccacggtgggaggcacatccttcctggaacctttcctcaccacaaatgaaattgttgactatatcagtggtggtggcttcagcaatgtgttcccacggccttcataccaggaggaagctgtaaccaagttcctgagctctagcccccacctgccaccatccagttacttcaatgccagtggccgtgcctacccagatgtggctgcactttccgatggctactgggtggtcagcaacagagtgcccattccatgggtgtctggaacctcggcctctactccagtgtttgggggatcctatccttgatcaatgagcacaggatccttagtggccgcccccctcttggctttctcaacccaaggctctaccagcagcatggggcaggactctttgatgtaacccatggctgccatgagtcctgtctggatgaagaggtggagggccagggtttctgctctggccctggctgggatcctgtaacaggctggggaacacccaacttcccagctttgccgaagactctactcaacccctgaccctttcctatcagggaagggcagaatggcttgtcccccgtcctgaagctggcagttcagtcccttattctgccctattggaagccctgctgaaccctcaactattgactgctgcagacagtttatctccctaaccctgaaatgctgtgagcttgactcccaaccccaccatgctccatcatactcaggtctccctactcctgccttagattcctcaataagacactgtaactagcattttttgaatgcctctccctctgcatctcatctttctcttttcaatcaggcttttccaaagggttgtgtacagactctgtgcactatttcacttcatattcactccccaattcactgcaaggagacctctactgtcacctttactctttcctaccctgacatccagaaacaatggactgcagtgcatacttctcaatctttgctttatggcctttccatcatagttgcccactccctctccttacttagtttccagggcttaacttctctgaccactcttgtcttcctctctcatcaacttctgcttcttcatggaatgctgaccttcattgctccatttgtagatttttcctcttctcagtttactcattgtcccctggaacaaatcactgacatctacaaccactaccatctcactaaataagactttctatccaataatgattgatacctcaaatgtaagatgtgtgatactcaacatttcatcgtccaccttcccaaccccaaacaattccatctcgtttcttcttggtaaatgatgctatgctttttccaaccaagccagaaacctgtgtcgtcttttcaccccaccttcaatcaacaagtcctaatgactgcacctcttaaatatatctttatcagtccacaagtccttccaattatatttcccaagtatatctagaacttatccacttatatccccactgctactaccttagtttagggctatattctcttggaaaaaacagtgtccttacttcctgccaatccccaagtcatcttccagagtaaaatgcaaatcccatcaggccacttggatgaaaacccttcaaggattactggatagaattcaggttttttttttgtttttcccccaatcatagctcacaaaccttccttgctatttgttcttaaataaaaaatcatttttcctcctccctccccaaaccccaaggaactctcactcttgctcaagctgttccgtccccttaccacccctgatacaattgcctggtcaaattccagaattcttgcaagactcagttcagaagtcaccttctctcctgaatgttttgattccctgaggctactttattttggtatggctgaaaaatcctagattttctaaacaaaacctgtttgaatcttggttctgatatggactaggagagagactgggtcaagtaagcttatctccctgaggctgtttcctcgtctgttaagtgtgaatatcaatacctgcctttcataatcaccagggaataaagtggaataatgttgataacagtgcttggcacctggaagtaggtggcagatgttaacgcccttcctcccttgcactgcgccccctgtgcctacctctagcattgtaacgaccacatagtattgaaatggccagtttacttgtctggcttcctttccaagaccgttggtgcctagaggactagaatcgtgtcctatttaactttgtattcccaggtcctagctcaggagttggcaaataagaattaaatgtctgctacactggaaaaaaaaaaaaaaaa

SEQ ID NO: 54 - DNA, 2485 bp, Rattus norvegicus
ccaaaatgggactccaagcccgcttcctaggactccttgctctcgtcatcgccggcaaatgcactcacagccccgagccggaccaacggtggatgctgcctccaggctgggtgtcactgggccgagtggatcccgaggaagagctgagtcttacttttgcgctgaaacagcagaacctggacagactctcggagctggtgcaggctgtgtcggatcccagctctcctcgatatggaaagtacctcaccctagaggatgtagctgagctggtccaaccatcaccactgaccctccgcactgtccaaaagtggctcttggcagctggagcccgggactgccattcagtgaccacccaggactttctgacttgctggctgagtgtccggcaggctgagctgctgcttcctggggctgagtttcatcgctatgtagggggacctgcaaagacccacattataaggtccccacatccctatcagctcccccaggccttggcccctcatgtggaccttgtggcggggctgcaccgtttcccccctttatcatctccgagacaacgtccagagccacaaggagtgggacctgttggcctgcacctgggagtgactccatctgtgcttcgtcagcgatataacctgacagcccgagatgtgggctctggcaccaccaacaatagccaggcctgtgcccagttcctggaacagtacttccataactcggatctgactgagttcatgcgcctgtttggtagcagttttgcacaccaggcctcagtagcaagagtggttggaaagcaggggcgaggccgagcgggcatcgaggccagtctagatgtggaatacctgatgagtgctggtgccaatatctccacttgggtctacagtagccctggccgccatgaggcacaggagcccttcttacaatggctcctgcttcttagcaatgagtcatccttgccgcatgtacatactgtgagttacggagacgatgaagactccctcagcagtgtctacatccagagagtcaacactgagttcatgaaggctgctgctcggggtctcaccctcctttttgcctcaggtgacactggagctgggtgttggtctgtctccggaagacacaagttccgtcctagcttccctgcttctagcccctatgtcactacagtaggaggaacctccttcaagaatcctttccttgtcacaaatgaagtagtcgactatatcagtggtggaggcttcagcaatgttttcccacagccttcataccaggaggaagcagtagcccagttcttgaagtccagctctcatctaccaccatctagttacttcaatgctagtggccgtgcttacccagatgttgccgcgctttctgatggctactgggtggtcagcaacatggtccccattccgtgggtatctggaacctcggcctctactccagtgtttgggggaattttatccttgataaatgagcacagactcctcaatggccgcccccctctaggctttctcaatcccaggctctatcagcagcatggggcaggactctttgatgtaacccatggctgccatgagtcctgtctgaatgaagaagtggagggtcagggtttctgctctggtcctggctgggatcctgtgacaggttggggaacacccaacttcccagccctactgaagactctactcaacccttgacccttccctgccctgacgagaaagcagaactgtttcctgtactacaagggaaggctcagtttctcgttattcctcaatggaagccctgatgaactcttaactgttgcctgctgcagatagcttctccctaaccctcaaatgctgtgagcaggactggactcccaaccctgtgagccatcaaactcaggtctccaaacttctacttcgagatccccaacaagatgctataaccagcattttgtctcaccccaaccccatctctccttcctctttccagcttgagatgtgaaagcagggcgagaagattcagtctgcattactgacaccagcagtcccacctaatgcttaccacctctgcactgaccgtatgctctttctcttccagctgcttcttcgttcactgaaatgagacctttgtgctaatcgtttagtctttcttcccctggcactgaagtgcaatggtctccctgacattctatccagttatacacttctcagtgttttatgggctttcttgtttaagaacaggttggtgatgggttggggatttagctcagtggtagagcgcttgcctaggaagcgcaaggccctgggttcggtccccagctccgaaaaaaagaaccaaaaaaaaaaaaagaacaggttggtgacaatctatataactattctaaggaaagttcaagtttgaaaattttaaatgttttaaatgttcttattggcaaagctcctcaacaaaataaataggtgttacaaattttgcttcaaaaaaaaaaaaaaaaaaaaaaaaa

SEQ ID NO: 55 - DNA, 3509 bp, Macaca fascicularis
agcagatctgcggaagggcagaatgggactccaaggctgcctcctagggctgtttgccctcatcctctctggcaaatgcagttacagcccggagcccgaccagcggagaacgctgcccccaggctgggtgtccttgggccgtgcggaccctgaggaagagctgagtctcacctttgccttgagacagcagaacctggaaagactctcggagctggtgcaggctgtgtcggatcccaactctcctcaatacggaaaatacttgaccctagagaatgtggctgatctggtgaggccatccccactgaccctccacatggtgcaaaaatggctcttggcagccggagcccagaaatgccattctgtgatcacacaggactttctgacttgctggctgagcatccggcaagcagagctgctactccctggggctgagtttcatcactatgtgggaggacctacagagacccatgttgtaaggtccccacgtccctaccagcttccacaggccttggccccccatgtggactttgtggggggactgcaccgttttcccccaacatcatccctgaggcaacgtcctgagccgcaggtgacagggactgtaggcctgcatttgggggtgaccccctctgtgatccgtaagcgatataacttgacctcacaagacgtgggctctggcaccagcaataacagccaagcctgtgcccagttcctggagcagtatttccatgactcagacctggctcagttcatgcgcctcttcggtggcaactttgcacatcaggcatcagtaacccgtgtggttggacaacagggccggggccgggccgggattgaggccagtctagatgtgcagtacctgatgagtgctggcgccaacatctccacctgggtctacagtagccctggccggcatgagggacaggagcccttcctgcagtggctcatgctgctcagtaacgagtcagccctgccacatgtgcatactgtgagctatggagatgaggaggactccctcagcagtgcctacatccagcgggtcaacacggagctcatgaaggctgctgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtagccaagttcctgagctctagcccccaccttccaccatccggttacttcaatgccagtggccgtgcctatccagatgtggctgcactttctgatggctactgggtggtcagcaacagagtgcccattccatgggtgtccggaacctcggcctctactccagtgtttggggggctcctatccttgatcaatgagcacagaatcctcagtggccgcccccctcttggctttctcaacccaaggctctaccagcagcatggggcaggactctttgatgtaacccatggctgccatgcatcctgtctggatgaagaggtggagggccagggtttctgctctggccctggctgggatcctgtaacaggctggggaacacccaacttcccagctttgctgaagactctactcaacccctgacattttcctatcaggagagatggcttgtcccctgccctgaagctggcagttcagtcccttattctgccctattggaagccctgctgaaccctcaactattgactgctgcagacagcttatctgcctaaccctgaaatgctgtgagcttgacttgactcccaaccctaccatgctccattatactcaggtctccctactcctgccttagattcctcaataagatgctataactagcatttttttgaatgcatctccctctgcatctcatctttctcttttcaatcaggcttttccaaagggttgtgtacagactctgcactatttcacttcatattcactgcccaattcactgcaaggagacctctactctcaccttttaccttttcctaccctgacatccagaaacaatggcctgcagtgcatacttctcaatctttgctttatggcctttccatcatagttgcccactccctctccttacttagcttccatggcttaacttctctgaccactcttgtcttcctccctcatcagcttctgctacttcatggaatgctgaccttcattgctccatttgtaggtttttgctcttctcagtttactcattgtcccctggaacaaatcactgacatctacaaccattaccatctcactaaataagactttctatccaatagtgattgatacctcaaatgtaagatgtgtgatattcaacatttcatcttccaccttcccaaacccaaacaattccatctctggtttcttcttggtaaatgatgctatgctttttccaaccaagccagaatctgtgtcatctttccaccgcatcttcaatcaacaagtcctcaatcaacaagtcctaatgactgcacctcgtacctcttaaatatctttatcagtccacaagtccttccaatgatatttcccaagtatatctagaacttatccacttatatccccactgctactacattagtttagggctacattctcttgaaaagtacagcgtccttacttccggccaatccccaaatcatcttccagagtaaaatgcaaatcccatcaggccacttggatgaaaaccctggatagagttcaggctttttattttcttcccccaatcatagctcacaaaccttcgttgctatttgttcctaagtaacaaatcatttttcctcctccctccccaaaccccaaggaactctcactcttgctcaagctgttctgtccccttaataccaccccgatacaaactgcctggttaatttccagaattcttacaagattcagctcagaagtcaccttctctcgtgaatgttttgattccctgaggctacttcattttgctatggctgaaaaatcctagattttctaaacaaaacctgtttgaatcttggttctgatatggactgggagagagactgggtcaagcaagtttatctccctgaggctgtttcctcgtctgttaagtgggattattacctgcctttcataatcaccagggaataaagtggaataatgttgataagagtacttggcacctggaagtaggtggcagatgttaatgcccttcctcccttgcactgcgacccctgtgtctacctctagtattgtaacgaccacatagtattgaaatagccactttacttgtctggcttcctttccaagaccgttggtgcctagaggactagaatcgtgttctatttaactttgtattcccaggtcctagctcaggagttggcaaataagaattaaatatctgctacattgaaaatctcaaaaaaaaaaaaaaaaa

SEQ ID NO: 56 - DNA, 1543 bp, Saimiri boliviensis
  misc_feature (564)..(574): n is a, c, g, or t
cctcatcctctctggcaaatgcagttacagcccggagcctgaccagcaaaggacgctgcccccaggctgggtgtccctgggccgtgcggaccctgaggaagagctgagtctcacctttgccctgagacagcagaacctggaaagactctcggagctggtgcaggctgtgtcagatcccaactctcctcaatacggaaaatacctgaccctagaccatgtggctgatctggtgaggccatcctcactgaccctccacacagtgcaaaaatggctcttggcagctggagcccggaactgccattctgtgatcacacaggactttctgacttgctggctgagcatccgacaagcagagctgctgctccctggggctgagtttcatcactatgtgggaggacctgcagagacccatgttgtaaggtccccaaatccctaccagcttccacaggccttggccccccatgtggactttgtggggggactgcaccgttttcccccaacatcattcctgaggcaacgtcctgagccgcagatggcagggactgtaggtatgcatctgggggnnnnnnnnnnngtgatccgtaagcgatataacttgaccgcacagganntgggctctggcaccagcaacaacagccaagcctgtgcccagttcctggagcagtatttccgtgactcagacctggctgagttcatgcgcctcttcggtggcaactttgcacatcaggcatcagtagcccgtgtggttggacaacagggccggggccgggccgggattgaggccagtctagatgtgcagtacctgatgagtgccggtgccaacatctccacctgggtctacagtagccctggccggcatgagggacaggagcccttcctgcagtggctcatgctgctcagtaacgagtcagccctgccacatgtgcatactgtgagctatggagatgatgaggactccctcagtagcgcctacatccagcgggtcaacactgaactcatgaaggctgccactcgaggtctcaccctgctcttcgcctcaggtgacagtggggctgggtgttggtctgtctctggaagacaccagttccgtccttccttccctgcctccagcccctatgtcaccacagtgggaggcacaagcttccagaatcctttccgcatcacaagtgagattgttgactatatcagtggtggtggcttcagcaatgtgttcccacggccttcataccaggaggaagctgtagccaagttcctgaactctggcccccatctgccaccatccagttacttcaatgccagtggccgtgcctacccagatgtggctgcactttccgatggctactgggtggtcagcaacagtgtgcccattccatgggtgtctggaacctcggcctctactccagtgtttggggggctcctatccttgatcaatgagcacagaatcctcagtggccgcccccctcttggctttctcaatccaaggctctaccagcagcatggggcaggactctttgacgtaacccatggctgccatgagtcctgt

SEQ ID NO: 57 - DNA, 3688 bp, Mus musculus
gactacagatcagcctgaaagccaaaatgggactccaagcccgcctcctagggctccttgctctcgtcatcgccggcaaatgcacttacaaccctgagccggaccagcggtggatgctgcctccaggctgggtgtccctgggccgcgtggatcccgaggaagagctgagtctcacttttgcgctgaaacagcggaacctggaaagactctcggagctggtgcaggctgtgtcggatcctagctctcctcaatatggtgcctatcagttggggcaggaagtgggaggcaggaccaggcaggcactggctggtgcagatcatgtagggccatgtttctggtatgacagacaaatgacaataactgccatattcatacacacacacacacacacacacacacacacacacacacaccaaaatttccacctgagttcttcagtcttaaaactaagaacaaatctaagcctagtagcatgaggtaataatcctagctacttggactaaaacaggacgattttgaatccaaggcatgcctgggcagtgtagcaatattgtcaacattaaaaccaattttttttagaggcctagctagctcattgatagggtgggtgcttacggagcatatgaaaaaccctaggttcaagtgccaggaagatggtgggtgtggggtagctagctgtgtttaagaacacatgattcataatcagatctagattgaaatgttcacttaccctaccagctctcaaatggaggactagcacttagtgtgtgtgcatgatcaagtcatggaccttaccccactctcatagccactgccacaccctcctctgttgctagttcaagtcctaaattgtctttggattaatccactctggccattcctcttgctaccaactcagttgtttattctctttctggattactgcccagtacccggactggtttgaagcttcctctccttcaccccactccccagaacagtcccttctctattctagagtattctctatggtgctcaggctggagtgaatccttcccctgcttaaagtcctgtatggtgaggcaagggtgtgtgtgtgtgtgtgtgtgtgtagaatggaggcgagaggaggggggggcatccttagagcacccaagaagctcctcatatgcaggaaagaatgactcaccgttatgttgtttaaaatcaacaaggatgaagagctgagctaaattagaaggttctgtataactggctccaaaagtttggggaaatctttttgaagataagcttaaaatgggatattgaaagtttactaacgtccttgagtttttgtgaccatggtaacacatgctgtgcatttttcctggccatgacgtttcatgtctctcgagccttttcttccctttgactctcactatgcctagccctacacagtcctcctcgggaagttttgatgtcctctgattgaggtccagacagacaacccaggaaagcctcaaatgaggacgagagcctcttctgaatccctgcaggaaagtacctaaccctggaggatgtagctgagctggttcaaccatcacccctgaccctcctcactgtccaaaagtggctctcagcagctggagcccggaactgcgattcagtgaccacccaggactttctgacttgctggctgagtgtccgacaggctgagctgctgctcccaggagctgagtttcatcgctatgtagggggacctacaaagacccatgttataaggtccccacatccctaccagcttccccaggccttggcccctcatgtggattttgtgggggggctgcaccgtttccccccttcatctccaagacaacgtccagaaccacaacaggtaggaactgttagcctgcacttgggagtgactccgtctgtgctccgtcagcgatacaacctgacagccaaagatgtgggctcaggcaccaccaacaatagccaggcctgtgcccagttcctggaacagtacttccataactcggatctgactgagttcatgcgcctattcggtggcagttttacacaccaggcctcagtagcaaaagttgttggaaagcaagggcgaggccgagctgggatcgaggccagtctagatgtggaatacctgatgagtgctggtgccaatatctccacttgggtctacagtagccctggccgccatgaggcacaggagcccttcttacaatggctcctgcttcttagcaatgagtcatctttgccacatgtacatactgtgagttacggagacgatgaagactccctcagcagcatctacatccagagagtcaacactgagttcatgaaggctgctgctcggggtctcaccctcctttttgcctcaggtgacactggagctgggtgttggtctgtctccggaagacacaagttccgccctagcttccctgcttccagcccctatgttactacagttggaggaacctccttcaagaatcctttcctcatcacagatgaagtagttgactatatcagtggtggaggcttcagcaatgttttcccacggcctccctaccaggaggaagcagtggcccagttcttgaaatccagctctcatctaccaccatccagttacttcaatgctagtggccgtgcctacccagatgttgccgcactatctgatggctactgggtggtcagcaacatggtccccattccatgggtatctggaacctcggcctctactccagtgtttgggggaattttatccttgataaatgagcacagaatcctcaatggccgccctcctcttggctttctcaaccccaggctctatcagcagcatgggacaggactctttgatgtaacccacggctgccatgagtcctgtctgaatgaagaagtggagggtcagggtttctgctctggtcctggctgggatcctgtgacaggttggggaacacccaacttcccagccctactgaagaccctgctcaacccttgaccctttcgtgccatgacgagaaagcagaactgttccctgtactaaaagggaaggctcagtttcttgttattcctcgatagaagccctgctgaactcctgttgcctgctgcagatagcttctccctaaccctcagatgctgtgaacaggactcaactctcaatcctactgtgtgccatcaaactcaggtctccaaacttctacttcaagatcctcaacaagatgctataaccagcatattttgtctcaccccaaccccatctctccttcctctttccagcttgagatgtgaaagcagggcaagaaggttcagtcttccattactgacactagcaggtccacccaacgcttaccacctctgcactgaccgtacactctatttctcttcgggtttgcttttccgttcactgaagtgagacctttgactaatcgttttgtctttcttctctcggcactgaagtacaatggtctccccaatgttttatccagttatacccttttcagtgtttgttttatgggttttcttatttaagaacaggttgtcaaaaaaccattaaaaaaaaaaaagaaaaagaacacattggctgcaatctatttaactatataactattctaaggaaagttaaaaattgaaaacttaaaatgtttgaaatgttctcattggcaaaattcctcaacaaaataaataggtcattacaaattttgctttaaatttc

SEQ ID NO: 58 - DNA, 1692 bp, Homo sapiens
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacacgagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctga

(19) United States
(12) Patent Application Publication
(10) Pub. No.: US 2011/0166074 A1
(43) Pub. Date: Jul. 7, 2011

(54) CLN2 TREATMENT OF ALZHEIMER'S DISEASE

(75) Inventors: Frederick R. Maxfield, Chappaqua, NY (US); Peter Lobel, Highland Park, NJ (US)

(73) Assignee: Cornell Research Foundation, Inc., Ithaca, NY (US)

(21) Appl. No.: 12/446,024

(22) PCT Filed: Oct. 18, 2007
(86) PCT No.: PCT/US2007/081771

§ 371 Date: Apr. 17, 2009

Related U.S. Application Data
(60) Provisional application No. 60/829,906, filed on Oct. 18, 2006.

(51) Int. Cl. A61K 38/00; A61K 31/7088; A61P 25/28

(52) U.S. Cl. 514/17.8; 514/44 R; 514/44 A

(57) ABSTRACT

A method of treating Alzheimer's Disease may include administering to a subject in need of such treatment a CLN2 therapeutic having beta-amyloid degradation activity.

### CROSS-REFERENCE TO RELATED APPLICATION

This application claims the benefit of U.S. Provisional Application No. 60/829,906, filed Oct. 18, 2006, the entire contents of which are hereby incorporated herein by reference.

### STATEMENT REGARDING FEDERALLY SPONSORED RESEARCH OR DEVELOPMENT

Support for research leading to subject matter disclosed in this application was provided in part by the National Institutes of Health Grant Nos. NS 34761 and/or DK27083. Accordingly, the United States Government has certain rights with respect to subject matter of this application.

### SUMMARY

The subject matter herein is broadly directed toward a method for the treatment of a neurological disorder, the method including administering to a subject in need of such treatment a CLN2 therapeutic, which is described below. In some embodiments, the neurological disorder may be Alzheimer's Disease.

### BRIEF DESCRIPTION OF THE DRAWINGS

FIG. 1 depicts the degradation of Cy3-labeled fAβ in microglia treated with 100 nM CLN2.

FIG. 2 depicts a high-throughput summary of CLN2 addback experiments.

FIG. 3 depicts the structure of the AAV_{CU}hCLN2 vector.

### DETAILED DESCRIPTION

The subject matter described herein provides a method of treating a neurological disorder, wherein a subject in need of treatment for such a disorder is administered a CLN2 therapeutic.

### 1. DEFINITIONS

For purposes of the present description, the term “isolated” means at the least removed from a natural cellular location. The CLN2 therapeutic may be purified, so that it comprises at least 50%, preferably at least 75%, and more preferably at least 90% of CLN2 protein (in the case of a nucleic acid, of nucleic acids) in a sample.

A composition comprising “A” (where “A” is a single protein, DNA molecule, vector, recombinant host cell, etc.) is substantially free of “B” (where “B” comprises one or more contaminating proteins, DNA molecules, vectors, etc.) when at least about 75% by weight of the proteins, DNA, vectors (depending on the category of species to which A and B belong) in the composition is “A”. “A” may comprise at least about 90% by weight of the A+B species in the composition, or at least about 99% by weight. A composition, which is substantially free of contamination, may contain only a single molecular weight species having the activity or characteristic of the species of interest.

In a specific embodiment, the term “about” means within about 20%, preferably within about 10%, and more preferably within about 5%, of the value modified.

The term “CLN2” (note absence of italics) is interchangeable with “CLN2 protein”, “CLN2 enzyme”, “CLN2 protease”, “CLN2 pepstatin-insensitive carboxyl protease”, “TPP1”, “TPP1 protein”, TPP1 enzyme”, “TPP1 protease”, and “TPP1 pepstatin-insensitive carboxyl protease”. CLN2 refers to a protein having a proteolytic activity and an amino acid sequence with 95%, 96%, 97%, 98%, 99%, and/or 100% identity to a sequence selected from the group consisting of SEQ ID NO:1 through SEQ ID NO:34. The amino acid sequence of Bos taurus CLN2, and variations thereof, are depicted in SEQ ID NO:1 and SEQ ID NO:2. The amino acid sequence of Canis familiaris CLN2, and variations thereof, are depicted in SEQ ID NO:3 and SEQ ID NO:4. The amino acid sequence of Homo sapiens CLN2, and variations thereof, are depicted in SEQ ID NO:5 through SEQ ID NO:11. The amino acid sequence of Macaca fascicularis CLN2, and variations thereof, are depicted in SEQ ID NO:12, SEQ ID NO:14, SEQ ID NO:29, and SEQ ID NO:30. The amino acid sequence of Mus musculus CLN2, and variations thereof, are depicted in SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:33, and SEQ ID NO:34. The amino acid sequence of Pan troglodytes CLN2, and variations thereof, are depicted in SEQ ID NO:23 and SEQ ID NO:24. The amino acid sequence of Pongo pygmaeus CLN2, and variations thereof, are depicted in SEQ ID NO:25 and SEQ ID NO:26. The amino acid sequence of Rattus norvegicus CLN2, and variations thereof, are depicted in SEQ ID NO:27 and SEQ ID NO:28. The amino acid sequence of Saimiri boliviensis CLN2, and variations thereof, are depicted in SEQ ID NO:31 and SEQ ID NO:32. The proteolytic activity may be a beta-amyloid degradation activity.

The term “CLN2” (note presence of italics) is used in reference to a gene, an mRNA, or a cDNA encoding the CLN2 protease. CLN2 has a nucleic acid sequence selected from the group consisting of SEQ ID NO:35-58. A sequence encoding Bos taurus CLN2 is depicted in SEQ ID NO:35. A sequence encoding Canis familiaris CLN2 is depicted in SEQ ID NO:36. Sequences encoding human CLN2 and variations thereof are depicted in SEQ ID NO:37, SEQ ID NO:38, SEQ ID NO:39, SEQ ID NO:40, SEQ ID NO:41, SEQ ID NO:42, and SEQ ID NO:58. Sequences encoding Macaca fascicularis CLN2 and variations thereof are depicted in SEQ ID NO:43, SEQ ID NO:44, and SEQ ID NO:55. Sequences encoding Mus musculus CLN2 and variations thereof are depicted in SEQ ID NO:45, SEQ ID NO:46, SEQ ID NO:47, SEQ ID NO:48, SEQ ID NO:49, SEQ ID NO:50, SEQ ID NO:51, and SEQ ID NO:57. A sequence encoding Pan troglodytes CLN2 is depicted in SEQ ID NO:52. A sequence encoding Pongo pygmaeus CLN2 is depicted in SEQ ID NO:53. A sequence encoding Rattus norvegicus CLN2 is depicted in SEQ ID NO:54. A sequence encoding Saimiri boliviensis CLN2 is depicted in SEQ ID NO:56.

The term “CLN2 therapeutic” is used in reference to a composition including CLN2 or the CLN2 gene or cDNA, or biologically active fragments or portions thereof. The CLN2 therapeutic may be provided in a wide variety of forms, including a polypeptide, a nucleic acid, a vector, a virus, and/or a replication-deficient virus. The CLN therapeutic may also be provided as a cell that includes a CLN2 polypeptide or CLN2 gene or cDNA, such as a cell that has been transfected with a vector encoding a CLN2 gene or cDNA or that has been transgenically modified to carry in its genomic DNA a transgene encoding a CLN2 gene or cDNA. The CLN2 therapeutic may be administered to a subject in need of treatment for a neurological disorder. In one embodiment the neurological disorder is Alzheimer's disease.

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication, i.e., capable of replication under its own control.

A cell has been “transfected” by exogenous or heterologous DNA when such DNA has been introduced inside the cell. A cell has been “transformed” by exogenous or heterologous DNA when the transfected DNA expresses mRNA, which may be translated into a protein. Usually, expression of such a protein effects a phenotypic or functional change in the cell. However, the protein may be expressed without significantly affecting the cell, e.g., in the instance of fermentation of transformed cells for production of a recombinant polypeptide. The transforming DNA may be integrated (covalently linked) into chromosomal DNA making up the genome of the cell.

“Heterologous” DNA refers to DNA not naturally located in the cell, or in a chromosomal site of the cell. The heterologous DNA may include a gene foreign to the cell.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA, and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., infra). The conditions of temperature and ionic strength determine the “stringency” of the hybridization. For preliminary screening for homologous nucleic acids, low stringency hybridization conditions, corresponding to a Tₘ of 55° C. can be used, e.g., 5×SSC, 0.1% SDS, 0.25% milk, and no formamide; or 30% formamide, 5×SSC, 0.5% SDS. Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., infra, 9.50-0.51). For hybridization with shorter nucleic acids, i.e., oligonucleotides, the position of mismatches becomes more important, and the length of the oligonucleotide determines its specificity (see Sambrook et al., infra, 11.7-11.8). A minimum length for a hybridizable nucleic acid may be at least about 10 nucleotides; but may also be at least about 15 nucleotides or at least about 20 nucleotides.

In one embodiment, the term “standard hybridization conditions” refers to a Tₘ of 55° C. and utilizes conditions as set forth above. In another embodiment, the Tₘ is 60° C.; in still another embodiment, the Tₘ is 65° C.

As used herein, the term “oligonucleotide” refers to a nucleic acid, generally of at least 18 nucleotides, that is hybridizable to a genomic DNA molecule, a cDNA molecule, or an mRNA molecule encoding CLN2 Oligonucleotides can be labeled, e.g., with ³²P-nucleotides or nucleotides to which a label, such as biotin, has been covalently conjugated (see the discussion, supra, with respect to labeling polypeptides). In one embodiment, a labeled oligonucleotide can be used as a probe to detect the presence of a nucleic acid encoding CLN2. In another embodiment, oligonucleotides (one or both of which may be labeled) can be used as PCR primers, either for cloning full length or a fragment of CLN2, or to detect the presence of nucleic acids encoding CLN2. In a further embodiment, an oligonucleotide can form a triple helix with a CLN2 DNA molecule. Oligonucleotides may be prepared synthetically on a nucleic acid synthesizer. Accordingly, oligonucleotides can be prepared with non-naturally occurring phosphoester analog bonds, such as thioester bonds, etc.

“Homologous recombination” refers to the insertion of a foreign DNA sequence of a vector in a chromosome. The vector may target a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

Transcriptional and translational “control sequences” are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A coding sequence is “under the control of”, “operably associated with”, or “operatively associated with” transcriptional and translational (i.e. expression) control sequences in a cell when RNA polymerase transcribes the coding sequence into mRNA, which is then trans-RNA spliced and translated into the protein encoded by the coding sequence.

A “signal sequence” is included at the beginning of the coding sequence of a protein to be expressed on the surface of a cell. This sequence encodes a signal peptide, N-terminal to the mature polypeptide, that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

### 2. THE CLN2 THERAPEUTIC

Alzheimer's amyloid-beta fibrils (fAβ), like other extracellular debris present in brain tissue, are normally ingested by microglial cells. Microglial cells are the primary phagocytic cells within the central nervous system (“CNS”). Ingestion occurs by means of an endocytic process, and the endocytosed fAβ is delivered to digestive organelles called late endosomes or lysosomes (LE/LY). Most material ingested in this manner is degraded in LE/LY, but the internalized fAβ are not degraded efficiently after internalization by microglia in cell culture. Addition of lysosomal enzymes to microglial cells in cell culture increases their ability to digest fAβ by approximately 40-50%. Additionally, administration of a single lysosomal enzyme called CLN2 allows microglia to digest fAβ to approximately 20 to 60% of completion. Thus, administration of CLN2 either directly (i.e. administration of protein), via gene therapy, and/or via cell therapy, such that an elevated level of CLN2 within cells is brought about, is a novel way to treat a subject having Alzheimer's Disease in need of such treatment. In one embodiment, the cell in which the CLN2 level becomes elevated is a microglial cell. Different treatment modalities may be combined (i.e., both protein and gene therapy, both protein and cell therapy, both cell and gene therapy, or protein, gene, and cell therapy).

The neurological disorder may also be any disorder that exhibits pathology involving the accumulation of abnormal protein deposits in the brain. Such disorders include but are not limited to Alzheimer's disease, Parkinson's Disease, Huntington's Disease, any one of several forms of Transmissible Spongiform Encephalopathy (e.g. Classic Creutzfeldt-Jakob Disease, New Variant Creutzfeldt-Jakob Disease, Bovine Spongiform Encephalopathy, Gerstmann-Straussler-Scheinker Syndrome, Fatal Insomnia, Kuru), Dementia with Lewy Bodies, Frontotemporal Lobar Degeneration, Pick's Disease, Batten's Disease, Neural Ceroid Lipofuscinosis (NCL, e.g. Infantile NCL, Late Infantile NCL, Juvenile NCL, Adult NCL), Frontotemporal Dementia, and Semantic Dementia.

In some embodiments, the CLN2 therapeutic may include a polypeptide having a sequence with 95%, 96%, 97%, 98%, 99%, and/or 100% identity to a sequence selected from the group consisting of SEQ ID NO:1 through SEQ ID NO:34. The CLN2 therapeutic may further include a transcytosis peptide: i.e., a peptide capable of being drawn into a cell, transported across its interior, and ejected on the other side, to help the CLN2 therapeutic to gain entry into, or gain passage through, cells in the target tissue such that it may cross the blood-brain barrier. In another embodiment, the therapeutic may include a biologically active fragment or portion of a polypeptide, the polypeptide having a sequence with 95%, 96%, 97%, 98%, 99%, and/or 100% identity to a sequence selected from the group consisting of SEQ ID NO:1 through SEQ ID NO:34, wherein the fragment has proteolytic activity. The proteolytic activity may be fAβ proteolytic activity.

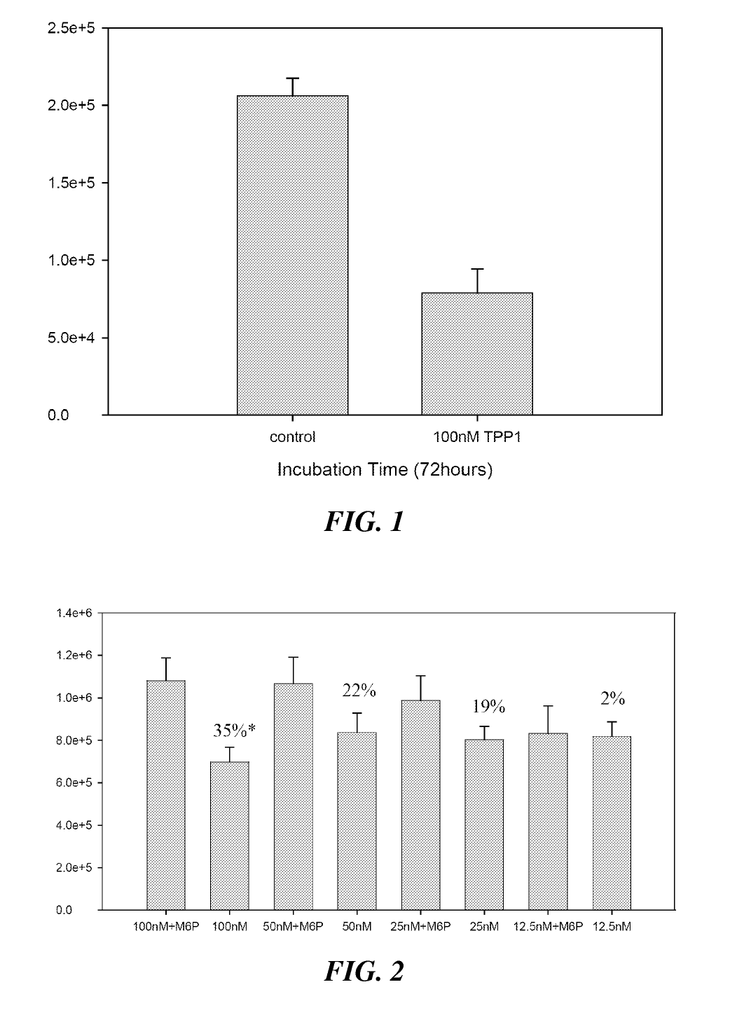

As may be appreciated from comparison of these sequences, the CLN2 sequences and variations thereof are well-conserved across mammalian species (See, for example, the CLN2 sequence alignment in FIG. 1 of Wlodawer et al., “A model of tripeptidyl-peptidase I (CLN2), a ubiquitous and highly conserved member of the sedolisin family of serine-carboxyl peptidases.” BMC Struct Biol. 2003 Nov. 11; 3:8). Certain residues of SEQ ID NO:5 and the other amino acid sequences appear to form part of CLN2's active site, such as residues 272 (Glu), 276 (Asp), 360 (Asp), and 475 (Ser), and certain residues appear to be important in maintaining stability of the enzyme, such as residues 447 (Arg), 451 (Asp), 517 (Asp), and 543 (Asp) (although in mouse the residue numbers are each one less owing to the absence of human residue 179 from the mouse protein). Consequently, recombinant variants of the CLN2 proteins may exclude one or more of these residue from mutation, in order to preserve enzyme function.

In some embodiments, the CLN2 therapeutic may include a chimeric protein. In some embodiments, the chimeric protein consists of maltose binding protein or poly-histidine with CLN2. However, the CLN2 therapeutic may also comprise a chimeric protein comprising a targeting moiety with CLN2. The targeting moiety may be an intracellular targeting moiety.

In some embodiments, the CLN2 therapeutic may include a nucleic acid having at least 95%, 96%, 97%, 98%, 99%, and/or 100% similarity to a nucleotide sequence which, in turn, encodes a polypeptide comprising a sequence selected from the group consisting of SEQ ID NO:1 through SEQ ID NO:34. In some embodiments, the CLN2 therapeutic may include a nucleic acid which itself encodes a protein having at least 95%, 96%, 97%, 98%, 99%, and/or 100% identity to a sequence selected from the group consisting of SEQ ID NO:1 through SEQ ID NO:34. In some embodiments, the CLN2 therapeutic may include a nucleic acid that hybridizes under stringent conditions to the complementary strand of a nucleic acid comprising a sequence selected from the group consisting of SEQ ID NO:35 through SEQ ID NO:58.

In some embodiments, the CLN2 therapeutic may include a nucleic acid which encodes a biologically active fragment or portion of a polypeptide, the polypeptide having a sequence with 95%, 96%, 97%, 98%, 99%, and/or 100% identity to a sequence selected from the group consisting of SEQ ID NO:1 through SEQ ID NO:34, wherein the fragment has proteolytic activity. The proteolytic activity may be fAβ proteolytic activity. In one embodiment, the CLN2 therapeutic may include a nucleic acid which encodes a polypeptide that includes amino acid residues 16-563 of SEQ ID NO:5. In another embodiment, the CLN2 therapeutic may include a nucleic acid which encodes a polypeptide that includes amino acid residues 196-563 of SEQ ID NO:5.

In some embodiments, the CLN2 therapeutic may include a nucleic acid which encodes a chimeric CLN2 protein. In some embodiments, the chimeric protein consists of maltose binding protein or poly-histidine with CLN2. However, the CLN2 therapeutic may also include a nucleic acid which encodes a chimeric protein comprising a targeting moiety with CLN2. The targeting moiety may be an intracellular targeting moiety.

The CLN2 therapeutic may be administered directly, or it may be administered via a gene therapy technique. A gene therapy approach may necessitate the use of a vector, such as a virus, to deliver the appropriate nucleic acid to the target cells. Thus, the CLN2 therapeutic may include a recombinant DNA vector that includes any of the nucleic acids mentioned above. The DNA vector may be an expression vector, wherein DNA encoding CLN2 is operatively associated with an expression control sequence, whereby transformation of a host cell with the expression vector provides for expression of CLN2, or a fragment thereof. The vector may be an expression vector designed to introduce a desired nucleic acid into a target cell, wherein the nucleic acid will be expressed such that a desired protein encoded by the nucleic acid sequence is produced by the cellular transcription and translation machinery. The vector may further comprise a promoter, leading to efficient transcription of the nucleic acid carried on the expression vector. Any type of promoter is acceptable, including but not limited to a constitutive promoter or an inducible promoter.

The subject matter further provides a recombinant virus comprising the DNA expression vector for use as a CLN2 therapeutic. The recombinant virus may be selected from the group consisting of a retrovirus, herpes simplex virus (HSV), papillomavirus, Epstein Barr virus (EBV), adenovirus, and adeno-associated virus (AAV).

The cells or cell-types targeted by the CLN2 therapeutic may include any cell normally found within the central nervous system (e.g. neurons, microglia, and glia) wherein abnormal protein deposits associated with a neurological disorder may accumulate, or whereto such abnormal protein deposits may be delivered. In one embodiment, the target cell may be a microglial cell. As used herein, “glia” may refer to astrocytes, oligodendrocytes, ependymal cells, or radial glia, among others.

The CLN2 therapeutic may itself comprise a cell as just described. Alternatively or additionally, the CLN2 therapeutic may comprise a cell engineered to produce CLN2 by a process utilizing of any of the nucleic acids or vectors described above. In a particular embodiment the cell may be a stem cell, such as a multipotent self-renewing central nervous system neural stem cell. In this case, the multipotent self-renewing central nervous system neural stem cell population may be obtained from a human (e.g., HuCNS-SC). The cells of the multipotent CNS neural stem cell population can be proliferated in a suspension culture or in an adherent culture prior to administration to the mammal or prior to the manufacture of the medicament.

In some embodiments, the CLN2 therapeutic may include a chimeric or fusion protein formed in part from CLN2 or a fragment thereof, thus termed a “CLN2 fusion protein,” or a nucleic acid encoding the CLN2 fusion protein. A CLN2 fusion protein may include at least one functionally active portion of a non-CLN2 protein (termed herein the “fusion partner”) joined via one or more peptide bonds to one or more functionally active portions of a CLN2 polypeptide. The non-CLN2 sequence(s) can be amino- or carboxyl-terminal relative (or both) to the CLN2 sequence(s). In some embodiments, CLN2 is expressed as a fusion protein in which the fusion partner is maltose binding protein or poly-histidine, said fusion parters facilitating purification of the CLN2 fusion protein using affinity chromatography. Affinity chromatography may be performed using a nickel substrate to obtain substantially pure CLN2 fusion protein in which poly-histidine is the fusion parter. Affinity chromatogrpahy may also be performed using a substrate made from starch, cellulose, amylose, or any other complex carbohydrate, to obtain substantially pure CLN2 fusion protein in which maltose binding protein is the fusion parter. In some embodiments, CLN2 fusion proteins may be made using a wide variety of other fusion partners, including marker proteins such as lacZ, signal peptides for extracellular or periplasmic expression, and different lysosomal localization peptides, to mention but a few possibilities. CLN2, or a polypeptide fragment domain thereof, may also be joined with a non-CLN2 protein to create a hybrid fusion protein having different target specificity, particularly targeting for intracellular translocation, catalytic activity, or other combinations of properties from the CLN2 or fragment thereof with the fusion partner. A recombinant DNA molecule encoding such a fusion protein may include a sequence encoding at least one functionally active portion of a non-CLN2 protein joined in-frame contiguously or non-contiguously to at least one CLN2 coding sequence or portion thereof. It may encode a cleavage site for a specific protease, e.g., thrombin or Factor Xa, such as at the CLN2-non-CLN2 juncture. In some embodiments, the CLN2 fusion protein is expressed in Escherichia coli.

### 3. GENES ENCODING CLN2 PROTEASE

The subject matter contemplates a method of treating a neurological disorder comprising administering to a subject in need of such treatment a CLN2 therapeutic. The CLN2 therapeutic may comprise a gene encoding a CLN2 protein, including a full length, or naturally occurring form of CLN2, and any antigenic fragments thereof from any animal, particularly mammalian or avian, and more particularly human, source. The CLN2 therapeutic may also comprise a gene encoding a biologically active fragment or portion of the CLN2 protein, wherein the fragment has proteolytic activity. The proteolytic activity may be fAβ proteolytic activity. As used herein, the term “gene” refers to an assembly of nucleotides that encode a polypeptide, and includes cDNA and genomic DNA nucleic acids.

A method of treating a neurological disorder may include administering two CLN2 therapeutics, in which one therapeutic includes a nucleic acid encoding CLN2 (or a protein having a degree of identity to a CLN2 sequence, as described elsewhere herein), and the other including a CLN2 protein (or a protein having a degree of identity to a CLN2 sequence, as described elsewhere herein). In some embodiments, the protein therapeutic may be administered first, followed by the nucleic acid therapeutic.

In accordance with the subject matter there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

The CLN2 therapeutic may be comprised of a gene encoding CLN2, whether genomic DNA or cDNA, which can be isolated from any source, including from a human cDNA or genomic library. Methods for obtaining the CLN2 gene are well known in the art, as described above (see, e.g., Sambrook et al., 1989, supra).

Accordingly, any animal cell potentially can serve as the nucleic acid source for the molecular cloning of a CLN2 gene for use as a CLN2 therapeutic. The DNA may be obtained by standard procedures known in the art from cloned DNA (e.g., a DNA “library”), and may be obtained from a cDNA library prepared from tissues with high level expression of the protein, by chemical synthesis, by cDNA cloning, or by the cloning of genomic DNA, or fragments thereof, purified from the desired cell (See, for example, Sambrook et al., 1989, supra; Glover, D. M. (ed.), 1985, DNA Cloning: A Practical Approach, MRL Press, Ltd., Oxford, U.K. Vol. I, II). Clones derived from genomic DNA may contain regulatory and intron DNA regions in addition to coding regions; clones derived from cDNA will not contain intron sequences. Whatever the source, the gene should be molecularly cloned into a suitable vector for propagation of the gene.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the desired CLN2 gene may be accomplished in a number of ways. For example, if an amount of a portion of a CLN2 gene or its specific RNA, or a fragment thereof, is available and can be purified and labeled, the generated DNA fragments may be screened by nucleic acid hybridization to the labeled probe (Benton and Davis, 1977, Science 196:180; Grunstein and Hogness, 1975, Proc. Natl. Acad. Sci. U.S.A. 72:3961). For example, a set of oligonucleotides corresponding to the cDNA for the CLN2 protein can be prepared and used as probes for DNA encoding CLN2, or as primers for cDNA or mRNA (e.g., in combination with a poly-T primer for RT-PCR). A fragment may be selected that is highly unique to CLN2, the sequence of which is selected from the group consisting of SEQ ID NO:35 through SEQ ID NO:58. Those DNA fragments with substantial sequence similarity to the probe will hybridize. As noted above, the greater the degree of sequence similarity, the more stringent are the hybridization conditions that can be used. In one embodiment, low stringency hybridization conditions (50° C., 50% formamide, 5×SSC, 5×Denhardts solution) can be used to identify a homologous CLN2 gene, preferably a human CLN2 gene, using a murine CLN2 cDNA probe.

Further selection can be carried out on the basis of the properties of the gene, e.g., if the gene encodes a protein product having the isoelectric, electrophoretic, amino acid composition, uniquely characteristic set of structural domains, or partial amino acid sequence of CLN2 protein as disclosed herein. Thus, the presence of the gene may be detected by assays based on the physical, chemical, or immunological properties of its expressed product. For example, the rabbit polyclonal antibody to murine or human CLN2 may be used to confirm expression of CLN2. In another aspect, a protein that has an apparent molecular weight of 46 kDa, and which is biochemically determined to have tripeptidyl-peptidase I activity, is a good candidate for CLN2.

The present invention also relates to cloning vectors containing genes encoding CLN2, active fragments thereof, analogs, and derivatives of CLN2, used to comprise a CLN2 therapeutic, that have the same or homologous functional activity as CLN2, and homologs thereof from other species. The production and use of derivatives and analogs related to CLN2, used to comprise a CLN2 therapeutic, are within the scope of the present invention. For example, a fragment corresponding to the catalytic domain exhibits enzymatic activity. In a specific embodiment, the derivative or analog is functionally active, i.e., capable of exhibiting one or more functional activities associated with a full-length, wild-type CLN2 of the invention.

CLN2 derivatives can be made by altering encoding nucleic acid sequences by substitutions, additions or deletions that provide for functionally equivalent molecules. Derivatives may be made that have enhanced or increased functional activity relative to native CLN2.

Due to the degeneracy of nucleotide coding sequences, other DNA sequences which encode substantially the same amino acid sequence as a CLN2 gene may be used in the practice of the present invention. These include but are not limited to allelic genes, homologous genes from other species, and nucleotide sequences comprising all or portions of CLN2 genes which are altered by the substitution of different codons that encode the same amino acid residue within the sequence, thus producing a silent change. Likewise, the CLN2 derivatives, used to comprise a CLN2 therapeutic, include but are not limited to, those containing, as a primary amino acid sequence, all or part of the amino acid sequence of a CLN2 protein including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point.

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free —NH₂ can be maintained.

Substitutions of Glu for Asp and visa versa, or “switching” acid amino acid residues with other residues, while retaining the total number of acidic residues in the acidic domain, are expected to retain the functional activity of that domain.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

The genes encoding CLN2 derivatives and analogs, used to comprise a CLN2 therapeutic, can be produced by various methods known in the art. The manipulations which result in their production can occur at the gene or protein level. For example, the cloned CLN2 gene sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of the gene encoding a derivative or analog of CLN2, care should be taken to ensure that the modified gene remains within the same translational reading frame as the CLN2 gene, uninterrupted by translational stop signals, in the gene region where the desired activity is encoded.

Additionally, the CLN2-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Such mutations may enhance the functional activity of the mutated CLN2 gene product. Any technique for mutagenesis known in the art can be used, including but not limited to, in vitro site-directed mutagenesis (Hutchinson, C., et al., 1978, J. Biol. Chem. 253:6551; Zoller and Smith, 1984, DNA 3:479-488; Oliphant et al., 1986, Gene 44:177; Hutchinson et al., 1986, Proc. Natl. Acad. Sci. U.S.A. 83:710), use of TAB linkers (Pharmacia), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pMal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. The cloned gene may be contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences form the yeast 2μ plasmid.

### 4. EXPRESSION OF CLN2 PROTEINS

The nucleotide sequence coding for CLN2, or antigenic fragment, derivative or analog thereof, or a functionally active derivative, including a chimeric protein, thereof, used to comprise a CLN2 therapeutic, can be inserted into an appropriate expression vector, i.e., a vector which contains the necessary elements for the transcription and translation of the inserted protein-coding sequence. Such elements are termed herein a “promoter.” Thus, the nucleic acid encoding CLN2, used to comprise a CLN2 therapeutic, is operably associated with a promoter in an expression vector of the invention. Both cDNA and genomic sequences can be cloned and expressed under control of such regulatory sequences. An expression vector may also include a replication origin, unless the vector is intended for homologous recombination.

The necessary transcriptional and translational signals can be provided on a recombinant expression vector, or they may be supplied by the native gene encoding CLN2 and/or its flanking regions.

As pointed out above, potential chimeric partners for CLN2, used to comprise a CLN2 therapeutic, include substitute catalytic domains, or a different nuclear targeting domain.

Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant CLN2 protein, used to comprise a CLN2 therapeutic, or functional fragment, derivative, chimeric construct, or analog thereof, may be expressed chromosomally, after integration of the coding sequence by recombination. In this regard, any of a number of amplification systems may be used to achieve high levels of stable gene expression (See Sambrook et al., 1989, supra).

The cell into which the recombinant vector comprising the nucleic acid encoding the CLN2 therapeutic is cultured in an appropriate cell culture medium under conditions that provide for expression of CLN2 by the cell.

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

Expression of CLN2 protein, used to comprise a CLN2 therapeutic, may be controlled by any promoter/enhancer element known in the art, but these regulatory elements must be functional in the host selected for expression. Promoters which may be used to control CLN2 gene expression include, but are not limited to, the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94), myelin basic protein gene control region which is active in oligodendrocyte cells in the brain (Readhead et al., 1987, Cell 48:703-712), myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

Expression vectors containing a nucleic acid encoding CLN2, used to comprise a CLN2 therapeutic, can be identified by four general approaches: (a) PCR amplification of the desired plasmid DNA or specific mRNA, (b) nucleic acid hybridization, (c) presence or absence of selection marker gene functions, (d) analysis with appropriate restriction endonucleases, and (e) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if the nucleic acid encoding CLN2 is inserted within the “selection marker” gene sequence of the vector, recombinants containing the CLN2 insert can be identified by the absence of reading frames), pAc360 (BamHI cloning site 36 base pairs downstream of a polyhedrin initiation codon: Invitrogen (195)), and pBlueBacHisA, B, C (three different reading frames, with BamH1, BglII, PstI, NcoI, and HindIII cloning site, an N-terminal peptide for ProBond purification, and blue/white recombinant screening of plaques: Invitrogen (220)) can be used.

Mammalian expression vectors that may be used include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR; see Kaufman, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and MI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, XhoI, NotI, NheI, HindIII, NheI, PvuII, and KpnI cloning site; constitutive RSV-LTR promoter, hygromycin selectable marker; Invitrogen), pCEP4 (BamH1, SfiI, XhoI, NotI, NheI, HindIII, NheI, PvuII, and KpnI cloning site; constitutive hCMV immediate early gene, hygromycin selectable marker; Invitrogen), pMEP4 (KpnI, PvuI, NheI, HindIII, NotI, XhoI, SfiI, BamH1 cloning site; inducible metallothionein IIa gene promoter, hygromycin selectable marker; Invitrogen), pREP8 (BamH1, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman. 1991, supra) for use according to the invention include but are not limited to pSC11 (SmaI cloning site; TK- and β-gal selection), pMJ601 (SalI, SmaI, AflI, NarI, BspMII, BamHI, ApaI, NheI, SacII, KpnI, and HindIII cloning site; TK- and β-gal selection), and pTKgptF1S (EcoRI, PstI, SalI, AccI, HindIII, SbaI, BamHI, and Hpa cloning site; TK or XPRT selection).

Yeast expression systems may also be utilized to express CLN2 protein for use in comprising a CLN2 therapeutic. For example, the non-fusion pYES2 vector (XbaI, SphI, ShoI, NotI, GstXI, EcoRI, BstXI, BamH1, SacI, KpnI, and HindIII cloning site; Invitrogen) or the fusion pYESHisA, B, C (XbaI, SphI, ShoI, NotI, BstXI, EcoRI, BamH1, SacI, KpnI, and HindIII cloning site; N-terminal peptide purified with ProBond resin and cleaved with enterokinase; Invitrogen), to mention just two, can be employed.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda); and plasmid and cosmid DNA vectors, to name but a few.

In addition, a host cell strain may be chosen which modulates the expression of the inserted sequences, or modifies and processes the gene product in the specific fashion desired. Different host cells have characteristic and specific mechanisms for the translational and post-translational processing and modification (e.g., glycosylation, cleavage [e.g., of signal sequence]) of proteins. Appropriate cell lines or host systems can be chosen to ensure the desired modification and processing of the foreign protein expressed. For example, expression in a bacterial system can be used to produce an nonglycosylated core protein product. Expression in yeast can produce a glycosylated product. Expression in eukaryotic cells can increase the likelihood of “native” folding of a heterologous protein. Moreover, expression in mammalian cells can provide a tool for reconstituting, or constituting, CLN2 activity for use in comprising a CLN2 therapeutic. Furthermore, different vector/host expression systems may affect processing reactions, such as proteolytic cleavages, to a different extent.

Vectors are introduced into the desired host cells by methods known in the art, e.g., transfection, electroporation, microinjection, transduction, cell fusion, DEAE dextran, calcium phosphate precipitation, lipofection (fusion of liposomes with cell membranes), use of a gene gun (biolistics), or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

### 5. THERAPEUTIC ASPECTS OF CLN2

Based on the data depicted in the drawings, infra, particularly the observation that treatment of murine microglia cells with CLN2 promotes degradation of intracellular Cy3-labelled amyloid-beta fibrils (Cy3-fAβ), CLN2 may be employed in a particular embodiment as a therapeutic to attenuate the accumulation of abnormal protein deposits associated with Alzheimer's disease. Thus, CLN2, or an expression vector encoding CLN2, can be administered to a subject in need of treatment for Alzheimer's disease in order to enhance the degradation of intracellular amyloid-beta fibrils (fAβ) associated with the disease.

CLN2 may also be employed as a therapeutic to attenuate the accumulation of abnormal protein deposits associated with a variety of additional neurodegenerative diseases, each of which exhibits pathology involving the accumulation of abnormal protein deposits in the brain. These diseases include but are not limited to:

Parkinson's disease, characterized by the abnormal accumulation of the protein alpha-synuclein.

Huntington's disease, a disorder that involves the continued aggregation of an abnormal Huntington protein.

Any one of several forms of Transmissible Spongiform Encephalopathy (TSE), including but not limited to Classic Creutzfeldt-Jakob Disease, New Variant Creutzfeldt-Jakob Disease, Bovine Spongiform Encephalopathy, Gerstmann-Straussler-Scheinker Syndrome, Fatal Insomnia, and Kuru, each of which is characterized by the abnormal accumulation of a prion protein.

Dementia with Lewy Bodies, a disorder that exhibits clinical overlap between Alzheimer's Disease and Parkinson's Disease and is characterized by the development of abnormal proteinaceous inclusions containing the alpha-synuclein protein.

Frontotemporal Lobar Degeneration (FTLD), which in certain manifestations is characterized either by the presence of inclusions of the tau protein with or without Pick bodies, the presence of ubiquitin-positive tau-negative proteinaceous inclusions, or both.

Pick's Disease, a specific manifestation of FTLD in which tau inclusions with Pick bodies are present.

Batten's disease, which is characterized by the abnormal accumulation of lipopigment deposits, including but not limited to lipofuscin.

Neural Ceroid Lipofuscinosis (NCL), a group of neurodegenerative disorders that result from excessive accumulation of lipopigments, such as lipofuscin, and includes Infantile NCL, Late Infantile NCL, Juvenile NCL, and Adult NCL.

Frontotemporal Dementia (FTD), which may involve different pathologies including Pick's disease, other tau-positive pathology, and ubiquitin-positive tau-negative proteinaceous inclusions.

Semantic Dementia (SD), which is most commonly characterized by the presence of ubiquitin-positive tau-negative proteinaceous inclusions, and less commonly by other pathologies including Pick's disease and other tau-positive pathology.

Various mechanisms are available for delivering CLN2 into cells. CLN2 may be delivered by direct administration of a construct (chimeric or via chemical derivitization or crosslinking) of CLN2 with a targeting molecule (e.g., transferrin, a hormone, a growth factor, a target cell-specific antibody, or a chemical functional group) to a subject in need of treatment. In some embodiments, the targeting molecule may target CLN2 for microglial uptake (e.g. an IgG domain that targets microglial Fc receptors, maleylation of CLN2 for targeting to the microglial scavenger receptor A). CLN2 may also be delivered by gene therapy approaches to increase expression of CLN2 in proliferating cells in situ, or by cell therapy approaches to introduce cells that produce CLN2 into the affected area(s) of the brain.

Cells targeted by the CLN2 therapeutic may include any cell found within the central nervous system, wherein abnormal protein deposits associated with a neurological disorder may accumulate, or whereto such abnormal protein deposits may be delivered. Such cell types include but are not limited to neurons, microglia, and glia. Acceptable cell targets also include granulocytes (e.g. neutrophil granulocytes, eosinophil granulocytes, basophil granulocytes), mastocytes, monocytes (e.g. histiocytes, macrophages, dendritic cells, Langerhans cells, microglia, Kupffer cells, osteoclasts), megakaryoblasts, megakaryocytes, platelets, or any cell derived from myeloid progenitor cells and having endocytic activity. In one embodiment, the targeted cell is a microglial cell.

The term “endocytic activity” refers to any process wherein particles are enveloped by the cell membrane of a nearby cell and internalized to form an endosome. The resulting endosome may be merged with late endosomes or lysosomes (LE/LY) containing digestive enzymes, including but not limited to enzymes exhibiting proteolytic activity. In one embodiment, the enzyme may be CLN2.

A subject in whom administration of CLN2 is an effective therapeutic regimen for a neurological disorder may be a human, but can be any animal. Thus, as can be readily appreciated by one of ordinary skill in the art, the methods and pharmaceutical compositions of the present invention are particularly suited to administration to any animal, particularly a mammal, including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

A CLN2 therapeutic for treatment of a neurological disorder may be provided in a pharmaceutically acceptable carrier or excipient. The phrase “pharmaceutically acceptable” refers to molecular entities and compositions that are physiologically tolerable and do not typically produce an allergic or similar untoward reaction, such as gastric upset, dizziness and the like, when administered to a human. As used herein, the term “pharmaceutically acceptable” may mean approved by a regulatory agency of the Federal or a state government or listed in the U.S. Pharmacopeia or other generally recognized pharmacopeia for use in animals, and more particularly in humans, although a pharmaceutically acceptable carrier of the invention may share the attributes of such an approved carrier without itself having been approved. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions may be employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition in the host. Where amelioration of a neurological disorder is sought, a therapeutically effective amount of a pharmaceutical composition, used to comprise a CLN2 therapeutic, will restore or enhance tripeptidyl-peptidase I activity to levels that ameliorate the neurological disorder. A therapeutically effective amount and treatment regimen can be developed for an individual by an ordinary skilled physician, taking into account the age, sex, size, and physical well being, of the patient; the course and extent of the disease or disorder; previous, concurrent, or subsequent treatment regimens and the potential for drug interactions; all of which parameters are routinely considered by a physician in prescribing administration of a pharmaceutical agent.

The subject matter described herein provides for conjugating targeting molecules to CLN2, DNA vectors (including viruses) encoding CLN2, and carriers (i.e., liposomes) for targeting to a desired cell or tissue, e.g., a tumor. “Targeting molecule” as used herein shall mean a molecule which, when administered in vivo, localizes to desired location(s).

In various embodiments, the targeting molecule can be a peptide or protein, antibody, lectin, carbohydrate, steroid, or a chemical functional group. In one embodiment, the targeting molecule is a carbohydrate, protein, or peptide ligand of an internalized receptor on the target cell. In another embodiment, the targeting molecule is mannose-6-phosphate, a carbohydrate derivative that may serve as a ligand of an internalized receptor on the target cell. In still another embodiment, one or more targeting molecules may be combined to increase the efficiency by which the CLN2 therapeutic is taken up by a target cell or a group of target cells.

In another embodiment, the targeting molecule is a peptide comprising the well known RGD sequence, or variants thereof that bind RGD receptors on the surface of cells such as cancer cells, e.g., human ova that have receptors that recognize the RGD sequence. Other ligands include, but are not limited to, transferrin, insulin, amylin, and the like. Receptor internalization is preferred to facilitate intracellular delivery of CLN2 protein.

In still another embodiment, the targeting molecule is an antibody. The targeting molecule may be a monoclonal antibody. In one embodiment, to facilitate cros slinking the antibody can be reduced to two heavy and light chain heterodimers, or the F(ab′)₂ fragment can be reduced, and crosslinked to the CLN2 via the reduced sulfhydryl.

Antibodies for use as targeting molecule are specific for cell surface antigen. In one embodiment, the antigen is a receptor. For example, an antibody specific for a receptor on cancer cells, such as melanoma cells, can be used. Alternatively, an antibody specific for a receptor on microglial cells may be used.

This invention further provides for the use of other targeting molecules, such as lectins, carbohydrates, proteins and steroids.

### 6 ADMINISTRATION OF TARGETED CLN2

According to the subject matter, a composition comprising delivery of the CLN2 therapeutic may be introduced parenterally, transmucosally, e.g., orally, nasally, or rectally, or transdermally Administration may be parenteral, e.g., via intravenous injection, and also including, but is not limited to, intra-arteriole, intramuscular, intradermal, subcutaneous, intraperitoneal, intraventricular, and intracranial administration.

In another embodiment, the therapeutic compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid). To reduce its systemic side effects and increase cellular penetration, this may be a preferred method for introducing CLN2 for the purposes of treating a neurological disorder.

In yet another embodiment, the therapeutic compound can be delivered in a controlled release system. For example, the CLN2 therapeutic may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, thus requiring only a fraction of the systemic dose (see, e.g., Goodson, in Medical Applications of Controlled Release, supra, vol. 2, pp. 115-138 (1984)). A controlled release device may be introduced into a subject in proximity of the site of tissue affected by the neurological disorder.

Other controlled release systems are discussed in the review by Langer (Science 249:1527-1533 (1990)).

### 7. GENE THERAPY

In one embodiment, a gene encoding a CLN2 protein or polypeptide domain fragment thereof, used to comprise a CLN2 therapeutic, is introduced in vivo or ex vivo in a nucleic acid vector. Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques 7:980-990 (1992)). DNA vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Thus, tumor tissue can be specifically targeted. Examples of particular vectors include, but are not limited to, a defective herpes virus 1 (HSV1) vector (Kaplitt et al., 1991, Molec. Cell. Neurosci. 2:320-330), an attenuated adenovirus vector, such as the vector described by Stratford-Perricaudet et al. (1992, J. Clin. Invest. 90:626-630), and a defective adeno-associated virus vector (Samulski et al., 1987, J. Virol. 61:3096-3101; Samulski et al., 1989, J. Virol. 63:3822-3828).

For in vivo administration, an appropriate immunosuppressive treatment may be employed in conjunction with the viral vector, e.g., adenovirus vector, to avoid immuno-deactivation of the viral vector and transfected cells. For example, immunosuppressive cytokines, such as interleukin-12 (IL-12), interferon-γ (IFN-γ), or anti-CD4 antibody, can be administered to block humoral or cellular immune responses to the viral vectors (see, e.g., Wilson, Nature Medicine (1995)). In addition, it is advantageous to employ a viral vector that is engineered to express a minimal number of antigens.

In another embodiment the gene can be introduced in a retroviral vector, e.g., as described in Anderson et al., U.S. Pat. No. 5,399,346; Mann et al., 1983, Cell 33:153; Temin et al., U.S. Pat. No. 4,650,764; Temin et al., U.S. Pat. No. 4,980,289; Markowitz et al., 1988, J. Virol. 62:1120; Temin et al., U.S. Pat. No. 5,124,263; International Patent Publication No. WO 95/07358, published Mar. 16, 1995, by Dougherty et al.; and Kuo et al., 1993, Blood 82:845.

Targeted gene delivery is described in International Patent Publication WO 95/28494, published October 1995.

Alternatively, the vector can be introduced in vivo by lipofection. For the past two decades, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Feigner, et. al., 1987, Proc. Natl. Acad. Sci. U.S.A. 84:7413-7417; see Mackey, et al., 1988, Proc. Natl. Acad. Sci. U.S.A. 85:8027-8031)). The use of cationic lipids may promote encapsulation of negatively charged nucleic acids, and also promote fusion with negatively charged cell membranes (Felgner and Ringold, 1989, Science 337:387-388). The use of lipofection to introduce exogenous genes into the specific organs in vivo has certain practical advantages. Molecular targeting of liposomes to specific cells represents one area of benefit. It is clear that directing transfection to particular cell types would be particularly advantageous in a tissue with cellular heterogeneity, such as pancreas, liver, kidney, and the brain. Lipids may be chemically coupled to other molecules for the purpose of targeting (see Mackey, et. al., 1988, supra). Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, biolistics (use of a gene gun), or use of a DNA vector transporter (see, e.g., Wu et al., 1992, J. Biol. Chem. 267:963-967; Wu and Wu, 1988, J. Biol. Chem. 263:14621-14624; Hartmut et at., Canadian Patent Application No. 2,012,311, filed Mar. 15, 1990).

### 8. CELL THERAPY

During development of the central nervous system (“CNS”), multipotent precursor cells (also known as neural stem cells) proliferate and give rise to transiently dividing progenitor cells that eventually differentiate into the cell types that compose the adult brain. Neural stem cells are classically defined as having the ability to self-renew (i.e., form more stem cells), to proliferate, and to differentiate into multiple different phenotypic lineages, including neurons, astrocytes and oligodendrocytes.

The non-stem cell progeny of a neural stem cell are typically referred to as:

“progenitor” cells, which are capable of giving rise to various cell types within one or more lineages. Thus, the term “neural progenitor cell” refers to an undifferentiated cell derived from a neural stem cell, and is not itself a stem cell. Some progenitor cells can produce progeny that are capable of differentiating into more than one cell type. A distinguishing feature of a progenitor cell is that, unlike a stem cell, it does not exhibit self maintenance, and typically is thought to be committed to a particular path of differentiation and will, under appropriate conditions, eventually differentiate into glia or neurons.

The term “precursor cells” refers to the progeny of neural stem cells, and thus includes both progenitor cells and daughter neural stem cells.

Neural stem cells have been isolated from several mammalian species, including mice, rats, pigs and humans. See, e.g., WO 93/01275; WO 94/09119; WO 94/10292; WO 94/16718; U.S. Pat. No. 5,968,829; and Cattaneo et al., Mol. Brain. Res., 42, pp. 161-66 (1996), all herein incorporated by reference. A population of cells exists within the adult CNS, which exhibit stem cell properties, in their ability to self-renew and to produce the differentiated mature cell phenotypes of the adult CNS. These stem cells are found throughout the CNS and particularly in the subventricular regions, and dentate gyms of the hippocampus. Growth factor-responsive stem cells can be isolated from many regions of the neuraxis and at different stages of development, of murine, rodent and human CNS tissue. These cells vary in their response to growth factors such as EGF, basic FGF (bFGF, FGF-2) and transforming growth factor alpha (TGF[alpha]), and can be maintained and expanded in culture in an undifferentiated state for long periods of time. The identification, culture, growth, and use of mammalian, including human, neural stem cell cultures, either as suspension cultures or as adherent cultures, is disclosed in Weiss et al., U.S. Pat. No. 5,750,376 and Weiss et al., U.S. Pat. No. 5,851,832, both incorporated herein by reference. Similarly, Johe, U.S. Pat. No. 5,753,506, incorporated herein by reference, refers to adherent CNS neural stem cell cultures. When cultured in suspension, CNS neural stem cell cultures typically form neurospheres.

The cells of a single neurosphere are clonal in nature because they are the progeny of a single neural stem cell. In the continued presence of a proliferation-inducing growth factor such as EGF or the like, precursor cells within the neurosphere continue to divide resulting in an increase in the size of the neurosphere and the number of undifferentiated neural cells. Neurospheres are not immunoreactive for neurofilament (NF; a marker for neurons), neuron-specific enolase (NSE; a marker for neurons) or myelin basic protein (MBP; a marker for oligodendrocytes). However, cells within the neurosphere are immunoreactive for nestin, an intermediate filament protein found in many types of undifferentiated CNS cells. (See Lehndahl et al., 60 Cell 585-595 (1990), incorporated herein by reference). Antibodies are available to identify nestin, including the rat antibody referred to as Rat401. If the neurospheres are cultured in conditions that allow differentiation, the progenitor cells differentiate to neurons and glia. The mature phenotypes associated with the differentiated cell types that may be derived from the neural stem cell progeny are predominantly negative for the nestin phenotype.

It is well recognized in the art that transplantation of tissue into the CNS offers the potential for treatment of neurodegenerative disorders and CNS damage due to injury. {See Lindvall, (1991) TINS vol. 14(8): 376-383). Transplantation of new cells into the CNS has the potential to repair damaged circuitries and to provide deficient, defect, or missing biologically active molecules, thereby restoring function. However, the absence of suitable cells for transplantation purposes has prevented the full potential of this procedure from being met. “Suitable” cells are cells that meet the following criteria: 1) can be obtained in large numbers; 2) can be proliferated in vitro to allow insertion of genetic material, if necessary; 3) capable of surviving indefinitely but stop growing after transplantation to the brain; 4) are non-immunogenic, preferably obtained from a patient's own tissue; 5) are able to form normal neural connections and respond to neural physiological signals. {See Bjorklund (1991) TINS Vol. 14(8): 319-322}. The progeny of multipotent neural stem cells obtainable from embryonic or adult CNS tissue, which are able to divide indefinitely when maintained in vitro, meet all of the desirable requirements of cells suitable for neural transplantation purposes and are a particularly suitable cell line as the cells have not been immortalized and are not of tumorigenic origin.

In one embodiment, the subject matter herein pertains to a method of treating a neurological disorder (e.g. Alzheimer's disease, Parkinson's disease, Huntington's disease, Classic Creutzfeldt-Jakob Disease, New Variant Creutzfeldt-Jakob Disease, Bovine Spongiform Encephalopathy, Gerstmann-Straussler-Scheinker Syndrome, Fatal Insomnia, Kuru, Demntia with Lewy bodies, Frontotemporal lobar degeneration, Pick's disease, Batten's Disease, Neural Ceroid Lipofuscinosis (NCL, e.g. Infantile NCL, Late Infantile NCL, Juvenile NCL, Adult NCL), Frontotemporal dementia, Semantic dementia) by administering multipotent self-renewing central nervous system neural stem cells (CNS-SC) to a subject in need thereof, wherein the multipotent self-renewing central nervous system neural stem cells produce CLN2. In a particular embodiment, the multipotent self-renewing central nervous system neural stem cells may be obtained from a human (e.g., HuCNS-SC).

Human multipotent self-renewing central nervous system neural stem cells (HuCNS-SC) have been shown to synthesize and secrete the CLN2 enzyme (see e.g. WO2006074387). CLN2 is classified as a classical soluble lysosomal hydrolases that is routed from the rough endoplasmic reticulum (RER), through the golgi apparatus, and to the lysosomes through the mannose 6-phospate receptor protein-sorting pathway. The newly synthesized hydrolases can also be secreted secondarily because a certain percentage escape recognition by the mannose 6-phosphate receptor in the golgi apparatus and end up in secretion vesicles. The extracellular enzymes specifically bind to cell surface mannose 6-phosphate receptors, and the complex is internalized and directed to the lysosomes. The acidic pH of the lysosomes causes the proteins to dissociate from the receptor, and the 6-phosphate group on mannose is, in turn, removed by lysosomal phosphatases to ensure that the internalized proteins remain and accumulate in the lysosomes and allows the receptor to recycle back to the golgi apparatus.

CLN2 is synthesized as a precursor protein (see Lin L. et al., J. Biol. Chem. 276:2249-55 (2001); Golabek et al., J Biol Chem 278:7135-45 (2003)) and, therefore, is inactive until autocatalytically cleaved and converted to the active form in the lysosomes. It has previously been demonstrated that over-expressed, secreted, recombinant CLN2 enzyme can be internalized by mammalian cells. (See Lin and Lobel, Biochem J. 357:49-55 (2001)). Receptor-dependent endocytic uptake is shown to be mediated specifically through the mannose 6-phosphate receptor present on the cell surface, and mannose 6-phosphate inhibits CLN2 internalization.

Any suitable transplantation or administration method known to those skilled in the art can be used to administer, for use as a CLN2 therapeutic, the effective amount of the multipotent self-renewing central nervous system neural stem cells and/or the medicament for treating a neurological disorder to the subject. By way of non-limiting example, transplantation may be achieved by subcortical injection, by intraventricular injection, or by any neurotransplantation protocols known to those skilled in the art. (See, e.g., U.S. Pat. No. 6,497,872, incorporated herein by reference.) A range of between about 3×10⁶ to about 1×10¹⁰ cells, for example between about 5×10⁸ to about 2×10⁹ cells or between about 1×10⁸ and about 5×10⁹ cells, can be administered to the subject. In one embodiment, a low dose of 5×10⁸ cells can be transplanted or implanted or injected or administered to the subject. In another embodiment, a high dose of 1×10⁹ cells can be transplanted or implanted or injected or administered to the subject. Those skilled in the art will recognize that the effective amount of the multipotent CNS neural stem cell population used to treat the lysosomal storage disorder can be administered either in one dose or in multiple doses.

Similarly, the medicament for treating a lysosomal storage disorder in a mammal may contain a range of between about 3×10⁶ to about 1×10¹⁰ cells, for example between about 5×10⁸ to about 2×10⁹ cells or between about 1×10⁸ to about 5×10⁹ cells. For example, in one embodiment, the medicament may contain a low dose of 5×10⁸ cells. In another embodiment, the medicament may contain a high dose of 1×10⁹ cells. Those skilled in the art will recognize that the medicament for the treatment of a lysosomal storage disorder can be administered either in one dose or in multiple doses.

In one embodiment, the multipotent CNS neural stem cell population is obtained from the subject's own neural tissue. Moreover, the multipotent CNS neural stem cell population can also be derived from neonatal, juvenile, or adult neural tissue. Those skilled in the art will recognize that the subject matter also encompasses, for use as a CLN2 therapeutic, an effective amount of a multipotent self-renewing CNS neural stem cell population in the manufacture of a medicament for treating a neurological disorder in a subject in need of such treatment. For example, the neurological disorder may one which exhibits pathology involving the accumulation of abnormal protein deposits in the brain, including, but not limited to Alzheimer's disease, Parkinson's disease, Huntington's disease, Classic Creutzfeldt-Jakob Disease, New Variant Creutzfeldt-Jakob Disease, Bovine Spongiform Encephalopathy, Gerstmann-Straussler-Scheinker Syndrome, Fatal Insomnia, Kuru, Demntia with Lewy bodies, Frontotemporal lobar degeneration, Pick's disease, Batten's Disease, Neural Ceroid Lipofuscinosis (NCL, e.g. Infantile NCL, Late Infantile NCL, Juvenile NCL, Adult NCL), Frontotemporal dementia, or Semantic dementia. Such medicaments are suitable for administration and/or transplantation into the hippocampus and/or the cortex of the subject in need of treatment for a neurological disorder. The medicament for treatment of a neurological disorder may contain between about 3×10⁶ to about 1×10¹⁰ cells, e.g., between about 5×10⁸ to 2×10⁹ cells or between about 1×10⁸ to about 5×10⁹ cells. In various embodiments, the medicament for treatment of a neurological disorder in a subject in need of such treatment contains 5×10⁸ cells (low dose) or 1×10⁹ cells (high dose). Those skilled in the art will recognize that the medicament can be administered or transplanted into the host in one dose or in multiple doses. Moreover, those skilled in the art will also recognize that the medicament is suitable for transplantation or administration by subcortical injection or by intraventricular injection. However, any other suitable transplantation or administration methods known to those skilled in the art can also be employed.

Multipotent self-renewing central nervous system neural stem cells (CNS-SC) can be administered as a CLN2 therapeutic to any animal with a neurological disorder obtained in any manner. In some instances, it may be possible to prepare CNS-SC from the recipient's own nervous system {e.g., in the case of tumor removal biopsies etc.). In such instances, the neural stem cell progeny may be generated from dissociated tissue and proliferated in vitro using any suitable method known to those of ordinary skill in the art. Upon suitable expansion of cell numbers, the CNS-SC cells may be harvested, genetically modified if necessary (e.g. so as to produce CLN2), and readied for direct injection into the recipient's CNS.

CNS-SC, when administered to the particular neural region, may form a neural graft, wherein the neuronal cells form normal neuronal or synaptic connections with neighboring neurons, and maintain contact with transplanted or existing glial cells which may form myelin sheaths around the neurons' axons, and provide a trophic influence for the neurons.

Survival of the graft in the living host can be examined using various non-invasive scans such as computerized axial tomography (CAT scan or CT scan), nuclear magnetic resonance or magnetic resonance imaging (NMR or MRI) or positron emission tomography (PET) scans. Post-mortem examination of graft survival can be done by removing the neural tissue and examining the affected region macroscopically, or may be done using microscopy. Cells can be stained with any stains visible under light or electron microscopic conditions, including stains which are specific for neurons and glia. Monoclonal antibodies may be used which distinguish and/or identify donor from host cells, specifically differences in H-2 or HLA histocompatiblity antigens. Antibodies may also be used which identify glial cell markers, inlcuding those directed to GFAP, CD11b, CNPase, MOSP, MBP, and Oligodendrocyte NS-1. Transplanted cells can also be identified by prior incorporation of tracer dyes such as rhodamine- or fluorescein-labelled microspheres, fast blue, bisbenzamide or retrovirally introduced histochemical markers such as the lac Z gene which produces beta galactosidase.

Functional integration of the graft into the host's neural tissue can be assessed by examining the effectiveness of grafts on restoring various functions, including but not limited to tests for lysosomal function.

For transplants into human subjects, those skilled in the art will recognize that any suitable method for the transplantation, administration, injection, and/or implantation of HuCNS-SC, for use as a CLN2 therapeutic, can be employed (See, e.g., U.S. Pat. No. 6,497,872, incorporated herein by reference). A range of between about 3×10⁶ to about 1×10¹⁰ HuCNS-SC cells, for example between about 5×10⁸ to about 2×10⁹ cells or about 1×10⁸ to about 5×10⁹ cells, can be administered to a human in need of treatment for a neurological disorder. Specifically, a low dose of 5×10⁸ cells or a high dose of 1×10⁹ cells can be transplanted or implanted or injected or administered to the human. Those skilled in the art will recognize that transplantation can be accomplished using any neurotransplantation protocols known to those skilled in the art. (See, e.g., U.S. Pat. No. 6,497,872, incorporated herein by reference).

For transplants into human subjects, HuCNS-SC may be administered for use as a CLN2 therapeutic in a variety of regions in the subject's CNS, including in brain tissue. HuCNS-SC may be administered in one or more of the various ventricles of the brain (e.g. left ventricle, right ventricle, thrid ventricle, fourth ventricle), and/or in the frontal and/or parietal and/or and/or occipital and/or temporal regions of the cortex. HuCNS-SC may also be administered in the left hemisphere or right hemisphere of the brain, or in both. HuCNS-SC may be implanted into the brain through a surgical procedure involving one or more injections into the subject's brain tissue and/or ventricles. The procedure is typically conducted in the operating room under general anesthesia by a neurosurgeon.

In some embodiments, three trephine holes are made over each cerebral hemisphere. The trephinations are centered over the medial aspects of the frontal and parietal lobes. Patients may receive either 5×10⁷ cells/cortical trephine and 1×10⁸ cells/ventricle trephine (for a total dose of 5×10⁸ HuCNS-SC per subject) or 1×10⁸ cells/cortical trephine and 2×10⁸ cells/ventricle trephine (for a total dose of 1×10⁹ HuCNS-SC per subject).

### 9. OTHER THERAPEUTICS

Other proteins/genes that may be employed in a therapeutic for the treatment of the neurological diseases disclosed herein, including Alzheimer's Disease, include: Aspartylglucosaminidase (AGA), Angiotensinogen (AGT), Arylsulfatase A (ARSA), Arylsulfatase B (ARSB), Acid ceramidase (ASAH), Palmitoyl-protein thioesterase 1 (CLN1), CLN5 (CLN5), Clusterin (CLU), Cellular repressor of E1a-stimulated genes (GREG), Sialic acid specific 9-O-acetylesterase (CSE-C), Cystatin C(CST3), Cystatin B (CSTB), Di-N-acetyl-chitobiase (CTBS), Cathepsin C(CTSC), Cathepsin D (CTSD), Cathepsin F (CTSF), Cathepsin H(CTSH), Cathepsin L (CTSL), Cathepsin Z (CTSZ), Deoxyribonuclease II (DNASE2), Dipeptidylpeptidase 7 (DPP7), F-box only protein 2 (FBXO2), Fc fragment of IgG binding protein (FCGBP), FLJ22662 (FLJ22662), Ferritin heavy polypeptide 1 (FTH1), Alpha-1-fucosidase (FUCA1), Alpha-glucosidase (GAA), N-acetyl-6-galactosamine sulfatase (GALNS), Gamma-glutamyl hydrolase (GGH), Alpha-galactosidase (GLA), Beta-galactosidase (GLB1), N-acetyl-glucosamine-6-sulfatase (GNS), Beta-glucuronidase (GUSB), Hexosaminidase A (HEXA), Hexosaminidase B (HEXB), Iduronate 2-sulfatase (IDS), Alpha-1-iduronidase (IDUA), Galectin-1 (LGALS1), Legumain (LGMN), Acid lipase A (LIPA), LOC196463 (LOC196463), Lysophospholipase 3 (LYPLA3), Myelin associated glycoprotein (MAG), Alpha-mannosidase (MAN2B1), Epididymis specific alpha mannosidase (MAN2B2), Beta-mannosidase (MANBA), Myeloperoxidase (MPO), Alpha-N-acetylgalactosaminidase (NAGA), Alpha-N-acetylglucosaminidase (NAGLU), Niemann-Pick disease type C2 (NPC2), Plasma glutamate carboxypeptidase (PGCP), Protective protein for beta-galactosidase (PPGB), Prolylcarboxypeptidase (PROP), Prosaposin (PSAP), Ribonuclease T2 (RNASET2), Serine carboxypeptidase 1 (SCPEP1), Sulfamidase (SGSH), S-phase kinase-associated protein 1A (SKP1A), Acid sphingomyelinase (SMPD1), and Upregulated in colorectal cancer gene 1 (UCC1). For each of the above-listed proteins, a therapeutic may include the protein, a polypeptide having at least 95%, 96%, 97%, 98%, or 99% sequence identity to the protein, a fragment thereof, a chimeric protein incorporating all of part of the protein, a nucleic acid encoding any of the proteins, mutants, fragments, and/or chimeras, vectors incorporating the nucleic acids, viruses (including gene therapy viruses) incorporating the nucleic acid and/or vector, and/or cells incorporating the nucleic acid and/or vector. Therapeutics based on any of these proteins/genes may be combined with one another or with a CLN therapeutic.

### EXAMPLES

The subject matter herein may be better understood by reference to the following Examples, which are provided by way of illustration and are in no way limiting.

### Example 1

Degradation of Cy3-labeled fAβ in microglia treated with 100 nM CLN2. Control murine microglia cells were incubated for 72 hours following uptake of Cy3-fAβ in DMEM+10% FBS+1% P/S (see FIG. 1). The experimental cells were incubated for 72 hours following uptake of Cy3-fAβ in the same medium supplemented with 100 nM CLN2. The experiments were done in triplicate in small (50 μl) coverslip dishes in a total volume of 2 ml. The average fluorescence power per cell is shown. Degradation is indicated by a decrease in fluorescence power per cell. Approximately 50 cells were examined for each condition.

### Example 2

High-throughput summary of CLN2 addback experiments. CLN2 addback experiments were performed on murine microglia with enzyme concentrations of 100 nM, 50 nM, 25 nM, and 12.5 nM (see FIG. 2). The percent of Cy3-fAβ degraded in the microglia for each condition was compared. The experimental protocol was similar to FIG. 1, but 384-well high-throughput plates were used in order to conserve the amount of enzyme consumed per experiment. At least six wells per enzyme addback condition, with approximately 5000 microglia per well in a total volume of 50 μl media, were used. Controls with 10 mM mannose-6-phosphate were included for each CLN2 addback concentration. The percent degradation was calculated from each corresponding mannose-6-phosphate control set. It was found that there was −30% degradation of the Cy3-fAβ when 100 nM of CLN2 was added to microglia (as compared to a mannose-6-phosphate control). This decreased to 22% degradation at 50 nM CLN2, 19% at 25 nM, and insignificant (2%) degradation at 12.5 nM.

### Example 3

Protein Therapy for Treatment of Alzheimer's Disease

Production of Recombinant CLN2 Protein in CHO Cells. Full-length human CLN2 cDNA was cloned into pMSXND1 vector (S-J Lee and D Nathans, 1988 J. Biol. Chem. 263 3521-7). The resulting plasmid contains a CLN2 expression cassette driven by the metallothione I promoter, a neomycin-resistance cassette for G418 selection, and a dihydrofolate reductase expression cassette for methotrexate (MTX) resistance. CHO cells were transfected with linearized plasmid using lipofectamine (Gibco) and stably transfected clones were isolated after G418 selection. The TPP1 activity in conditioned medium was assayed and the highest expressor was treated with NITX to select for clones that had undergone gene amplification. The TPP1 activity level in conditioned medium was increased by 15 fold over endogenous level after transfection and G418 selection and further increased to >1000-fold over untransfected cells after MTX amplification. The TPP1 activity assay was conducted using a modification of the method of Vines and Warburton (Biochem. Biphys. Acta. 1998 1384 pp 233-42). Unless indicated otherwise, samples were preincubated in 150 mM NaCl/0.1% triton X-100/50 mM formate pH 3.5 for 30 minutes at 37 C to convert proCLN2 to active enzyme.

Culture conditions were evaluated in an attempt to optimize the production system. Various growth media were compared including different types of standard media with or without fetal bovine serum, reduced serum medium, and specialized low-protein formulations for CHO cell. Gibco CHO-S-SFM II media resulted in the best yield. However, this media contains protein components that could interfere with downstream purification. Thus, even though the protein-free medium DMEM/F12 gave approximately a two-fold lower yield, this medium was chosen for production purposes.

Purification and characterization of recombinant CLN2 protein. CHO cell culture medium was concentrated by ultrafiltration through YM-30 membrane and buffer exchanged into low salt solution. The material was subjected to anion-exchange chromatography on a Uno Q12 column (BioRad). The TPP1 activity profile revealed that the CLN2 protein represents the major OD280 peak that elutes at about 150 mM NaCl. Comparison of the proteins present in the source material and the peak anion exchange fractions by denaturing PAGE (10% NuPAGE, Novex) and Coomassie blue staining indicates that the 65 KDa CLN2 protein is the major protein in the conditioned media and that most minor components are removed by ion exchange chromatography.

Peak fractions were pooled and applied to a Superose-12 gel filtration column. The protein elutes as a single peak that, in comparison to gel filtration standards, elutes as a globular protein of 65 kDa. This indicates that the CLN2 protein precursor exists as a monomer in solution at pH 7.4. Note that all chromatography was performed at slightly alkaline pH (pH 7.4 to 8.0) as the inactive CLN2 precursor undergoes autoactivation to active 46 KDa CLN2 protein at acidic pH (see below).

Ideally, the recombinant CLN2 protein used for enzyme replacement therapy should contain mannose 6-phosphate to allow its endocytosis and delivery to the lysosome. To investigate its mannose 6-phosphorylation state, recombinant proCLN2 was applied to a column of immobilized soluble cation-independent mannose 6-phosphate receptor. The column was washed with column buffer, column buffer containing glucose 6-phosphate (which does not bind to the mannose 6-phosphate receptor and potentially could release some non-specifically bound material), mannose 6-phosphate, and glycine buffer (to elute tightly or non-specifically bound material). The fractions were analyzed by the TPP1 activity assay after autoactivation or by SDS-PAGE (data not shown). Essentially all of the CLN2 protein was retained on the column and was specifically eluted with mannose 6-phosphate. This demonstrates that recombinant CLN2 protein produced in CHO cells is mannose 6-phosphorylated.

Autocatalytic processing of CLN2 protein/TPP1. When maintained at neutral or alkaline pH, purified CHO-cell produced human recombinant CLN2 protein has an apparent size of about 65 KDa by denaturing PAGE. Edman degradation revealed that the amino terminus (SYSPE . . . ) corresponds to residue 20 of the translated CLN2 message. This indicates that CLN2 protein is synthesized as a preproprotein whose signal sequence is cleaved after residue 19 to generate proCLN2 protein. Upon incubation at acidic pH the 65 KDa protein is converted to a 46 KDa species whose amino terminus (LHLGV) corresponds to residue 196 of the translated CLN2 message. This amino terminus is identical to endogenous CLN2 protein isolated from human brain (Sleat et al 1997, Science 277 pp 1802-1805). Kinetic analysis of highly purified recombinant CLN2 protein produced in insect cells indicate that the proteolytic processing is accompanied by acquisition of enzymatic activity. (Note that the CHO cell preparation is used for all other experiments described in this application. The insect cell preparation was used for this analysis before the CHO cell preparation was available. Preliminary experiments indicate that with respect to autocatalytic processing, the two preparations are essentially identical). These data indicate that the CLN2 protein is synthesized as an inactive proenzyme and upon acidification undergoes autolysis to an enzymatically active species.

Uptake of CLN2 protein by cultured neurons. To determine the ability of the recombinant enzyme to be delivered to neurons, rat cerebellar granule neurons were cultured with increasing concentrations of CLN2 protein for one day and analyzed intracellular TPP1 activity using the kinetic assay.

Cerebellar granule neurons were prepared from postnatal day 8 Sprague-Dawley rat pups as described (Meiners, et al., (1999) J Neurosci 19, 8443-8453). The cells were plated into 48-well plates at a density of 150,000 cells/cm². When cells were confluent, media were replaced with fresh media containing the indicated concentrations of purified recombinant human CLN2 protein (0.5 ml). Immediately before processing, cells were washed 3 times with PBS (0.5 ml) at room temperature and then rapidly cooled by placing dishes in an ice water bath. The cells were lysed by adding 1% Nonidet P40/10 mM Tris pH 7.5/150 mM NaCl (0.2 ml/well) and incubated for 1 hour at 4° C. on a rocking platform. The lysate was transferred to microfuge tubes and centrifuged for 20 min at 13,000×g. The supernatant was used for enzyme activity and protein (Lowry, et al. (1951) J Biol Chem 193, 265-275) assays.

Depending on how the samples were processed, the TPP1 activity reflects either the mature CLN2 protein or both precursor and mature CLN2 protein present within the neurons. At concentrations of recombinant CLN2 protein where there was a significant increase of TPP1 activity over endogenous levels (>3 nM in the absence of M6P and >10 nM in the presence of M6P), thus allowing reliable estimation of the endocytosed protein, ˜80% of the endocytosed CLN2 protein was in the mature form. This indicates that the enzyme is targeted to an acidic intracellular compartment. Also, the uptake did not saturate at high CLN2 protein concentrations. However, the M6P inhibitable uptake was saturable (EC50 6-8 nM), indicating that at high concentrations, there was considerable uptake through MPR-independent mechanisms. Nonetheless even when MPR-independent mechanisms predominated, ˜80% of the endocytosed enzyme was converted to the active form, demonstrating proper lysosomal targeting of the recombinant CLN2 protein.

The above example provides a production system for recombinant human CLN2 protein and demonstrates that this protein can be delivered to lysosomes of cultured neurons. Similar CHO-based production systems have been used to produce large quantities of other lysosomal enzymes for protein characterization and enzyme replacement studies (Kakkis, et al. (1994) Protein Expr Purif 5, 225-232; Ioannou et al. (1992) J Cell Biol 119, 1137-1150; Bielicki, J., et al. (1998) Biochem J 329, 145-150; Martiniuk et al. (2000) Biochem Biophys Res Commun 276, 917-923). Consistent with the findings herein, overexpression of a given recombinant lysosomal enzyme typically results in its disproportionate secretion (Kakkis, Ioannou).

The properties of the CLN2 protein precursor differ in a number of aspects from that of the mature protein. First, the precursor is enzymatically inactive but, upon acidification, is autocatalytically processed to the mature, active form. Second, the mature enzyme is rapidly inactivated when incubated at 37° C. at neutral pH (Sohar et al. (1999) J Neurochem 73, 700-711; Vines. D. and Warburton. M. J. (1998) Biochem Biophys Acta 1384, 233-242.

In contrast, the proenzyme is stable at neutral pH and can subsequently be converted to the active form. Finally, the quaternary structure and physical properties of the proenzyme and mature enzyme appear to be quite different. For instance, published procedures for purification of mature CLN2 protein/TPP1 from mammalian tissues utilize detergent to maintain the protein in solution (Vines and Warburton; Doebber et al. (1978) Endocrinology 103, 1794-1804; McDonald et al. (1985) Biochem Biophys Res Commun 126, 63-71; Watanabe et al. (1992) Biochem Int 27, 869-877; Page et al. (1993) Arch Biochem Biophys 306, 354-359; Junaid et al. (2000) J Neurochem 74, 287-294), and gel filtration analysis indicates that the mature protein forms aggregates of 250 to 700 KDa (McDonald et al.; Page et al.). In contrast, the proenzyme behaves as a soluble monomer as shown herein.

Recombinant CLN2 protein produced from CHO cells has a number of properties that appear useful for enzyme replacement therapy in Alzheimer's disease. First, as the proenzyme is inactive and stable in an extracellular environment until delivery to the lysosome, and the mature form is unstable with little activity at neutral pH, concerns about unwanted proteolysis of extracellular structures by TPP1 activity after enzyme administration should be minimized. Second, the protein is efficiently delivered to the lysosome by M6P mediated endocytosis and possibly by other endocytic mechanisms. Third, the internalized active protein has a long half-life within lysosome, which will be important in considering dosing regimes.

The results herein show that large quantities of recombinant CLN2 protein can be readily obtained from the CHO cell system. This will facilitate enzyme replacement studies in animal models and development of novel delivery methods for treatment of Alzheimer's disease.

All base sizes and amino acid sizes, and all molecular weight or molecular mass values given for nucleic acids or polypeptides are approximate, and are provided for description.

Such methods are described in further detail in, e.g., U.S. patent application Ser. No. 10/255,317 to Lobel, which is hereby incorporated herein by this reference.

### Example 4

Cell Therapy for Treatment of Alzheimer's Disease

Transplantation of HuCNS-SC. Human CNS stem cells (HuCNS-SC) are a cell therapy product comprised of an injectable suspension of human neural stem/progenitor cells. HuCNS-SC are transplanted in Alzheimer's subjects in part to determine if the transplanted cells secrete the TPP1 enzyme into the brains of affected individuals. HuCNS-SC have been shown to produce both TPP1 and a related enzyme, palmitoyl-protein thioesterase 1 (PPT1), thereby providing a scientific justification for enzyme replacement and cellular rescue in this indication. In preclinical models of PPT1 deficiency, the corresponding enzyme activity increases with time after transplantation. Thus, the safety of HuCNS-SC in the treatment of neurological disorders, including Alzheimer's disease, can be investigated.

A range of between about 3×10⁶ to about 1×10¹⁰ HuCNS-SC cells, for example between about 5×10⁸ to about 2×10⁹ cells or about 1×10⁸ to about 5×10⁹ cells, can be administered to a mammal suffering from a lysosomal storage disorder. For example, HuCNS-SC are surgically administered by subcortical and intraventricular injection. Two doses of cells are administered: a low dose of 5×10⁸ cells injected at a concentration of 5×10⁷ cells/ml and a high dose of 1×10⁹ cells injected at a concentration of 1×10⁸ cells/ml.

Preoperative MRI is used to select subcortical target sites in the anterior frontal, anterolateral frontal, and parietal lobes where the cortical mantle (brain surface to ventricular surface) is at least 20-30 mm thick. Target sites are selected so as to avoid eloquent cortex and other critical brain structures. Cortical thickness is measured directly off the MRI scan images. Four burr holes are placed on each side of the skull, three for access to the selected subcortical sites and one for access to the lateral ventricle. A stereotactic navigation instrument such as the StealthStation® (Medtronic, 510KNo. KOO1 153) may be used in addition to anatomic landmarks to aid in the anatomic localization of the burr holes corresponding to the targeted subcortical injection sites. The stereotactic navigation instrument will only be used for planning purposes and as an aide in locating anatomic structures; it will not be used for injection of HuCNS-SC. HuCNS-SC are injected subcortically to a depth of approximately 20 mm below the cortical surface. One ml of HuCNS-SC suspension is injected manually over 3-5 minutes. The rate of injection is hand-modulated based on the ability of the brain to absorb the volume without visible reflux back along the needle track. The needle is left in place for 2-3 minutes after each injection and then withdrawn slowly. For the ventricular injections, the frontal horn of the lateral ventricle is cannulated.

The selected catheter should be a well established neurosurgical instrument that is used for atraumatic access to the ventricle and for injection of antibiotics, chemotherapeutics or dye into the ventricle. Approximately 5 ml of the subject's cerebrospinal fluid (CSF) are withdrawn through the catheter and set aside to be used to flush the catheter after injection. Two ml of HuCNS-SC suspension is injected manually over 2-3 minutes. The catheter is flushed with 2 ml of the subject's CSF over 2-3 minutes and then slowly removed.

At the conclusion of the procedure, each burr hole is closed by placing Surgifoam absorbable gelatin sponge (Ethicon, PMA No. P990004) in the burr hole above the dura, and the galea closed with Vicryl sutures (Ethicon) and the skin closed with Monocry sutures (Ethicon). Subjects are monitored in the intensive care unit at least overnight after surgery.

Immunosuppression. HuCNS-SC Cell Therapy is an allogeneic transplant. The cells are implanted into subjects without donor and recipient tissue-type matching. Thus, immunosuppression may be necessary to prevent rejection of the transplanted HuCNS-SCs.

For example, combination immunosuppression therapy using corticosteroids (10 mg/kg/day) and Prograf® (0.3 mg/kg/day) may be employed for up to 1 year post-transplant. Specifically, Prograf® can be initiated prior to the transplant and maintained up to one year post-transplant (dosage is reduced to 0.1 mg/kg/day 30 days following transplant). Prograf® administration can be monitored for adverse experiences at specific intervals post-transplant to assess tolerability. Toxicokinetic assessment of Prograf® blood levels will permit customized dosing for each subject. In addition, corticosteroids can be administered immediately prior to surgery for up to 5 days postoperatively and then tapered to discontinuation.

Such methods are described in further detail in, e.g., International Application No. WO2006US00490 to Nobuko et al., which is hereby incorporated herein by this reference.

### Example 5

Human Gene Therapy for Alzheimer's Disease Using the AAV2_{CU}hCLN2 Vector.

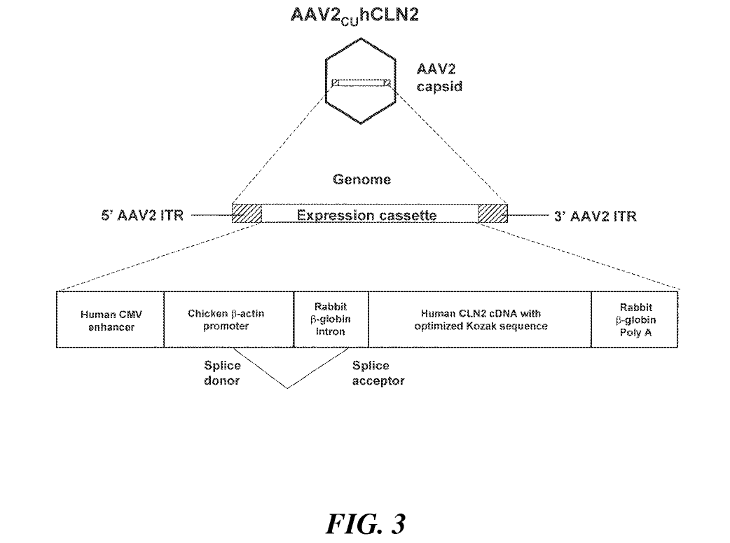

The AAV2CUhCLN2 vector (see FIG. 3. from Crystal et al., Hum. Gene Ther. 15, 1131-1154, 2004) is based on the serotype 2 adeno-associated virus. AAV is a small nonenveloped icosahedral parvovirus with a 4.7-kb single-stranded DNA genome. AAV2 is a naturally replication-defective virus that depends on adenovirus (Ad) or herpes simplex virus gene products for replication (Berns and Giraud, 1996). The absence of any detectable pathology from wild-type AAV2 infections coupled with its ability to remain latent promoted its development as a gene transfer vector (Backlow, 1988; Muzyczka, 1992; Carter, 2000). Recombinant vectors based on AAV2 are effective in long-term gene transfer to skeletal and cardiac muscle, liver, brain, and retina (Kessler et al., 1996; McCown et al., 1996; Fisher et al., 1997; Flannery et al., 1997; Koeberl et al., 1997; Maeda et al., 1998; Bennett et al., 1999; Chen et al., 2000; Paterna et al., 2000). AAV2 vectors are designed in a fashion such that all viral genes are replaced by an expression cassette for the transgene, leaving intact the essential cis elements of the genome, the inverted terminal repeats (ITRs), DNA packaging signal, and the replication origin (Backlow, 1988). Replication and packaging of AAV2 vectors requires all AAV2 and Ad helper functions to be provided in trans.

The strategy proposed herein is for the intracranial delivery of an AAV2 vector encoding the human CLN2 cDNA. The advantages of this direct injection strategy to treat Alzheimer's disease include bypassing the blood-brain barrier and long-term expression of the transgene product (TPP1) in the organ (brain) that is most affected.

Each individual will receive a total dose of 3.6×10¹² particle units of AAV2_{CU}hCLN2, divided among 12 locations delivered through 6 burr holes (2 depths through each hole), 3 burr holes per hemisphere. The exact locations of the administration of the vector will be decided on a case-by case basis as described above. Preoperative MRI will be used to guide the sites for the regions of administration and hence the burr holes. For standardization, the coronal sutures and bony landmarks of the cranium will be used to draw a line in the sagittal plane on the MRI. There will be 12 target sites per brain delivered through 6 burr holes (2 locations at varying depths through each hole) with 3 in each hemisphere.

Neurosurgical administration of AAV2_{CU}hCLN2 will involve drilling six small burr holes in the calvarium under general anesthesia in order to gain access to defined subcortical regions of the brain with a fine catheter. Individuals will be prepared for anesthesia and surgery in the standard fashion. They will fast from food and liquids after midnight, prior to the surgical procedure. Individuals will be transported to the operating room where monitors (three-lead modified EKG, pulse oximeter, capnography [a monitor that displays the level of exhaled carbon dioxide in the breathing tube]) will be applied; routine premedications will be administered as needed. Subjects will require a radial arterial line for continuous blood pressure monitoring and blood sampling.

The individual's head will then be prepped and draped, as anatomically indicated, in a standard sterile fashion using a betadine/alcohol solution. At the completion of successful burr holes (see above), catheters will be put in place. Intravenous mannitol (typically 1.0 g/kg, but at the anesthesiologist's discretion) may be given as needed to reduce brain edema throughout the period of vector administration.

A total of 3.6×10¹² particle units of the AAV2_{CU}hCLN2 vector will be administered to the whole brain. The vector will be administered to each of the six sites in parallel. Each of the six sites will receive 0.6×10¹² particle units, half at one site at the bottom of the needle track and half at a site approximately half-way up the needle track. The vector will be delivered by a microperfusion pump that accommodates six syringes simultaneously. The vector will have a concentration of approximately 2.0×10⁹ particle units/μl, and will be delivered at 2.0 μl/min at each site for a total of 300 μl/site (6.0×10¹¹ particle units/site). After the specified dose is administered to the six sites, the catheters will remain in place for 5 min to assure tissue penetration. The catheters will be withdrawn to approximate half-way from the bottom of the needle track to the brain surface, and the remaining 50% of the dose will be administered, in parallel, to each of the six sites exactly as described above. After the period of vector administration (estimated to be 2.5 to 3.0 hr), the surgical wounds will then be closed by standard techniques. A postoperative MRI will be performed within the first 48 hr after the surgical procedure. The exact time the MRI will be performed will be determined at the discretion of the neurological team. Each patient will be monitored postoperatively in a recovery room or intensive care unit until he/she is stable to be transferred to the Children's Clinical Research Center unit individuals will be discharged horn the hospital at the discretion of the attending neurosurgeon. The total length of hospitalization is estimated at less than 7 days, assuming no postoperative complications occur (Janson et al., 2002).

Such methods are described in further detail in, e.g., Crystal R. G. et al. Human Gene Therapy. (2004) 15:1131-1154.

### Example 6

Primate Gene Therapy for Alzheimer's Disease Using the AAV2_{CU}hCLN2 Vector.

African green monkeys (Cercopithecus aethiops sabaeus, male, 4.0 to 6.5 kg, feral, estimated age of 5 to 10 years) may be used as subjects for these experiments. Each nonhuman primate will receive intracranial administration of a total dose of 3.6×10¹⁰ PU (low dose) or 3.6×10¹¹ PU (high dose) of the AAV2_{CU}hCLN2 vector or 3.6×10¹¹ PU of the AAV2_{CU}Null control vector or PBS at 1 μl/min for a total volume of 180 μl (15 μl at each of 12 locations). An additional control may be Sham-injected animals, which involves insertion of the injection needle into all locations but no administration. Six burr holes (three symmetrical holes per hemisphere) are drilled through the skull of anesthetized monkeys, followed by the lowering of the syringe sequentially to two defined depths per burr hole for injection of vector or vehicle. Injections are made to the head of the caudate nucleus and overlying the cerebral cortex (16.2 mm apart), the body of the caudate nucleus and the overlying cerebral cortex (19.2 mm apart) and the hippocampal formation and overlying cerebral cortex (31.2 mm apart).

Monkeys w it be anesthetized with pentobarbital (25 mg/kg, intravenous) and the heads shaved. Preoperatively, each monkey will receive (intramuscularly) atropine (0.5 mg), long-acting penicillin (600,000 units), and iron dextran (150 mg). An intravenous line should be inserted with 0.9% saline infusion. An endotracheal tube will be inserted and monkeys will be monitored continually and receive assisted ventilation if required. The animal is first set up in the stereotaxic head holder and midline incisions are made and the muscles retracted to each side. Precise holes are drilled at the six stereotaxic points of entry, using a Foreman high-speed drill with a diamond ball bit. A stereotaxic carrier containing a Stoelting microsyringe injector with a Hamilton 710 series 100-μl syringe and a replaceable 2-in., 22-gauge beveled needle is attached to the stereotaxic frame. Ninety microliters of the appropriate vector or PBS is then drawn up into the chamber. Cortical injections will be made first and then the cannula is lowered to the site of the caudate or hippocampus. After each injection is made at a rate of 1 μl/min, 2 min should be allowed to elapse before the cannula is withdrawn slowly and repositioned for the next injection. The syringe is refilled once with 90 μl to inject all 12 sites sequentially. After the final injections, the wound is closed by approximating the muscle layers with chromic suture, closure of the subcutaneous tissue also with chromic suture, and a skin closure using Vicryl. The wound is then sealed with collodion and the monkey removed from the stereotaxic holder and observed in the recovery area until it is awake, at which time it will be returned to its cage. Monkeys may, be assessed three times a day during recovery from surgery and daily thereafter.

Such methods are described in further detail in, e.g., Hackett N. R. et al. Human a Gene Therapy. (2005) 16:1484-1503.

### Example 7

Mammalian Gene Therapy for Alzheimer's Disease Using the AAV2_{CU}hCLN2 Vector.

AAV vector production. The genomic structures of AAV2_{CU}hCLN2 and AAV5_{CU}hCLN2 are identical to each other and contain serotype-2 inverted terminal repeats and the human CLN2 cDNA under the control of the cytomegalovirus enhancer chicken β-actin promoter. The AAV2_{CU}NULL control vector is also similar except that the human CLN2 cDNA is substituted with a noncoding DNA sequence of equal size. The detailed description of the recombinant genomes used in this study and the production and characterization of AAV2_{CU}hCLN2 and AAV2_{CU}NULL have been reported previously by Sondhi et al. (Gene Ther. 12, 1618-1632, 2005). AAV5_{CU}hCLN2 is produced by cotransfection of helper plasmid pPAK-MA5 and CLN2 plasmid pAAV2-CAG-hCLN2 in a 10-Stack CellFactory (VWR Scientific, West Chester, Pa.) containing human 293 cells. Seventy-two hours after transfection, cells are harvested, and viral lysate is collected, treated with benzonase, clarified by centrifugation, and purified by discontinuous iodixanol gradients. Pooled fractions containing AAV5 are treated with 0.5% octyl glucopyranoside, loaded onto a Q-HP anion exchange column, eluted using a linear 0-1 M NaCl gradient, and concentrated by dialysis against 120 g/L dextran-40. In vitro enzyme assays can be used to verify that AAV2_{CU}hCLN2 and AAV5_{CU}hCLN2 express TPP1. Viral vectors should be made under good manufacturing practice, and the titers of AAV2_{CU}hCLN², AAV2_{CU}NULL, and AAV5_{CU}hCLN2 should be approximately 2.0×10¹¹ genome copies (gc)/ml.

Injection of AAV vectors into the mouse brain. All procedures described herein will be performed under a protocol approved by the Institutional Animal Care and Use Committee. Mice will be housed under 12 h light/dark cycle and provided with food and water ad libitum. In one protocol, mice at 6 or 10 weeks of age will undergo stereotaxic brain surgery and will be injected into two sites along a single needle tract with AAV2_{CU}hCLN2, AAV2_{CU}NULL, or saline. Injections will be performed in the thalamus (2.00 mm caudal to bregma, 1.75 mm right of midline, 3.50 mm ventral to pial surface) and hippocampus (2.00 mm caudal to bregma, 1.75 mm right of midline, 1.75 mm ventral to pial surface) of the right hemisphere. Three microliters (6.0×10⁸ gc) of either vector will be dispensed with a Hamilton syringe (Hamilton, Reno, Nev.) into each structure for a total of 6 μl (1.2×10⁹ gc) per brain. In an alternative protocol, mice at 6 weeks of age will undergo stereotaxic brain surgery and receive six injections (three per hemisphere) in six different needle tracts with AAV2_{CU}hCLN2 (n=8) or AAV5_{CU}hCLN2 (n=9). Injections will be done bilaterally in the motor cortex (1.00 mm rostral to bregma, 1.25 mm from midline, 1.25 mm ventral to pial surface), thalamus (2.00 mm caudal to bregma, 1.75 mm from midline, 3.50 mm ventral to pial surface), and cerebellum (6.00 mm caudal to bregma, 1.50 mm from midline, 1.50 mm ventral to pial surface). Three microliters (6.0×10⁸ gc) of either vector will be injected with a Hamilton syringe for a total of 9 μl (1.8×10⁹ gc) per hemisphere and 18 μl (3.6×10⁹ gc) per brain. The injections in both protocols will be performed at a rate of 0.5 μl/min, and the needle should be left in place for 2 min after each injection to minimize upward flow of viral solution after raising the needle.

Such methods are described in further detail in, e.g., Passini M. A. et al. The Journal of Neuroscience. (2006) 26:1334-1342.

### Example 8

Administration of CLN2 Protein to Mammals

Transgenic mice that develop amyloid-beta plaques have been used as models for Alzheimer's disease for nearly a decade. These animals have been the primary means by which new therapeutic approaches for Alzheimer's disease have been tested.

One defense mechanism against the deposition of amyloid-beta plaques in the brain involves a particular cell-type known as microglia. Described herein is a method to apply CLN2 enzyme to the brains of transgenic mice which have developed amyloid-beta plaques. Mouse brains are monitored following treatment to assess the effectiveness of the CLN2 therapeutic.

Manual restraint or mechanical restrain of animals. The animals are placed in a standard stereotaxic frame after initial anesthesia. Surgical and non-surgical procedures varying in duration from 1 to 2.5 hours are performed, and the animal is removed from the stereotax prior to waking-up.

Preoperative procedures. The animals are anesthetized with an intraperitoneal injection of avertin (200 mg/kg). Supplemental doses of avertin (40 mg/kg) are given as needed determined by response to foot pinch. Approximate time under anesthesia is 1-2.5 hrs.

Craniotomy and cranial window preparation for multiphoton microscopy. After anesthesia as described above, the scalp is shaved clean of fur and swabbed with povinde/iodine solution. A longitudinal incision is made and a circular section of scalp is cut from directly behind the eyes to between the ears (approx. 10 mm diameter). The mouse is placed in a stereotaxic apparatus, and the connective tissue and aponeurosis is then cleaned with swab. A cranial windowing procedure modified from Yuan et al (Cancer Research (1994). 54, 4564-68) is performed as described herein. A 5-8 mm circle is drawn over the frontal and parietal regions of the skull bilaterally. Using a high speed microdrill with a 0.9 mm burr tip, a groove around the drawn circle is made. The groove is continuosuly and evenly made thinner until the skull becomes flexible and can be picked away around the entire circumference, except at the most anterior point of the circle, where a major vessel lies directly beneath the skull. Cold, sterile PBS is applied during the process to avoid thermal injury to the cortex. The tip of a blunt pair of forceps is inserted under the posterior edge of the circle and the bone is gently lifted to separate from the dura mater. The dura is kept moist with sterile saline and gel foam. A nick is made close to the sagittal sinus, and following insertion of microscissors, the dura is carefully cut away from the surface of the cortex in both hemispheres. The surface is kept moist with saline and gel foam. The site is filled with saline and a glass coverslip of roughly the same diameter is gently dropped onto the site, such that the space beneath and to the edges is filled with saline. Using histocompatible cranioplastic cement/glue, a seal is made around the coverslip and the animal is placed on a heating pad until the glue is dry. The mouse is transferred to the stage of a multiphoton imaging scope, which is modified to contain mouse restraints (ear bars and bite bar) analogous to the stereotaxic frame. Using a pulsed Ti:Sapphire laser tuned to 750 nm, the cortex of the mouse is imaged. The animal is removed from the microscope and stereotax and placed on a heating pad to recover, while the temperature is monitored as the mouse wakes up.

Multiphoton imaging. After anesthesia and administration of the fluorescent contrast reagent, the mouse is placed in a stereotaxic apparatus. The fluorescent contrast reagents used include a fluorescent dextran for angiogram labeling (no known toxicity) and a novel small molecule (methoxy-XO4 or PIB series of dyes, with no known toxicity), which can be administered via intraperitoneal or intravenous injection using a 27 gauge needle at a dose of 2-10 mg/kg. These compounds are injected from a stock concentration of 50 mg/ml. The microscope objective is positioned over cranial window, and fluorescent images are recored for 20-60 min. Anesthesia is monitored throughout the imaging session by toe-pinch and top-up doses are administered to maintain anesthesia.

Chronic multiphoton imaging. After anesthesia and administration of the fluorescent contrast reagent, the mouse is placed in a stereotaxic apparatus. The fluorescent contrast reagents used include a fluorescent dextran for angiogram labeling (no known toxicity) and a novel small molecule (methoxy-XO4 or PIB series of dyes, with no known toxicity), which can be administered via intraperitoneal or intravenous injection using a 27 gauge needle at a dose of 2-10 mg/kg. These compounds are injected from a stock concentration of 50 mg/ml. The microscope objective is positioned over cranial window, and fluorescent images are recored for 20-60 min. The animal is allowed to recover, and the procedure is repeated every 7 days for a period of 2 weeks following the initial imaging session. After the final imaging session, the animal is euthanized with 400 mg/kg IP Avertin and the brain removed.

Purified Recombinant Enzyme application. After anesthesia and administration of the fluorescent contrast reagent, the mouse is placed in a stereotaxic apparatus. Recombinant TPP1 enzyme (10 nM) is added topically to the surface of the exposed brain before the coverslip is cemented in place.

Endpoints of study. In the studies described herein, animals are to be sacrificed 48 hours after administration of the recombinant protein. The right hemisphere is treated with a sham injection (for example, vehicle only) whereas the contralateral hemisphere will contain the active enzyme (TPP1). The number of plaques are assessed before and after treatment by in vivo imaging with multiphoton microscopy, and by subsequent histological examination. Studies to date show that there is about 15% difference between the right and left hemispheres of any individual animal.

A pilot experiment according to Example 8 was carried out using as subjects 1 Tg2576 mouse and 2 B6C3 mice. TPP1 pro-enzyme was topically applied at the surface of the cranial window. After a 30-minute incubation, the cranial window was sealed and plaques were imaged and recorded at several locations. At the next imaging sessions, 2 and 5 days later, there were no noticeable changes in number or size in the plaques observed. The absence of noticeable change may have resulted from a number of sources unrelated to basic efficacy of TPP1 in treating Alzheimer's disease, including treating an insufficient number of subjects to reveal a significant difference, administering an insufficient dose, allowing insufficient time for incubation or delay before comparative imaging, among others.

### Example 9

Administration of CLN2 Gene Therapeutic to Mammals

This method is similar to the method described in Example 8, but instead of “Purified Recombinant Enzyme application,” the following is performed:

Craniotomy and neocortical injection of AAV virus. After anesthesia as described above, the scalp is shaved clean of fur and swabbed with povidine/iodine solution. A longitudinal incision is made from directly behind eyes to between ears (approx. 10 mm long). The mouse is placed in a stereotaxic apparatus, and the connective tissue and aponeurosis is then cleaned with swab. Using a high-speed microdrill with a 0.9 mm burr tip, a small craniotomy is made, less than 1 mm in diameter. Using stereotaxic coordinates, 4 μl of virus {adeno-associated virus containing the GFP gene (green fluorescent protein) or the CLN2 gene (pepstatin-insensitive carboxyl protease I) 1 mg/ml} is injected with a sterile 27 gauge Hamilton syringe into the neocortex. The scalp is closed and sutured.

Endpoint: similar to that described in Example 8, but sacrifice is made 3 weeks after administration of the AAV (to permit time for expression of vector), and the sham injection includes, for example, AAV encoding the non-toxic protein, green fluorescent protein.

1. A method of treating Alzheimer's Disease comprising administering to a subject in need of such treatment a CLN2 therapeutic that comprises a nucleic acid encoding a protein having at least 95% sequence identity to a sequence selected from the group consisting of SEQ ID NO:1 through SEQ ID NO:34, wherein the protein has a beta-amyloid degradation activity.

2. The method of claim 1, wherein the nucleic acid encodes a protein having at least 95% sequence identity to a sequence selected from the group consisting of SEQ ID NO:5 through SEQ ID NO:11.

3. The method of claim 1, wherein the nucleic acid encodes a protein having at least 95% sequence identity to SEQ ID NO:5.

4. The method of claim 1, wherein the nucleic acid encodes a protein having at least 99% sequence identity to SEQ ID NO:5.

5. The method of claim 1, wherein the nucleic acid encodes a protein having 100% sequence identity to a sequence selected from the group consisting of SEQ ID NO:1 through SEQ ID NO:34.

6. The method of claim 1, wherein the nucleic acid encodes a protein having 100% sequence identity to SEQ ID NO:5.

7. The method of claim 1, further comprising administering to the subject a second CLN2 therapeutic that comprises a polypeptide having at least 95% sequence identity to a sequence selected from the group consisting of SEQ ID NO:1 through SEQ ID NO:34.

8. The method of claim 7, wherein the polypeptide has at least 95% sequence identity to a sequence selected from the group consisting of SEQ ID NO:5 through SEQ ID NO:11.

9. The method of claim 7, wherein the polypeptide has at least 95% sequence identity to SEQ ID NO:5.

10. The method of claim 7, wherein the polypeptide has at least 99% sequence identity to SEQ ID NO:5.

11. The method of claim 7, wherein the polypeptide has 100% sequence identity to SEQ ID NO:5.

12. The method of claim 7, wherein the polypeptide has 100% sequence identity to a sequence selected from the group consisting of SEQ ID NO:1 through SEQ ID NO:34.

13. The method of claim 1, wherein the nucleic acid of the therapeutic further encodes a transcytosis peptide.

14. The method of claim 1, wherein the nucleic acid of the therapeutic hybridizes to a nucleic acid comprising a sequence selected from the group consisting of SEQ ID NO:35 through SEQ ID NO:58 under the following hybridization conditions: 50% formamide, and 5× or 6×SCC.

15. The method of claim 1, wherein the therapeutic comprises a vector comprising the nucleic acid.

16. The method of claim 15, wherein the vector is an expression vector.

17. The method of claim 16, wherein the vector further comprises a promoter.

18. The method of claim 17, wherein the promoter is a constitutive promoter.

19. The method of claim 17, wherein the promoter is an inducible promoter.

20. The method of claim 15, wherein a virus comprises the vector.

21-26. (canceled)
